(19) United States
(12) Patent Application Publication
(10) Pub. No.: US 2024/0379236 A1
(43) Pub. Date: Nov. 14, 2024

(54) METHOD FOR THE DIAGNOSIS AND/OR CLASSIFICATION OF A DISEASE IN A SUBJECT

(71) Applicants: CHRISTIAN-ALBRECHTS-UNIVERSITÄT ZU KIEL, Kiel (DE); FACHHOCHSCHULE KIEL, Kial (DE); FREIE UNIVERSITÄT BERLIN, Berlin (DE); MAX-PLANCK-GESELLSCHAFT ZUR FÖRDERUNG DER WISSENSCHAFTEN E.V., Munchen (DE)

(72) Inventors: Franz-Josef MÜLLER, Felde (DE); Björn Fabian BRÄNDL, Kropp (DE); Bernhard SCHULDT, Dusseldorf (DE); Christian ROHRANDT, Nubbel (DE); Alena VAN BÖMMEL, Leipzig (DE); Helene KRETZMER, Berlin (DE)

(21) Appl. No.: 18/688,071

(22) PCT Filed: Sep. 6, 2022
(86) PCT No.: PCT/EP2022/074773

§ 371 Date: Feb. 29, 2024

(30) Foreign Application Priority Data
Sep. 6, 2021 (EP) 21195141.3

(51) Int. Cl. G16H 50/20; G16B 5/20; G16B 20/20; G16B 30/10; G16B 40/20

(52) U.S. Cl. CPC G16H 50/20; G16B 5/20; G16B 20/20; G16B 30/10; G16B 40/20

(57) ABSTRACT

The present invention relates to a method for the diagnosis and/or classification of a disease in a subject based on the genetic and/or epigenetic information of a sample obtained from the subject, the method comprising the steps of: a) providing data from said sample, wherein said data comprises genetic and/or epigenetic information of a random subset of genomic positions: b) assigning said sample to a sample class based on genetic and/or epigenetic information of said random subset of genomic positions by employing a computational model, which discriminates a plurality of sample classes based on genetic and/or epigenetic information of a set of genomic positions comprising said random subset, wherein the computational model has been trained with pre-determined genetic and/or epigenetic information obtained from a plurality of pre-classified samples of known diseases and wherein said computational model processes the genetic and/or epigenetic information of a genomic position of said random subset independently of the genetic and/or epigenetic information of another genomic position of said random subset, wherein said computational model is preferably in the form of a linear classifier with independent feature sampling.

The present invention relates to a method for the diagnosis and/or classification of a disease in a subject based on the genetic and/or epigenetic information of a sample obtained from the subject, the method comprising the steps of: a) providing data from said sample, wherein said data comprises genetic and/or epigenetic information of a random subset of genomic positions; b) assigning said sample to a sample class based on genetic and/or epigenetic information of said random subset of genomic positions by employing a computational model, which discriminates a plurality of sample classes based on genetic and/or epigenetic information of a set of genomic positions comprising said random subset, wherein the computational model has been trained with pre-determined genetic and/or epigenetic information obtained from a plurality of pre-classified samples of known diseases and wherein said computational model processes the genetic and/or epigenetic information of a genomic position of said random subset independently of the genetic and/or epigenetic information of another genomic position of said random subset, wherein said computational model is preferably in the form of a linear classifier with independent feature sampling.

In this specification, a number of documents are cited. The disclosure of these documents, while not considered relevant for the patentability of this invention is herewith incorporated by reference in its entirety. More specifically, all referenced documents are incorporated by reference to the same extent as if each individual document was specifically and individually indicated to be incorporated by reference.

Several diseases have been shown to be associated with abnormalities in the genome, such as mutations in a single gene, chromosomal abnormalities or differential DNA methylation. Thus, molecular classifiers are a helpful tool for the identification of diseases that are associated with specific genetic and/or epigenetic patterns, such as e.g. congenital disorders like Fragile X syndrome (Monk et al. 2016), Down syndrome (Aref-Eshgi et al, 2020) or Imprinting Disorders (Monk et al., 2016). Furthermore, they even allow the differentiation of specific tumor species based on e.g. genome-wide methylation profiles. Also, it has been demonstrated that cardiovascular disorders such as myocardial infarction can be detected in peripheral serum samples by DNA methylation analysis of DNA fragments contained in the cell free fraction of the blood sample (Zemmour et al., 2018, Cuadrat et cl., 2021). Further, infectious disorders such as acute respiratory infections with SARS-CoV-2 (Severe acute respiratory syndrome coronavirus type 2) and the subsequent course of the disease leading to either hospitalization and/or admission to an intensive care unit and/or death can be predicted from genome-wide DNA methylation profiles (Konigsberg et al., 2021). Additionally, it has been demonstrated previously that CpG methylation patterns can be used to distinguish patients with rheumatoid arthritis from unaffected individuals (Liu et al., 2013).

Despite the recent technological advances, the diagnosis and classification of such diseases remain a significant challenge in the clinical setting. This is because the practice of medical genetics and epigenetics is often time-consuming, involving the steps of sample preparation (including DNA isolation, DNA library preparation), as well as data acquisition and the analysis of complex datasets, especially as a result of new technologies such as next-generation sequencing (NGS).

A specifically challenging field is the intraoperative diagnosis of primary and secondary brain tumors (Hollon et al., 2020 and Djirackor et al., 2021).

Current best clinical practice consists of identifying brain tumors with non-surgical treatment such as CNS lymphomas from those preferably treated with cytoreductive surgery such as gliomas through intraoperative frozen section diagnostics. Currently, no generalizable molecular approach towards the intraoperative diagnosis of a comprehensive number of CNS tumor entities exists. Recently, based on genome wide DNA methylation patterns, molecular classifiers for primary CNS tumors, intracerebral metastasis as well as sarcomas haven been established (Capper et al., 2018, Orozco et al., 2018, Koelsche et al., 2021). Conventional methods for the genome-wide assessment of CpG methylation and prognostic molecular features in gliomas remain labor intensive and time-consuming and cannot deliver diagnostic feedback within the timeframe of a routine neurosurgical procedure.

Nanopore sequencing is an emerging technology offering the opportunity to examine the genetic and/or epigenetic information of a sample as e.g. the DNA methylation information of a tumor sample on a handheld device only milliseconds after a DNA segment has passed through a nanopore (Simpson et al., 2017). Yet, the diagnostic interpretation of nanopore sequencing datasets in real-time has so far not been possible.

Recently published classification methods from DNA methylation data rely on information from a fixed number of genomic positions, for example, as obtained from Illumina Infinium HumanMethylation450 or EPIC BeadChip arrays. However, when applied to real-time sequencing as e.g. to nanopore sequencing, this requirement is not fulfilled as only sparse, shallow coverage data is available.

In view of the labor intensive and time-consuming approaches for the diagnosis and/or classification of a disease in a subject, there is an urgent need in the art to overcome this problem. This need is addressed by the present invention.

Thus, the present invention relates to a method for the diagnosis and/or classification of a disease in a subject based on the genetic and/or epigenetic information of a sample obtained from the subject, the method comprising the steps of: a) providing data from said sample, wherein said data comprises genetic and/or epigenetic information of a random subset of genomic positions; b) assigning said sample to a sample class based on genetic and/or epigenetic information of said random subset of genomic positions by employing a computational model, which discriminates a plurality of sample classes based on genetic and/or epigenetic information of a set of genomic positions comprising said random subset, wherein the computational model has been trained with pre-determined genetic and/or epigenetic information obtained from a plurality of pre-classified samples of known diseases and wherein said computational model processes the genetic and/or epigenetic information of a genomic position of said random subset independently of the genetic and/or epigenetic information of another genomic position of said random subset, wherein said computational model is preferably in the form of a linear classifier with independent feature sampling.

The term “disease” as used herein is used interchangeably with the terms “disorder”, “condition” (as in medical condition) and “illness”, in that all reflect an impairment of health and/or an abnormal condition of the human or animal body or of one of its parts that impairs normal functioning. Accordingly, the term “disease” may e.g. refer to a disease characterized by dysregulated organ development, abnormal cell functioning, proliferation and/or growth. Examples of diseases are developmental disorders, such as Fragile X syndrome, Imprinting disorders or inborn errors of metabolism, cardiovascular disorders such as stroke, ischemic heart disease, peripheral artery occluding disease, disorders of the endocrine system such as diabetes and hypothyroidism, autoimmune disorders such as rheumatoid arthritis or multiple sclerosis, infectious disorders such as septicemia, neonatal sepsis, coronavirus disease—19 (COVID19) and malaria, neurodegenerative disorders such as Alzheimer's disease or Parkinson's Disease and mental disorders such as major depression or schizophrenia.

In a preferred embodiment of the invention, the disease is cancer.

As used herein, the term “cancer” refers to a disease characterized by dysregulated cell proliferation and/or growth. Examples of cancers include, but are not limited to brain tumors, blastoma, carcinoma, leukemia, lymphoma, melanoma, squamous-cell skin cancer, basal-cell skin cancer, multiple myeloma, sarcoma, ductal breast cancer, lobular small-cell lung cancer, non-small-cell lung cancer, prostate cancer, primary hepatic cancer, colon cancer, gastric cancer, cervical cancer, ovarial cancer and other cancer types (for other cancer types see WHO Classification of Tumours Series, https://publications.iarc.fr/Book-And-Report-Series/Who-Classification-Of-Tumours).

The term “cancer” as used herein is not limited to any stage, grade, histomorphological feature, invasiveness, aggressiveness or malignancy of an affected tissue or cell aggregation. In particular stage 0 cancer, stage I cancer, stage II cancer, stage III cancer, stage IV cancer, grade I cancer, grade II cancer, grade III cancer, malignant cancer, primary carcinomas, and all other types of cancers, malignancies etc. are included.

In a particularly preferred embodiment of the invention, the disease is a brain cancer.

The term “diagnosis and/or classification of a disease in a subject” as used herein refers to the identification of the incidence of a disease in a subject, as well as the categorization, classification, differentiation, grading, staging or prognosis of a disease. Accordingly, as shown in the examples herein below, the method of the invention can be used for the classification of a tumor into a particular tumor class. The term “tumor class” or “tumor species” as used herein refers to a specific kind of a tumor or sub-category of a tumor that can be classified based on its tissue origin, genetic make-up, histology etc.

The term “subject” as used herein refers to a vertebrate, preferably a mammal. Human subjects are most preferred.

It is understood that a disease in accordance with the present invention is characterized by specific genetic and/or epigenetic patterns. Accordingly, the diagnosis and/or classification of a disease in a subject is made based on the genetic and/or epigenetic information of a sample obtained from the subject.

The term “genetic information” as used herein refers to information on the nucleotide composition of a nucleic acid, i.e. the nucleotide sequence information of a nucleic acid (i.e. the primary sequence information).

The term “epigenetic information” as used herein refers to information other than the primary sequence information of a nucleic acid and encompasses information about changes to genomic DNA and/or proteins associated with genomic DNA that are stably inherited through cell division. Epigenetic information includes, but is not limited to, e.g. information on DNA methylation, histone methylation, histone deacetylation and open and closed chromatin patterns (e.g. as measured with methods for determining transposase accessible genomic regions or adenine-methylase accessible genomic regions).

It is important to note in this context that epigenetic information pertaining to as outlined can be inferred from patterns that can be observed from a plurality of cell-free DNA (cfDNA) fragments. Such cfDNA fragments can be detected in serum, saliva, blood or cerebrospinal fluid. These cfDNA fragments are of particular interest for this invention, because they can contain not only sequence and CpG methylation patterns, but also the fragment position and size can contain specific information in relationship to the epigenetic state at the genomic position of the cells they are derived from (Katsman et al., 2022).

As one illustrating example for the implementation of the present invention towards detecting diseases and establishing e medical diagnosis, one may consider a situation, where one wishes to distinguish two different malignancies with the present invention in cfDNA samples from each other: For this one may consider two adjacent genomic positions, named in this example position A and position B. Nanopore sequencing of the cfDNA from two patients with different malignancies are named for this example malignancy X and malignancy Y. The results reveal in this example many cfDNA reads mapping in the case malignancy X on genomic position A, but only a few reads mapping to the other genomic position B. The reverse may be true in the other patient suffering from malignancy Y: Few reads map to position A but many to B in the case of malignancy Y.

The term “mapping” in the context of the present invention relates to connecting two concepts with each other based on but not limited to statistical, categorical or classification-based associations. One example is mapping a read to a genomic position based on the similarity of the nucleotide sequence of the genomic position and the nucleotide sequence of the sequencing read obtained from a DNA sample processed in a DNA sequencing apparatus. Another example is the mapping of categorical classes such as medical diagnoses or classification systems of medical diagnosis on groups of samples or on one sample from an individual patient.

One explanation for the observation of differentially mapping results between malignancy X and Y in relationship to genomic positions A and B can be that most reads mapping to genomic position A in cancer X originating from a distinct cell type, e.g., a glial cell. In this cell type, genomic position A may be epigenetically accessible to regulatory proteins such as transcription factors. Genomic position A may be inaccessible in another cancer originating from a different cell type, e.g., a lymphoid cell and thus may be epigenetically inaccessible to regulatory proteins such as transcription factors. This differential epigenetic state regarding position A and B between two malignancies X and Y then in turn may influence characteristically the cfDNA's fragment size, position and fragment end motifs, as open and closed chromatin have been demonstrated to show differential fragmentation patterns in cfDNA nanopore datasets (Katsman et al., 2022). The computational model of the present invention can be trained and compute sample class probabilities based on such genomic position specific measurement as detailed herein below.

Also, the plurality of cfDNA reads derived from a patient suffering from a tumor may contain information on copy number variations specific to the said tumor which may be also characteristic for the sample class said tumor belongs to (Katsman et al., 2022). Similarly, the computational model of the present invention described herein below can be trained and compute sample class probabilities based on such genomic position specific measurement as detailed herein below.

Accordingly, in a preferred embodiment of the present invention, the genetic and/or epigenetic information comprises information about: a) DNA methylation, b) single nucleotide polymorphisms, c) histone modifications, c) structural variations such as deletions, insertions, inversions, tandem repeat variations, substitutions, disruptions, or d) copy number variations, chromosomal losses or super numerous chromosomes with respect to a reference genome.

Such genetic and/or epigenetic information can be observed directly through sequencing assays such as ATAC-seq or determination of CpG methylation through bisulfite or nanopore sequencing.

In an alternative embodiment, such genetic and/or epigenetic information may be inferred indirectly through cfDNA positional fragmentation patterns, fragmentation size patterns and/or fragment end motifs.

The term “single nucleotide polymorphism (SNP)” as used herein refers to a variation of a single base pair in a complementary DNA double strand with respect to a reference genome. SNPs are inherited and heritable genetic variants. Consequently, many SNPs and other structural variations have been catalogued in population-based sequencing projects and can be accessed in databases (e.g. at the gnomAD database; https://gnomad.broadinstitute.org). SNPs can indicate differences in the susceptibility of human subjects to a wide range of diseases (e.g. sickle-cell anemia, β-thalassemia and cystic fibrosis). The term “mutation” usually refers to a newly occurring change with respect to a reference genome and can be considered a hallmark of certain cancer types. The severity of illness and the way the body responds to treatments are also manifestations of genetic variations caused by SNPs. Some single base pair mutations have been shown to be characteristic for certain brain tumors and may e.g. occur in the IDH1/2 gene or the TERT gene.

The term “structural variation” as used herein is the variation in structure of an organism's chromosomes which are greater than 50 DNA base pairs. Structural variations consist of many kinds of variation in the genome of one species, such as deletions, duplications, copy number variants, insertions, inversions and translocations. Structural variations as used herein are to be distinguished from SNPs. Structural variations can e.g. be present in a genome of normal somatic tissues of a human individual not affected by cancerous transformation, but structural variations can also be cancer related genomic mutations and then only be present in the cancerous tissue. The term “mutation” usually refers to a newly occurring change with respect to a reference genome and can be considered a hallmark of certain cancer types.

Structural variations can occur at any genomic scale such as deletions, insertions, inversions, tandem repeat variations, substitutions, disruptions and can also include genomic variations involving whole or partial chromosomes.

In another example, chromosomal mutations characteristic for specific brain tumors such as oligodendrogliomas occur at chromosomal arms 1p and 19q. Similarly, structural mutations characteristic for specific high-grade brain tumors occur as loss of the CDKN2A/B genes.

The structural variations in accordance with the preferred embodiment described above include small variations of more than 50 bases and large variations of above 50 base pairs.

For the determination of the characteristic genetic and/or epigenetic information (i.e. also referred to herein as genetic and/or epigenetic variations) described in the preferred embodiment above, it is important to note that these variations are determined by comparing DNA sequence information from a sample obtained from the subject with a reference genome.

The term “reference genome” as used herein refers to a digital representation of the DNA sequence from an organism of the respective species. The currently most widely used human reference genome is termed GRCH38.p13 from the Genome Reference Consortium of the NCB1 reference database. GRCH38.p13 not only provides most of the nucleotide sequence encompassing the 23 Chromosomes and mitochondrial genome of an idealized human individual, but also assigns a unique location (position) to most of the nucleotide sequences that can be retrieved through sequencing of a human genome. The location is encoded in a coordinate system first identifying the chromosome number a sequence is located on (e.g. “chr1” for a nucleotide located on chromosome 1) and then stating with a number for the sequential position where a nucleotide can be found on a chromosome. Accordingly, chr11:2,023,002 refers to the nucleotide cytosine which can be found on the position 2,023,002 on Chromosome 11.

In the context of this invention a reference genome is a) a prerequisite to determine above mentioned genomic variants and b) a reference genome allows to position a genomic variant into the coordinate system of the reference genome. The present invention is not limited to a linear representation of a reference genome as it is the case with the GRCH38.p13 reference but can be also applied to other coordinate and symbolic systems suitable for the representation of reference genomes such as pangenome graphs (Li et al., 2020).

In a particularly preferred embodiment, the genetic and/or epigenetic information comprises information on the methylation status of CpG nucleotides.

Cytosines in CpG dinucleotides can be methylated to form 5-methylcytosine. Methylation of (i.e., introduction of a methyl group) the cytosines of CpG site within the promoters of genes can lead to gene silencing, an epigenetic modification (i.e. epigenetic feature) found in a number of human cancers. In contrast, the hypomethylation of CpG sites has generally been associated with the overexpression of oncogenes within cancer cells.

The term “methylation status”, as used herein describes the state of methylation of a CpG position, thus refers to the presence or absence of 5-methylcytosine at one CpG site within genomic DNA. As used herein the term “CpG site” or “CpG position” refers to a region of DNA where a cytosine nucleotide occurs next to a guanine nucleotide in the linear sequence of bases along its length, the 5′-cytosine (C) being separated by only one phosphate (p) from the 3′-guanine (G).

The term “sample” as used herein refers to a biological sample. The sample may be of any biological tissue or fluid. Samples in accordance with the present invention include, but are not limited to, tissue, cerebrospinal fluid, blood, serum, plasma, blood cells, urine, stool, peritoneal fluid and pleural fluid, core or fine needle biopsy samples, sputum or cell-containing body fluids. From such a sample DNA from cells contained in the sample but also cfDNA may be extracted and subsequently analyzed with a sequencing apparatus. For the epigenetic and/or genetic analysis from such a sample, DNA may be extracted for being analyzed e.g., with a sequencing apparatus to obtain genetic and/or epigenetic information. Such DNA can be obtained from the cells contained in the sample and from the non-cell containing component of the sample.

In a preferred embodiment of the invention, the sample is a tumor sample.

In a particularly preferred embodiment of the invention, the sample is a brain tumor sample.

Generally, a sample in accordance with the present invention comprises nucleic acid derived from genomic DNA. As used herein, the term “nucleic acid”, “nucleotide sequence” or “polynucleotide” refers to the arrangement of either deoxyribonucleotide or ribonucleotide residues in a polymer in either single- or double-stranded form of any length. Nucleic acid sequences can be composed of natural nucleotides of the following bases: T, A, C, G, and U, and/or synthetic analogs of the natural nucleotides. In the context of the present invention, adenosine is abbreviated as “A”, cytidine is abbreviated as “C”, guanosine is abbreviated as “G”, thymidine is abbreviated as “T”, and uridine is abbreviated as “U”. A polynucleotide can be a single-stranded or a double-stranded nucleic acid. The term “polynucleotide” as used herein relates to a nucleic acid sequence of any length. The term “genomic DNA” as used herein refers to chromosomal and mitochondrial DNA.

In a preferred embodiment of the invention, the sample is a tissue sample.

In another preferred embodiment of the invention, the sample is cfDNA from a liquid biopsy.

In accordance with a particularly preferred embodiment, the amount of tissue sample is at least 0.5 mg, preferably at least 1 mg, more preferably at least 2 mg, more preferably at least 5 mg and most preferably at least 20 mg.

In accordance with a particularly preferred embodiment, the amount of a liquid biopsy sample obtained from a blood or cerebro-spinal fluid withdrawal is at least 0.1 ml, preferably at least 1 ml, more preferably at least 2 ml and most preferably at least 5 ml.

In another preferred embodiment of the invention, the sample has been obtained intraoperatively.

The method for the diagnosis and/or classification of a disease in a subject according to the present invention comprises a step of a) providing data from said sample, wherein said data comprises genetic and/or epigenetic information of a random subset of genomic positions.

Accordingly, the genetic and/or epigenetic information of a sample obtained from the subject comprises genetic and/or epigenetic information of a random subset of genomic positions within the genomic DNA of said sample.

As outlined above, the genetic and/or epigenetic information from a sample is evaluated by sequencing a sample and comparing the obtained information with that of a reference genome. It is understood by the skilled person in the art that a reference genome consists of sequence information of chromosomal as well as mitochondrial DNA of an idealized individual. Consequently, also variations in the mitochondrial DNA sequence can be assessed by the method of the invention.

The term “genomic position” in accordance with the present invention relates to a position within genomic DNA, including mitochondrial DNA. A “genomic position” in accordance with the present invention encompasses, but is not limited to a single nucleotide, a nucleotide sequence, a gene, a genomic region with protein-coding RNA transcription such as an exon of an gene, a genomic region with non-protein-coding function such as a long or short non-coding RNA transcription, a microRNA gene, a regulatory genomic region such as a promotor region, a regulatory genomic region such as an enhancer region, a regulatory genomic region such as a silencer region, a genomic region such as a topologically associated domain (TAD), a boundary of a topologically associated domain (TAD), a repetitive element such as a micro- and minisatellite, a satellite array, a retrotransposon such as a long interspersed nuclear element (LINE) or a long terminal repeats (LTR) or a short interspersed nuclear element (SINE), a transposable region, such as an ALU element, a centromeric repeat, a higher order centromeric repeat (HOR), a telomeric repeat, a segmental duplication, a ribosomal rRNA array, a p- or q-arm of an chromosome, a chromosome in its entirety, and any region of a genome, as e.g. a human genome. Structural variations in an individual can occur at any of the listed genomic positions and can alter the function of any of those classes of genomic positions.

Hence, the present invention allows to accurately diagnose and/or classify, for example, brain tumors by determining e.g., the methylation status (i.e. the epigenetic information) of only a few CpG dinucleotides (i.e. subset of genomic positions) sequenced in random order in the context of an entire human genome.

The method for the diagnosis and/or classification of a disease in a subject according to the present invention further comprises the step of b) assigning said sample to a sample class based on genetic and/or epigenetic information of said random subset of genomic positions by employing a computational model, preferably in the form of a linear classifier with independent feature sampling, which discriminates a plurality of sample classes based on genetic and/or epigenetic information of a set of genomic positions comprising said random subset, wherein the computational model has been trained with pre-determined genetic and/or epigenetic information obtained from a plurality of pre-classified samples of known diseases and wherein said computational model processes the genetic and/or epigenetic information of a genomic position of said random subset independently of the genetic and/or epigenetic information of another genomic position of said random subset.

The term “sample class” as used herein refers to samples which have been assigned to a group (i.e., a class) based on quantitative information on one or more characteristics inherent in the samples (referred to as traits, variables, characters, features etc.).

Accordingly, a “sample class” in accordance with the present invention may represent a specific kind of a disease or a sub-category of a disease that can be distinguished from other kinds of diseases or subcategories of diseases based on its genetic and/or epigenetic information. Such a sample class may be consistent with a disease class from a disease classification system. For example, in the field of brain tumors, various distinct brain tumor species or classes can be discriminated based on their genome wide methylation pattern (Capper D et al., 2018) but also is a class in the 2021 WHO Classification of Brain Tumors (Louis et al, 2021).

The term “assigning said sample to a sample class” refers to a procedure in which the individual samples are placed into sample classes based on their genetic and/or epigenetic information. Assignment in accordance with the present invention is achieved by employing the computational model, preferably in the form of a linear classifier with independent feature sampling, used in the method of the invention. Said assignment to a sample class, which may correspond to a disease class of a disease classification system such as for example the 2021 WHO Classification of Brain Tumors (Louis et al, 2021), will lead to the assignment of a diagnosis to said sample from an individual patient.

Accordingly, a “sample class” as assigned by the method of the present invention may correspond to a disease class from an existing classification system but can also relate to subgroups of existing disease classes with features that can be distinguished based on molecular features. Such subclasses in the form of sample classes can be utilized by those knowledgeable in the art of medicine as diagnosis to further select optimal treatments.

For example, the term “glioblastoma” refers to a disease class in the 2021 WHO Classification of Brain Tumors with no further subcategorization. Based on methylation profiling, several subcategories of glioblastoma can be identified (Capper et al., 2018). Among those are for example Receptor tyrosine Kinase (RTK) I and II and mesenchymal glioblastoma disease subclasses, which all fall under the 2021 WHO Classification of Brain Tumors disease class “glioblastoma”.

Similarly, the glioblastoma subclass RTK II glioblastomas have been demonstrated to show a significantly higher incidence of seizures than RTK I and mesenchymal (MES) glioblastoma (Ricklefs et al., 2022). Consequently, a person skilled in the art of medicine can utilize a sample class assignment from a patient derived tumor sample by the present invention to the glioblastoma RTK I disease class to diagnose a glioblastoma in said patient as well as utilize the assignment of said sample to the RTK I glioblastoma disease class as medical diagnosis to subsequently initiate an antiepileptic treatment in the form of an antiepileptic medication to the benefit of said patient without inventive skill and undue burden.

As an additional example, there is evidence that patients suffering from a RTK I or RTK II glioblastoma may benefit in their survival from more extensive neurosurgical resections (Drexler et al, 2022). Therefore, identification of the subclasses RTK I or II in a patient intraoperatively with the present invention leads to a rapid diagnosis and subsequent differential therapeutic treatments to the benefit od said patient.

The term “computational model” as used herein generally refers to a computational process (associated with computer programming or other written instructions) involved in transforming information from one state to another.

The term “computational model in the form of a linear classifier with independent feature sampling” in the context of the present invention encompasses a group of classification methods, which can be utilized in the form of computational models. Among those computational models, the Naïve Bayes classifier is an example. In a particularly preferred embodiment, the computational model in the form of a linear classifier with independent feature sampling is a Naïve Bayes classifier model. Further examples include logistic regression models or linear support vector machine models. Accordingly, at variation with the above particularly preferred embodiment, the computational model in the form of a linear classifier with independent feature sampling is a logistic regression model or a linear support vector machine model. More generally, any method, which is a linear function of features or single feature functions with a linear decision boundary can be replaced by an appropriately defined Naïve Bayes classifier and is hereby included in the definition as Naïve Bayes classifier; see also James et al., 2021). Another term for such a computational model in the form of a linear classifier with independent feature sampling used in the literature is “generalized additive model” (James, Witten, Hastie, Tibshirani, 2021).

In the context of this invention, it also understood that so called “deep learning algorithms” can be used to replicate the function of any linear computational model such as described in the present invention. Therefore, it is understood that the present invention encompasses also all methods using artificial neural networks if such an artificial neural network replicates and/or approximates any computational model in the form of a linear classifier with independent feature sampling.

In accordance with step b) of the method of the present invention, pre-determined genetic and/or epigenetic information obtained from a plurality of pre-classified samples of known diseases is used for training the computational model.

The term “training” as used herein relates to the determination of parameters for each genomic position for which genetic and/or epigenetic information is available by the computational model from pre-classified samples. The genomic position-specific parameters include but are not limited to the sample class-specific probability, expected value and variance of observing a sample class-specific genetic and/or epigenetic information at a specific genomic position and sample class-specific prior probabilities and position-specific technical parameters, such as measurement error. Such training of the computational model may be performed by any suitable means and methods known in the art.

In order to train the computational model, sample class-specific, genomic position-specific rates (e.g. methylation rates) for categorical variables encoding genetic and/or epigenetic information (e.g. binary or non-binary variables) need to be provided to the computational model.

The term “methylation rate” as used herein refers to a representation of a CpG position specific categorical variable representing the rate at which a CpG is found to be methylated by a measurement of a plurality of DNA fragments from said position from a sample. For example, Illumina Methylation Arrays report the methylation rate in form of a β-value between 0 (never found to be methylated) and 1 (always found to be methylated) in a sample or sample class. As example, a β-value of 0.4 indicates that a CpG is found to be methylated in approximately 40% of all measurements in said specific sample.

The term “event rate” as used herein represents the generalization of the term methylation rate to any means for the determination of position specific genetic and/or epigenetic information known in the art. Such information includes but is not limited on DNA methylation, histone methylation, histone deacetylation and open and closed chromatin, single nucleotide polymorphisms, histone modifications, structural variations such as deletions, insertions, inversions, tandem repeat variations, substitutions, disruptions, or copy number variations, chromosomal losses or super numerous chromosomes with respect to a reference genome, including but not limited to position specific fragment size, position specific fragment frequencies and fragment end motifs in cfDNA.

The term “pre-classified samples” relates to samples that were classified by prior art methods and therefore are of known diseases.

As shown in the examples herein below, the present invention allows the real-time assessment of genetic and/or epigenetic data streams, as e.g. obtained by nanopore sequencing and hence allows the clinical interpretation of sequencing datasets in real time. In this context, even if only a random subset of genetic and/or epigenetic information on a sample obtained from a subject is available, extremely precise predictions can be made about the concordance with a disease in said subject. The present invention is universally applicable and can be used for all known diseases as defined herein above.

Furthermore, the method of the invention uses a stable, predefined computational model and there is no need to retrain the model for each sample to be diagnosed. The term “stable” with respect to the computational model of the invention refers to the quality of the computational model insofar that said computational model does not require any modification, updating or retraining for the purpose of assigning a sample to a sample class (step b) of the method of the invention), e.g. for the classification of a tumor to a particular tumor class. Regulatory evaluation and approval of the computational model of the invention in its entirety for CE-IVD (CE-marked In Vitro Diagnostic Device) products is thus possible and no approval of additional algorithmic procedures for modification, updating or retraining of said computational model are required for regulatory approval.

Competing methods such as described by Kuschel et al., 2021 and Dijtractor et al 2021 are based solely on retraining a random forest computational model for each subset of CpG methylation information obtained by nanopore sequencing, thus making it impossible to submit and evaluate the random forest classification model for regulatory evaluation before the diagnostic procedure is performed.

Furthermore, step b) of the method of the present invention does not limit the analysis to a predefined set of genomic positions. For example, the DNA methylation-based approach for CNS tumor diagnostics described in Capper et al., 2018 includes a limitation of the analysis to a predefined set of CpG positions, i.e. “CpG markers” and thus includes a feature selection step. The term “feature selection” generally refers to a process of selecting a subset of relevant features (variables, predictors) for use in model construction. In contrast, the method of the present invention does not require a feature selection step, as the computational model used in step b) of the method of the present invention allows the assignment of a sample obtained from the subject to one of the sample classes based on the genetic and/or epigenetic information of a random subset of genomic positions.

The method of the invention also does not rely on any means on biological and/or computational feature identification and/or selection in contrast to competing methods.

Such feature selection steps are also referred to in the literature as “marker selection” “marker identification”, “signature identification”, “featurization” or any combination of the latter.

Such selected limited subsets of genetic and/or epigenetic information (i.e. genetic and/or epigenetic information obtained from a specific subset of genomic positions) are also referred to in the literature as “signatures”, “feature sets”, “feature selected subsets”, “markers”, “biological markers”, “marker sets” or any combination of the latter.

For competing methods, such feature-selected subsets of genetic and/or epigenetic information are then converted into diagnostic methods assessing said feature selected subsets in a sample. These diagnostic methods for assessment of feature-selected subsets of genetic and/or epigenetic information are referred to in the literature as “markers” or “marker panels” or “diagnostic markers” or “diagnostic features”.

In contrast to the method of the invention, competing methods e.g as described in WO2016106391A1 for predicting tumor classes intraoperatively critically rely on a small subset (less than 10 features) of genetic and/or epigenetic information, which have to be defined a priori and also feature-specific molecular tests such as optimized polymerase chain reactions have to be established before the diagnostic procedure is initiated.

Other competing methods such as e.g. those described in U.S. Pat. No. 9,984,201B2 or WO20180203974A1 or WO2020232109A1 for predicting tumor classes, in contrast to the method of the invention, critically rely on the computational selection of a subset of features with the most significant predictive value by a scoring mechanism in the training step and then can only consider the feature selected subset for the prediction of a sample class from a sample based on genetic and/or epigenetic information on the feature selected subset.

In the context of brain tumor classification as described in the examples herein below, this means that all CpG positions (i.e. genomic positions of the random subset) for which a methylation call (i.e. genetic and/or epigenetic information) is available can be included in the analysis, given that the methylation of the same CpG position has been included in the training of the computational model, i.e. has been included in the set of genomic positions. Consequently, the method of the present invention does not require a feature selection step in the sense that it focuses on a set of genomic positions based on e.g. filtering for a subset of genomic positions with the highest variability or selecting those genomic positions carrying the most predictive value. Accordingly, the method of the present invention does not require a specific order at which the genomic positions are processed. Furthermore, the method of the invention does not require the imputation of missing values, as any subset of genomic positions for which genetic and/or epigenetic information is available can be processed.

Competing methods for prediction of cancers from cfDNA such as those described by Bie et al. 2022 trained random forest classifier on a set of machine learning algorithms utilized on genetic and/or epigenetic information obtained from cfDNA, which was extracted from serum of cancer patients suffering from early- and advanced-stage cancers of the breast, colorectum, esophagus, stomach, liver, lung, or pancreas.

Genetic and/or epigenetic information obtained from cfDNA included DNA methylation, position specific cfDNA fragment size, copy number variation and fragment end motifs. Particularly CpG methylation and fragment size and fragment end motifs have been demonstrated in the literature to be correlated with cancers' tissue origin and reflected tissue specific accessible regulatory genomic positions.

Also in the case of analysis of complex genetic and/or epigenetic information observed from cfDNA, the method of the invention is not limited to a preselected subset of features or requires the use of dimension reduction methods such as PCA. Additionally, the method of the invention integrates diverse sources of genetic and/or epigenetic information such as CpG methylation and copy number variation profiles within the same computational model in the form of a linear classifier with independent feature sampling and does not require the simultaneous utilization of a diverse set of machine learning methods such as Principal Component Analysis, Support Vector Machines, Logistic Regression models and Random Forrest models as it is the case with competing methods such as presented by Bie at al. 2022.

Thus, the method of the present invention allows the diagnosis and/or classification of a disease in a subject, even if the data provided in step a) comprises genetic and/or epigenetic information of only a small, randomly provided number of genomic positions (i.e. even if only sparse, shallow coverage data is available).

The term “diagnosis” is commonly understood as the identification of the nature and cause of a certain phenomenon. A diagnosis in the medical context is typically used to determine the causes of symptoms and is most importantly intended to deduct mitigations and solutions based on the current medical best practices relating to the specific diagnosis.

As an illustrating and non-limiting example, a patient is admitted to the emergency room with an unprovoked cerebral seizure. Imaging studies reveal an intracerebral tumor mass of unknown nature. After open craniotomy an oligodendroglioma is diagnosed in the patient with current clinical best practice methods from a tumor-derived biopsy results including microscopic neuro-histopathological assessment of section of the biopsy, the determination of the IDH1/2 mutation status and confirmation of the loss of chromosome arms 1p/19q. The described procedure for the diagnosis of said oligendroglioma in a patient is outlined in the 2021 WHO Classification of Brain Tumors (Louis et al, 2021). In this example, a person skilled in the art of medicine uses the class suggested by the diagnostic procedures and the 2021 WHO classification as diagnosis to determine the further course of action to treat said patient.

From the point of view of statistics, diagnostic procedures involve classification tests determining the class of a disease entity with a probability from a catalogue of possible classes. Such diagnostic classes are then used to deduct information on ways to initiate mitigations and solutions of such a health problem.

In the illustrating example, the diagnosis of an oligodendroglioma suggests, based on current treatment guidelines, the resection of the tumor and specific chemotherapeutic agents and radiotherapy.

The method of the present invention determines from a multitude of predefined sample classes a sample class with a probability for said sample class based on genetic and/or epigenetic information subjected to a computational model.

In the above outlined illustrating example, the present invention can determine from a tumor derived biopsy based on genetic and/or epigenetic information from said tumor biopsy the class of the patient's tumor with an associated probability—in this case an oligodendroglioma from a computational model as outlined in Example 5 provided with the present invention. In the illustrating example the sample class assigned by the present invention “oligodendroglioma” to the tumor sample is identical to the diagnosis “oligodendroglioma” leading to the clinical best practice treatment of the disorder. A person skilled in the art of medicine will employ the sample class with its associated probability determined by the present invention as diagnosis.

As further outlined herein, the present invention can determine sample classes based on genetic and/or epigenetic information from a patient sample not only in the area of the preferred embodiment of the present invention, i.e., diagnosis of CNS malignancies. Said mapping of sample classes onto medical diagnosis can be aided by choosing sample classes for training the computational model that are identical with or overlap with or represent subclasses of medical diagnosis as they are described and defined in medical classification systems such as the 2021 WHO Classification of Brain Tumors (Louis et al, 2021) or the WHO International Classification of Diseases 11 (WHO ICD-11).

In another non-limiting example of the present invention, a computational model which has been trained on e.g. data from blood from patients with COVID-Infections, other respiratory problems and controls can assign samples to the three sample classes and thus provide actionable diagnostic information to persons skilled in the art of medicine without undue burden and without requiring inventive skill.

In a preferred embodiment of the present invention, the random subset of genomic positions consists of at least 50, preferably at least 100, more preferably at least 200, more preferably at least 500, more preferably at least 1000 and most preferably at least 2000 genomic positions.

In contrast, the computational model used in the method described by Capper et al. 2018 requires information from all measured approximately 450,000 CpG sites on an Illumina Methylation Array where the most variable CpG sites are being selected (approximately 20,000-40,000) and used for the random forest classification; see WO 2016/142533. In a recently published variant (Kuschel et al., 2021) of the procedure a subset of approximately 1,000-50,000 CpGs are sampled and based on these CpG the random forest classifier is being retrained using the epigenetic information of the training dataset of all 450,000 CpGs on an Illumina methylation array. The principal difference between the competing methods and the present invention is that the subset of CpGs needs to be known before training for the competing methods, while the present invention does not require the subset to be defined before training the computational model.

Furthermore, the computational model used in the method of the present invention processes the genetic and/or epigenetic information of a genomic position of said random subset independently of the genetic and/or epigenetic information of another genomic position of said random subset. Hence, unlike competing methods, as e.g. described in Capper et al., 2018, the method of the invention employs no interaction model of genetic and/or epigenetic information from different genomic positions. In contrast, computational models which are based on random forest classification employ so-called “decision trees” or “classification trees” that place categorical variables into classes.

To further disclose the use of one genomic position independently of another genomic position from a random subset to derive one class assignment result, one may consider the network depiction (FIG. 1) of a naïve Bayes classifier as example for a generalized linear model: in FIG. 1a a textbook depiction of a Naïve Bayes Classifier along with its formula is displayed. In FIG. 1b, the depiction is modified with elements of the present invention. Genetic and/or epigenetic information each from distinct genomic positions are introduced into a computational model for deriving the probability for the presence of a specific disease class. The trained computational model is represented in this figure through the arrows leading from the computation of a specific disease class (represented by the circle in the middle), depending on the conditional genetic and or epigenetic information from said 1, 2 . . . n genomic positions integrating the diverse measurements into a probability estimate. Note that the probability computation of any number of disease classes can be performed in parallel and the number of disease classes is only limited by the number of computational models trained on existing datasets with samples representing each disease class. Note also that FIGS. 1a and 1b illustrate only the computation of one class/disease probability. Any number of class/disease probability can be computed in parallel, if alternative computational models have been trained on genetic and/or epigenetic data from other sample classes.

Importantly, each genetic and/or epigenetic information from the sample measurements is processed in this computational model independently, which is indicated by the lack of any connections in between the four genetic and/or epigenetic information from distinct genomic positions. Note that a Naïve Bayes network and formula depiction is used to illustrate the concept of a suitable computational model, preferably in the form of a linear classifier with independent feature sampling in the context of present invention. Importantly, the present invention is not limited in its scope to the Naïve Bayes algorithm but encompasses and utilizes as in the presented examples any linear classifier with independent feature sampling. Suitable algorithms may be also but not limited to other generalized additive models including artificial neural networks functioning as linear classifier with independent feature sampling.

Alternative suitable computational models with independent feature sampling in the context of the present invention, include, but are not limited to, logistic regression models, support vector machines and generalized linear models.

To further illustrate the present invention, FIG. 1c depicts a network with a larger number of input nodes, which may be any number of genetic and/or epigenetic information from any number of genomic positions. The black and white colouring of the input nodes represent binary information from said input nodes from distinct patient derived samples. These subsamples exemplify random subsets in the context of the present invention. Based on all the input nodes, a disease class probability can be computed by the computational model. In FIG. 1d the input nodes have been divided into two subsamples. Based on the function of said computational model described in the present invention, both resulting disease class probabilities will be with some statistical variation, equivalent. FIG. 1d depicts two intentionally chosen subsets of genetic and/or input information. Such an intentional subsetting of existing data is referred to in the literature as bootstrapping. Such intentional subsetting or bootstrapping can be used to test the quality of a computational model from existing datasets as outlined in Example 54 in the present invention. Similarly, genetic and/or epigenetic information from distinct genomic positions can be stratified based on additional information pertaining to the genomic positions. For example, genetic and/or epigenetic information can be selected based on if this information is located on a chromosome arm with significant copy number variability in a tumor class or all those genomic positions could be subsetted, which belong to a gene promotor. This stratified sampling strategy can also be used evaluate the performance of sample class specific computational models.

Others have used bootstrap and stratified sampling strategies to evaluate competing methods. Kuschel et al 2021 have used bootstrapping to demonstrate the robustness of their competing Random Forest classifier with ad hoc training with exiting datasets. Importantly, Random Forrest classifiers cannot treat input genetic and/or epigenetic information from a single distinct genomic region independently from other single distinct genomic regions, as Random Forrest classifiers necessarily link two or more such information with each other in so called decision trees. This is particularly illustrated in the study of Capper et al. 2018 in the “Extended Data FIG. 9 Development of the random forest classifier” panel b. Therefore, competing methods such as described by Capper et al 2018, Kuschel et al 2021 or Bie et al 2022 cannot classify samples on line from a stochastic source as their computational models invariably rely on models which evaluate genetic and/or epigenetic information from one position always in the context of genetic and/or epigenetic information predetermined by said Random Forest model. As the Random Forest model cannot anticipate the stochastically selected genetic and/or epigenetic information provided by a nanopore sequencer, the Random Forest classifier will always have to wait until a by the computational model predefined set of linked information from a predefined set of genomic positions has been sequenced or it is necessary to retrain the classifier on the random subset of genomic positions, which have been obtained by the stochastic nanopore sequencing up to a timepoint in the sequencing process.

In contrast to said competing methods the present invention allows to compute online sample class probabilities from a random subset of genetic and/or epigenetic information from a stochastic source such as a nanopore sequencer sequencing randomly selected genomic positions from a DNA sample without having to wait until a predefined set of genomic positions has been sequenced nor has the computational model of the present invention to be retrained for obtaining reliable sample class assignments after a random subset of genetic and/or epigenetic information has been obtained, as it is necessary with competing methods (Kuschel et al. 2021).

A “stochastic source” in the context of the present invention is a set of genetic and/or epigenetic information from a random subset of distinct genomic positions of a given genome or multiple genomes present in a given sample. Such random subsets are a frequent phenomenon in genomic analysis applications as in most next-generation sequencing molecular workflows large genomes at the size of mega- to gigabases are broken into smaller fragments with the size of tens of to thousands of bases and sequenced in random order by sequencing apparatus. Such sequencing workflows have also been frequently referred to as “shotgun sequencing” due to their stochastic nature. Notably, nanopore sequencers provide the genetic and/or epigenetic information in a time resolved sequential manner, as a smaller number of nanopores sequence tens to thousands of fragments in parallel from a pool of millions to billions of said DNA fragments over the time course of minutes to days.

Said sampling process from a stochastic source at a time point, where a number of genomic positions have been sequenced by a nanopore sequencer is depicted in FIG. 1e where said at random selected subset of genomic positions is provided to the computational model. Such a random subset in the context of the present invention of genetic and/or epigenetic information from a random set of genomic positions cannot be considered bootstrapping nor stratified sampling. FIG. 1f depicts a second random subset of genetic and/or epigenetic information from a subset of distinct genomic positions from the same sample as in FIG. 1e from a stochastic source e.g., a nanopore sequencer at another timepoint and its application to the same computational model as in FIG. 1e in the form of a linear classifier with independent feature sampling as outlined in a preferred embodiment of the present invention. The equal sign in between FIG. 1e and FIG. 1f indicates that even though both subsets were selected at random from a stochastic source the application of said computational models based on the present invention will lead within reasonable statistical bounds to equivalent sample class probability results.

Said computationally challenging sampling from a stochastic source can, in a non-limiting example, be accommodated by the present invention by employing assumptions relating to Poisson and/or Bernoulli sampling in the training of the computational model as well as using sample class specific weighting matrices towards satisfying the equivalence of results from any random subset of epigenetic and/or genetic information from distinct genomic positions as illustrated in FIG. 1d, f and e.

In the context of the present invention, each genetic and/or epigenetic information from a genomic position is processed independently from any other genetic and/or epigenetic information from another genomic position, because the stochastic source provides such information in a time resolved manner and thus no interaction or conditional evaluation of said information from different genomic locations can be performed. This is visualized in the network depictions of the computational model where all nodes are treated independently and have no lines between them. Such independently evaluated genetic and/or epigenetic information is then summarized in a class log-likelihood. Also in this summarization step, no conditional interaction of said information in the computational model is performed, unlike in competing methods such as Random Forest decision trees.

Note that a Naïve Bayes computational model for example computes from the aforementioned summarized log-likelihoods class probability estimates. For simplicity and explanatory reasons, only the transformation and summarisation of independently sampled genetic and/or epigenetic information from genomic positions into summarized log-likelihoods is discussed with the subsequent steps leading to a disease class probability estimate implied.

To exemplify the procedure, consider a CpG that has been determined to be methylated by a nanopore sequencer within milliseconds after a DNA molecule has passed through a nanopore. The said CpG is generally found to be methylated in cancer class A but not in B and this is also reflected in the trained computational model.

In a most simple, non-functional but explanatory and hypothetical computational model according to the present invention for said CpG in cancer class A, the comparison of the value “1” for a methylated CpG at said position with the expected model value “1” for methylated is transformed into a log-likelihood value of “−0.1”. In a most simple hypothetical computational model for said CpG in cancer class B, the comparison of the methylation value “1” for a methylated CpG at said position with the expected value “O” in said cancer class for an unmethylated CpG is transformed into a log-likelihood value of “−0.2”. Therefore, the summarized log-likelihood after the independent evaluation of said CpG is “−0.1” for cancer class A and “−0.2” for cancer class B.

Next, a second CpG is sequenced by the nanopore sequencer from another genomic position and as in the previous case, the CpG is determined to be methylated in the cancer sample. Also again, the trained computational model expects the CpG to be methylated in cancer class A but not in B. Consequently, the log-likelihood value “−0.1” is added to the cancer class log-likelihood A and the log-likelihood value “−0.2” is added to the cancer class log-likelihood B. After the second CpG has been sequenced, the summarized cancer class log-likelihood for cancer class A is “−0.2” and the summarized log-likelihood for cancer class B is “−0.4”.

Finally, a third CpG is sequenced by the nanopore sequencer from another genomic position and again as in the previous case, the CpG is determined to be methylated in the cancer sample. Also again, the trained computational model expects the CpG to be methylated in cancer class A but not in B. Consequently, the log-likelihood value “−0.1” is added to the cancer class log-likelihood A and the log-likelihood value “−0.2” is added to the cancer class log-likelihood B. After the third CpG has been sequenced, the summarized cancer class log-likelihood for cancer class A is “−0.3” and the summarized cancer class log-likelihood B is “−0.6”.

This most simple explanatory example shows that before the summarisation of independently derived class log-likelihoods from each independently measured genetic and/or epigenetic information, no interaction term derived from the information from the first and second CpG is computed or performed in the computational model in the present invention in contrast to e.g. Random Forest Decision trees.

This most simple example demonstrates only the steps in the computational model but as it is clear to a person knowledgeable in the art, each step described can contain any number of transformations and mathematical operations on the obtained genetic and/or epigenetic information from a genomic position. Due to the stochastic nature of the sequencing process, such transformations and mathematical operations can be performed on each genetic and/or epigenetic information independently from all other genetic and/or epigenetic information and the resulting log-likelihood values obtained from a number of independently evaluated genetic and/or epigenetic information are summarized to a disease class log-likelihood with any number of transformations and mathematical operations.

Note that in the context of the present invention, the order at which the independently obtained CpGs are sequenced is not used by the computational model due to the stochastic nature of the sequencing process. Therefore, the summarisation of the independently computed log-likelihoods from each of the independently sampled CpGs will have to consider each information from each CpG independently from all other information from other CpGs in the summarisation step.

In this most simple example, the summarisation step consists of adding up all independently computed log-likelihood values from each CpG. This summarization step will approximate the sample class log-likelihood irrespective of the order on how the information was obtained from each CpG and irrespective of what CpG from a large number of possible CpG was obtained. Consequently, the probability estimate will be independent if only the first or the second or the third CpG was sequenced or only information from the first and the second or the first and the third or the second and the third or the first, second and third was obtained.

In some preferred embodiments of the present invention, to compute comparable results between sample class log-likelihood estimates from only the first CpG or the second CpG or the third CpG or from the first CpG and the second CpG or the first CpG and the third CpG or the second CpG and the third CpG or the first, second and third CpG it, can be desirable to perform a standardization of the summarised sample class log-likelihood estimates. In a most simple way, the sample class log-likelihoods can be computed by simply dividing the summarized sample class log-likelihoods from each of the listed combinations by the number of CpGs included in each of the listed CpG combinations.

In the context of the present invention the term “standardization” refers to any computational steps intended to transform summarisation results from the computational model from independently sampled subsets of genetic and/or epigenetic information from genomic positions in order to allow for comparable results between results obtained from independent random subsets of genetic and/or epigenetic information.

This most simple example demonstrates only the summarisation and standardization step in the computational model but as it is clear to a person knowledgeable in the art, either step described can contain any number of transformations and mathematical operations on the obtained genetic and/or epigenetic information from genomic positions.

In accordance with a preferred embodiment of the present invention, the data from said sample in step a) is obtained by: i) isolating genomic DNA from said sample, ii) preparing a DNA library by fragmentation of said isolated genomic DNA; iii) sequencing of the DNA fragments obtained in step ii), thereby determining the sequence of nucleotides of said DNA fragments, iv) comparing the sequence of nucleotides of each individual DNA fragment with the sequence of nucleotides of a reference genome, and v) determining the genetic and/or epigenetic information of a genomic position from each DNA fragment in comparison to said reference genome, thereby providing data comprising genetic and/or epigenetic information of a random subset of genomic positions.

In accordance with a particularly preferred embodiment, steps i) to v) of the preferred embodiment described above are completed within less than 6 hours, preferably within less than 2 hours, more preferably within less than 1 hour and most preferably within less than 45 minutes.

Genomic DNA may be isolated from the sample by any suitable means and methods available from the art (Green and Sambrook, 2012), including commercially available kits. As described in the examples herein below, genomic DNA may be isolated from a tissue sample using e.g. the QIAamp Fast DNA Tissue Kit (QIAGEN, Hilden, Germany). The kit may be used according to the manufacturer's instructions. Alternatively, the kit may be used as described in Example 1 herein below to reduce the sample processing time and handling steps while maintaining sufficient sample purity for further preparation of a DNA library.

The DNA library may be prepared by any suitable means and methods available from the art (as for example described in Green and Sambrook, 2012), including commercially available kits. Typically, the steps for preparing a library from isolated genomic DNA include fragmentation of the isolated genomic DNA, attachment of oligonucleotide adaptors to the ends of the DNA fragments, amplification the adapter-fragment complexes, and quantification of the final library product.

In accordance with a particularly preferred embodiment, the DNA library in step ii) is obtained by fragmentation of said genomic DNA of step i) by a transposase, the addition of tagging adapters to the DNA fragments cleaved by said transposase and the subsequent attachment of sequencing adapters to said tagging adapters, with said sequencing adapters carrying a motor enzyme with helicase functionality suitable for the initiation of nanopore sequencing of said transposase tagged genomic DNA.

Such sequencing adaptors are, for example, provided by Oxford Nanopore Technologies sequencing kits such as the SQK-RAD004 Rapid Sequencing Kit.

The term “transposase” as used herein refers, e.g. to a MuA transposome complex consisting of double stranded DNA molecules bound to four MuA proteins forming the tetrameric MuA transposome. Said transposase can act as an DNA processing enzyme by fragmenting a double-stranded DNA-fragment into two DNA fragments and can attach double stranded DNA molecules (referred to herein as “tagging adapters”) bound to the MuA transposome complex to said DNA fragments. Said double stranded DNA molecules (i.e. tagging adapters”) are then attached by the transposase to the DNA fragment and can then be utilized as “mark” or “tag” for subsequent attachment of other nucleotide sequences.

The term “tagging adapter” as used herein generally relates to a polynucleotide comprising a double stranded DNA molecule bound to said MuA transposome complex, which can be attached by said transposase to DNA fragments cleaved by said transposase. The tagging adapter can be generated synthetically and is constructed to allow for rapid and specific attachment of other adaptors to the other end of the tagging adapter.

The term “motor enzyme with helicase functionality” as used herein refers to a class of enzymes that move directionally along a nucleic acid backbone, separating two annealed nucleic acid strands, i.e. a DNA, RNA or RNA-DNA hybrid, using energy derived from ATP hydrolysis or other sources. Motor enzymes include, but are not limited to, helicases, such as HEL 308 helicases, genetically modified versions of the HEL308 helicase or other molecular motors. A Hel308 helicase can control the movement of a polynucleotide through a nanopore especially when a potential, such as a voltage, is applied. The helicase is capable of moving a target polynucleotide in a controlled and stepwise fashion against or with the field resulting from the applied voltage. The helicase is capable of functioning at a high salt concentration which is advantageous for characterizing the polynucleotide and, in particular, for determining its sequence using strand sequencing.

The term “nanopore sequencing” as used herein refers to a sequencing technique that enables direct, real-time analysis of nucleic acid fragments by monitoring changes to an electrical current as the nucleic acid fragment is passed through a nanopore, wherein the resulting signal provides information on the specific sequence of nucleotides of said nucleic acid fragment.

The term “nanopore” as used herein generally refers to a pore, channel or passage formed or otherwise provided in a membrane.

The term “sequencing adapters carrying a motor enzyme with helicase functionality suitable for the initiation of nanopore sequencing” refers to sequencing adaptors that initiate the attachment of a DNA fragment to a nanopore in a nanopore sequencing flow cell, such as the MinION FLO-MIN106D Flow Cells. Initiation includes unwinding the DNA double helix of said DNA fragment and guiding a single DNA strand from said DNA fragment through said nanopore and slowing down the movement of the single DNA strand moving through the nanopore, thereby allowing measurements of an ionic current across the nanopore which changes characteristically with the sequence of nucleotides of said single DNA strand.

As described in the examples herein below, the DNA library may be prepared using e.g. the SQK-RAD004 Rapid Sequencing Kit (ONT, Oxford, United Kingdom). The kit may be used according to the manufacturer's instructions. Alternatively, the kit may be used as described in Example 1 herein below to increase the sequencing throughput during nanopore sequencing.

In the context of brain tumor classification as described in the examples herein below, it was found that the preparation of the sequencing library benefits from increase DNA input ranging from 600 to 700 ng. However, also amounts of DNA lower than 600 ng may be used.

Thus, in accordance with a particularly preferred embodiment, the amount of isolated genomic DNA for the preparation of the DNA library in step ii) is preferably above 25 ng, more preferably above 50 ng, more preferably above 100 ng, more preferably above 200 ng, more preferably above 400 ng, more preferably above 600 ng and most preferably above 700 ng.

Sequencing of the DNA fragments in step iii) may be performed by any suitable means and methods known in the art. These include, but are not limited to, whole genome bisulfite sequencing, such as sequencing bisulfite treated DNA and control DNA with Illumina short read sequencing (Lister et al., 2009), targeted bisulfite DNA sequencing, such as Illumina short read sequencing libraries prepared with the TruSeq Methyl Capture EPIC Library Prep Kit (Illumina) and nanopore sequencing.

In accordance with a particularly preferred embodiment of the invention, nanopore sequencing is used in step iii).

The comparison of the sequence of nucleotides of each individual DNA fragment with the sequence of nucleotides of a reference genome in step iv) of the preferred embodiment described above is also often referred to as “alignment”. Depending on the read out, step v) of the preferred embodiment described above may comprise determining either a) a deviation in the sequence of nucleotides with respect to the reference genome, b) the presence or absence of an epigenetic modification (e.g. cytosine od adenosine methylation) with respect to the reference genome or c) local enrichment of sequencing reads (in the case of histone modification measured by CHIP-seq or open-/closed chromatin measured with ATAC-seq or cfDNA).

For steps iv) and v) of the preferred embodiment described above a reference genome that is a digital representation of the DNA sequence from an idealized individual organism of the human species may be used, such as for example the GRCH38.p13 reference genome. Alternatively, a reference genome from a single, nearly completely homozygous and immortalized cell line, as for example the CHM13 T2T v1.1 reference genome may be used. In accordance with the preferred embodiment described above, any symbolic representation of a human genome can be utilized as reference genome as long as it allows the matching of sequenced DNA fragments with one or multiple positions in its coordinate system and the comparison of the sequenced DNA information (i.e. genetic and/or epigenetic information) with the reference genome sequence representation.

Epigenetic modifications such as histone modifications can be read out by short read and nanopore sequencing by sequencing DNA fragments obtained by Chromatin Immuno Precipitation sequencing (CHIP-seq) or related methods such as CUT & RUN sequencing. This DNA sequence information can be compared to the sequence of a reference genome and provides information about epigenetic marks on proteins attached to DNA or the location of proteins at specific DNA positions. Similarly, epigenetic states and histone positions can be read out by short read sequencing methods with an Assay for Transposase-Accessible-Chromatin using sequencing (ATAC-seq) and related methods.

The term “epigenetic state” as used herein refers to distinct states of epigenetic information encoded on the genomic sequence. In its simplest form, a CpG at a specific genomic position can be either methylated or not methylated. In this context, a methylated CpG at a specific genomic position is one epigenetic state and a not methylated CpG at the same specific genomic position is another epigenetic state of said CpG. Similarly, other epigenetic marks such as histone methylation or acetylation, open or closed chromatin can be read out by sequencing and processed by the present invention as epigenetic states. Similarly, the term “genetic state” as used herein refers to distinct states of genetic information encoded on the genomic sequence. Accordingly, the genetic and/or epigenetic information may comprise information about such genetic and/or epigenetic states.

In accordance with a preferred embodiment of the present invention, the genetic and/or epigenetic information of the random subset of genomic positions of said sample is binary or alternatively non-binary.

In this respect it is important to note that real-time assessment of sequencing data from, e.g. nanopore sequencing requires the handling of sparse, shallow coverage data.

The term “coverage” or “coverage data” as used herein refers to how many times a genome has been sequenced. A human reference genome consists of approximately 3 billon bases. A 100% or 1-fold coverage of a human genome refers to 3 billion bases that have been sequenced from a human sample. As the sampling process in e.g. a nanopore sequencer can be viewed as a Poisson sampling process, at 1-fold coverage, some genomic positions will have been sampled several times, some genomic positions will not have been sampled at all, yet most genomic positions will have been sampled once.

For example, a distinct genomic position is covered once, 2-fold, 3-fold, 4-fold etc. if 6 billion, 9 billion, 12 billion etc. bases from a human genome has been sequenced.

It is important to note that during nanopore sequencing only a few genomic positions can be sequenced in a short time by a nanopore sequencer. A MinION nanopore sequencer can sequence and analyse the genomic information about up to 50 million bases within five minutes. At other instances a MinION nanopore sequencer can sequence and analyse the genomic information about up to 160 million bases within 30 minutes It is obvious that at this rate, sample specific information pertains to (“covers”) only less than 0.2% of the human genome after five minutes and in another instance covers only less than 0.6% of the human genome after 30 minutes. At this coverage, nearly all the sequenced positions in a reference genome are covered only once.

Accordingly, in a particularly preferred embodiment, an individual genomic position is sequenced 1-fold, preferably 2-fold, more preferably 3-fold and most preferably 4-fold during sequencing in step iii) of the preferred embodiment described above.

The term “sparse coverage” or “low coverage” as used herein refers to the situation, where only a certain percentage of a genome, e.g. human genome with approximately 3 billion bases has been sequenced, so e.g. a small percentage as low as 0.2% (50 million bases, 0.02 fold coverage) up to less than 100% (3 billion bases, 1 fold coverage) up to 1000% (30 billion bases, 10 fold coverage).

The term “shallow coverage” as used herein refers to the fact, that with 100% coverage most of the human genome will be covered only once, thus providing for most genomic positions only one information. 100% coverage is referred to as 1-fold coverage.

In contrast the term “deep coverage” in the context of the present invention refers to sequencing of the complete human genome multiple times from a single sample, such as more than 40 times the DNA sequence of a human genome, resulting in a coverage of about 120 billion bases. This coverage is referred to as 40-fold coverage.

The term “binary” in accordance with the preferred embodiment described above refers to the case where a genomic position is covered only once and the genetic and/or epigenetic state of an individual genomic position is determined only once. For any CpG at a specific genomic position in the case of such shallow, sparse coverage, the methylation of said CpG can only be determined to be either methylated or not methylated. As only two states, methylated or not methylated can be determined, the resulting information or read out epigenetic state is necessarily binary.

The sparse, shallow coverage data results in e.g. a binary read out of epigenetic states of methylated and not methylated CpGs at specific genomic positions. As outlined above and illustrated in FIG. 1, the sparse, shallow coverage data is also a random subset of specific genomic positions if a sequencing process is the source of said data. The described exemplary type of binary data is effectively approximated with a Bernoulli sampling process from a stochastic source.

As illustrating example, a nanopore sequencing library from step ii) of the preferred embodiment described above may contain a very large number (i.e. many billions) of DNA fragments from a human genome derived from a tissue sample. During nanopore sequencing of this library in step iii) each of the up 512 active nanopores in a MiniION flow cell sequences DNA fragments from said library at random, discards the fragment and continues within only few seconds by sequencing another DNA fragment from said library also in random order. Since the size of the DNA fragment library from step ii) is in this example several orders of magnitude larger compared to the number of fragments sequenced by the nanopores, this process can be modeled as a “sampling process with replacement”.

The term “sampling process with replacement” refers to sampling process wherein a sampling unit is drawn from a finite population and is returned to that population (i.e. DNA fragments in a sequencing library from step ii), after its characteristic(s) have been recorded, before the next unit is drawn.

As a result, the measurements by the nanopore can be modeled as independent measurements and the number of times a specific DNA fragment and its genetic and/or epigenetic state is observed can be approximated by a Poisson distribution. In the case of sparse, shallow coverage data, the majority of genetic and/or epigenetic information of an individual genomic position in the random subset of genomic positions is not observed multiple times. In accordance with the present invention, in this situation the sampling process can be approximated as a Bernoulli sampling process, where all of genetic and/or epigenetic information is observed only once with equal probability.

It is important to note that any subset of regardless of either Poisson or Bernoulli sampling methodologies is applied to said subset of position-specific genetic and/or epigenetic information obtained through nanopore sequencing can be converted into a Bernoulli or Poisson sample by removing sampled genomic positions from said subset, as long as the majority of greater than 50% of the genetic and/or epigenetic information can be kept for processing by the computational model. Such cases include but are not limited to multiple genetic and/or epigenetic information sampled for the same genomic position and/or genetic and/or epigenetic information sampled from the same DNA fragment as said multiple information derived from a single DNA fragment cannot obey a requirement of complete independence between each information processed by the computational model. In this specific case, stratified sampling methodologies may be applied such as selecting a single CpG from a single read, even though multiple CpGs with associated epigenetic information are present on the same nanopore read. Note that this subsampling is utilized in order to ensure the complete independence requirement of e.g., a Bernoulli Naïve Bayes classifier computational model, but that this subsampling step is only applied after the data in form of a sequencing read is obtained from a stochastic source, which can never be a stratified sampling process due to the stochastic nature of the shot gun sequencing process.

As shown in the examples herein below, such binary genetic and/or epigenetic information may be e.g. obtained from nanopore sequencing. In the context of brain tumor classification as described in the examples herein below, the genetic and/or epigenetic information of the sample sequences by nanopore sequencing thus comprises information about an individual methylation event for each genomic position within the random subset of genomic positions rather than average methylation rates, as e.g. provided by other sequencing techniques.

In the context of the present invention, the term “methylation event” relates to a binary methylation event at a specific position in a given DNA strand from a given DNA sample, as e.g. detected by a methylation aware basecaller such as Nanopolish or Oxford Nanopore Technologies' (Guppy, Megalodon or Bonito) or related methods from nanopore data.

The term “non-binary” as used herein refers to the case, where multiple categorical genetic and/or epigenetic states of a genomic position are determined and/or the genetic and/or epigenetic states of a genomic position are represented by a measured variable that is non-binary e.g. multiple categorical states and or encoding of those categorical states as β-values between 0 and 1, or any other real number.

In accordance with another preferred embodiment of the present invention, the genetic and/or epigenetic information of the set of genomic positions is binary or alternatively non-binary.

The term “set of genomic positions” as used herein refers to all genomic positions for which genetic and/or epigenetic information is available for training a computational model.

As an illustrating example, a human genome consists of 3 billion bases. In the current reference genome GRCH38.p13 there are approximately 28 million CpGs which can be methylated or unmethylated. Whole genome nanopore sequencing can determine the methylation status from all of the 28 million CpGs. Other methods for the determination of CpG methylation status determine CpG methylation rates as β-values from a subset of those 28 million CpGs. Those sets of CpGs are considered “genome-wide” as those CpGs were not selected based on any forms of a sample class-specific feature set. The selection of those genome-wide CpG features has been historically based on technological limitations, such as limited number of CpG-specific probes that can be placed on a microarray or cost considerations such as that whole genome bisulfite sequencing for the determination of all CpGs in a human genome has been prohibitively costly for diagnostic applications.

For example for such technologically limited genomic position sets, the Illumina TruSeq EPIC method determines the methylation rate for approximately 3.3 million CpGs by enriching for genomic fragment with hybridization capture probes and subsequent bisulfite sequencing, the Illumina Infinium EPIC BeadArray determines the methylation rate for approximately 800 thousand CpGs with hybridization probes on a microarray, and the Illumina Infinium 450k BeadArray determines the methylation rate for approximately 450 thousand CpGs with hybridization probes on a microarray.

As the most comprehensive training dataset currently available encompassing 2800 samples from 91 sample classes was generated with Illumina Infinium 450k BeadArrays (Capper et al. 2018), the set of genomic positions for training based on this dataset would be limited to said 450,000 genome wide positions. Given a dataset providing measurements of the methylation status of all 28 million CpGs from samples with predetermined sample classes, the computational model of the present invention can be trained on all 28 million genomic positions and can determine a sample class assignment based on a randomly selected subset of those 28 million genomic positions.

For example, in case a SNP is present at a specific genomic position, all four bases (Adenine, Cytosine, Guanin, Thymidine) could be present and measured at this position. As there are four different possible genetic states, these categorical variables (A, T, G, C) must be considered non-binary.

In another instance a structural variation such as a tandem repeat expansion may be present at a distinct genomic location with 2, 3, 4, 5, 6, . . . etc. repeats at this position. As there are numerous different possible genetic states, these natural numbers encoding the tandem repeat number must be considered non-binary.

In another instance a CpG may be present at a distinct genomic location and information on the CpG methylation has been obtained with deep coverage or with Illumina DNA Methylation arrays. In such a case, the CpG methylation is measured and reported as methylation rate, i.e. a β-value between 0 (representing no methylation of said CpG) and 1 (representing full methylation of said CpG) with a continuous variable between 0 and 1 (e.g. 0.1; 0.2; 0.3; 0.4; . . . 0.8; 0.9; or 1). As there are numerous different possible methylation rate values, these must be considered non-binary.

In another instance a CpG may be not methylated, methylated or hydroxymethylated. Each of these categorical values can be determined e.g. by deep coverage nanopore sequencing as β-values. For hypothetical a read out could look like [CpG-methylated ß-value=0.25] [CpG-hydroxymethylated ß-value=0.25]. This expression describes a CpG which is found to be methylated in 25% of all measurements and found to be hydroxymethylated in 25% of all measurements and found to be not methylated in 50% of all measurements. This combination of categorical values and the specifying ß-values is referred to herein as “non-binary”.

In another instance open and closed chromatin states have been measured at a distinct genomic position and information on the epigenetic state has been obtained by sequencing DNA fragments originating at the open chromatin at said distinct genomic position with ATAC-seq. Here, many reads originating at said position represent open chromatin, no sequencing reads originating at said genomic position represent closed chromatin. As the open and closed chromatin states are represented by sequencing reads, a continuous variable from 0 reads to 10, 20, 40, 50, 60, . . . etc. reads may represent the epigenetic open or closed chromatin state. As there are numerous reads possible to originate from said genomic position, these must be considered non-binary. Accordingly, in sequences obtained from cfDNA samples, open or closed chromatin can be detected by mapping cfDNA fragment start positions, the respective fragment length in an analogue manner.

In another instance a genomic position in a genome might be deleted in one or both alleles of a diploid mammalian genome. Such genomic CNVs (copy number variations) can be detected by aligning sequencing reads to a reference genome and counting the number of sequencing reads in predefined regions of the reference genome. Such a predefined region of a reference genome is often also referred to as a “bin”. Said bin can encompass any continuous region of a reference genome. One example is the long arm of chromosome 1 of the human genome encompassing approximately 120 Megabases of the reference genome or only the approximately 50 Kilobases of the CDKN2A/B gene. In such a case, reads aligned to a genomic bin may be counted and the value compared with other genomic regions, which are not amplified or deleted. In such a case, deletion of one or both alleles contained in the bin will result in less read counts and amplification of said genomic region will lead to an increased read count relative to genomic regions which are not amplified or deleted. Several statistical methods have been proposed to infer from such bin-related read counts the exact copy number (Magi et al, 2019; Xie and Tammi, 2009). For illustrative purposes, it is assumed that a not-amplified and not deleted bin relative to another not altered genomic bin will be represented by values close to 0 and a deleted allele will result in relative bin count values close to −1 and both deleted alleles will result in relative bin count values close to −2. Similarly, amplification of one allele will result in relative bin count values close to +1, amplification of both alleles will result in relative bin count values close to +2. These CNV associated, genomic position specific bin count values are also referred to herein as “non-binary”.

Training of the computational model in accordance with step b) of the method of the invention may comprise a step of determining class separability (FIG. 5).

Towards this aim, the user of the computational model used in the method of the invention wishes to determine if the computational model performs satisfactorily for assigning a sample to a sample class with sufficient accuracy and reliability. Accordingly, the step of data preparation may include the determination of class separability. The term “class separability” as used herein refers to how successful distinct tumor classes can be separated based on data derived from representative samples from those distinct tumor classes. Good class separability can be assumed if only a few or ideally no samples from one class are misclassified as belonging to another tumor class based on a data set derived from said samples. Unsatisfactory class separability can be assumed if many samples from one class are misclassified as belonging to another tumor class on a data set derived from said samples. No class separability can be assumed if all samples from one or more sample classes are misclassified as belonging to another sample class on a data set derived from said samples.

In accordance with the present invention, class separability can be estimated based on employing existing dimension reduction algorithms or supervised classification methods as outlined below. This first step of determination of class separability represents a good practice approach towards establishing reliable results of the method of the invention but is ultimately not necessary.

Accordingly, in a particularly preferred embodiment of the invention, training of the computational model includes the determination of class separability.

The separability of an a priori defined sample class (e.g. a specific tumor class, such as IDH-mutant oligodendroglioma) from other sample classes may be estimated based on global unsupervised or supervised dimension reduction techniques such as Principal Component Analysis (PCA), Uniform Manifold Approximation and Projection (UMAP) classification, Partitioning Around Medoids (PAM), Mixture Discriminant Analysis (MDA), Least Absolute Shrinkage and Selection Operator (LASSO) and Gaussian Finite Mixture Models (as provided by the Mclust R package). Such supervised or unsupervised dimension reduction and/or visualization techniques are used to identify sample classes by their respective a priori defined and known class labels which then can also be easily separated by the computational model; see also FIG. 11.

The term “class label” as used herein refers to a diagnostic category associated with a specific sample from a subject with a distinct disorder. Such class label can be derived from other means of multimodal diagnostic characterisation of a disorder, such as, but not limited to, medical history, clinical findings, blood tests, histology, determination of molecular markers or the longitudinal observation of patients suffering from a distinct disorder and recording of clinical outcomes such as prognosis, response to treatment or survival rates. In the case of brain tumors, class labels are assigned following the guidelines of the 2021 WHO Classification of Brain Tumors (Louis et al, 2021)). It is important to note that the term “class label” in the context of the present invention can also refer to subgroups of a distinct disorder without clinical or diagnostic definition, which is, however, currently widely accepted by experts in the field. In specific cases, such distinct class labels can be discerned e.g. based on genome wide DNA methylation patterns, without a known clinical distinction of those separable subtypes. In such a case, class labels such as “Subtype A” and “Subtype B” may be used as class labels.

It is understood that in a preferred embodiment of the present invention, class labels are chosen in relationship to medical diagnosis in such a manner that a person knowledgeable in the art of medicine can use said class label after it has been evaluated by the computational model as described in the present invention for a patient derived sample, as medical diagnosis.

Training of the computational model in accordance with step b) of the method of the invention may further comprise a step of optimization of class separating parameters (FIG. 5).

In this step it is determined if one sample class is best modelled in the training procedure with one or more centroids representing groups of samples from one sample class.

The term “centroid” as used herein generally relates to the mean position of all of the points in all of the coordinate directions of any object in n-dimensional space. In accordance with the present invention, of a sample class consisting of pre-classified samples may e.g. be the center of β-values from a random subset or all of the CpGs on an Illumina Infinium 450k BeadArray from a single pre-classified sample or a group of pre-classified samples comprised of samples from a sample class. In accordance with the present invention, a centroid can also be represented by the medoid from the same group of samples or even any combination of values to define a prototypical value from the mentioned data source capable for classifying samples based on the computational model. A “medoid” as used herein is a single sample selected from a sample class consisting of pre-classified samples that has been determined to be the most representative sample for said sample class by any metric or computational model known to those knowledgeable in the art. Example 3 of the present invention describes the use centroids for the successful training of computational models as described in the present invention.

This step of determination of centroids or medoids also represents a good practice approach towards establishing reliable results of the method of the invention but is ultimately not necessary, as any linear model with independent feature sampling can be applied in the context of the present invention, even in the case if centroids or medoids are non-informative for distinguishing class labels.

Also, at this step, the separability of two distinct biologically defined sample classes can be determined.

Training of the computational model in accordance with step b) of the method of the invention may further comprise a step of consideration of batch effects (FIG. 5).

In case the sample class models are trained on non-binary genetic and/or epigenetic information (e.g. methylation rates from array-based and high content CpG methylation sequencing methods) but the class membership probability is determined on binary genetic and/or epigenetic information (e.g. single methylation events obtained from e.g., nanopore sequencing), both datatypes need to be adjusted with a normalization procedure. Any suitable normalization procedure known in the art which removes so-called batch effects can be applied.

In molecular biology, a batch effect occurs when non-biological factors in an experiment can cause changes in the data produced by the experiment. Such effects can lead to inaccurate conclusions when their causes are correlated with one or more outcomes of interest in an experiment.

In the context of the present invention, the determination of genetic and/or epigenetic information (e.g. methylation rates) with e.g. microarrays or bisulfite sequencing-based methods and nanopore sequencing represents such a systematic, non-biological factor. Similarly, building a computational model based on methylation rates (from any of the methods suitable for determining CpG methylation rates for any given CpG for a sample class) and applying the model to binary methylation events from a nanopore sequencing run from a sample to be evaluated by the computational model, has to be considered to be potentially caused by batch effects.

Accordingly, this step of consideration of batch effects is used to estimate the error rate per overall genomic positions (e.g. CpGs) and for each genomic position included in the model. In the context of CpG methylation, for example, at least three (or more) matching samples with CpG methylation rates determined on the training and nanopore platform can be compared as two batches with matching samples. The error rate is then determined for each CpG and the resulting CpG-specific value is used in conjunction with the methylation call produced e.g. by ONT Megalodon of the CpG methylation event determined by nanopore sequencing; see also FIG. 12.

This step of batch correction represents a good practice approach towards establishing reliable results of the method of the invention but is ultimately not necessary.

Training of the computational model in accordance with step b) of the method of the invention comprises a step of relating event rates with single events (FIG. 5).

Thus, in accordance with a preferred embodiment of the invention, the computational model is trained by optimization of the classification accuracy using a statistical model which relates the probability of observing binary or non-binary genetic and/or epigenetic information in a random subset of genomic positions to a set of pre-determined genetic and/or epigenetic information in the form of event rates obtained from a plurality of pre-classified samples of known diseases.

For training a computational model suitable for the real-time analysis of a stream of genetic and/or epigenetic information derived from nanopore sequencing such as a Naïve Bayes classifier, event rates in sample classes of pre-classified samples need to be linked with single events from a single sample.

The term “event rate” as used herein relates to rates of observed genetic and/or epigenetic states empirically observed for a genomic position. The most obvious example for event rates in the context of the present invention are methylation rates encoded as ß-values measured by Illumina Infinium 450k arrays.

The term “event” as used herein refers to the single measurement of a genetic and/or epigenetic state at a genomic position in a single sample subjected to the predictive computational model described herein. The most obvious example for an event in the context of the present invention represents also the most extreme case for such an observed event: Nanopore sequencing can output methylation calls from single molecules of DNA from a sample to be analysed by the computational model outlined according to the present invention.

In accordance with the preferred embodiment described above, the computational model employs multinomial distributions to link single events to event rates.

In the context of the present invention, it is understood that utilization of multinomial distribution assumption is part of the training of said computational models in the form of linear classifiers with independent feature sampling.

The term “multinomial distribution” is used herein as a general term for use of several distributions known in the art for different technologies and/or measurements of genetic and/or epigenetic states at a genomic position. These subgroups of multinomial distributions include, but are not limited to Bernoulli distribution, Poisson distribution, Categorical distribution, binomial distribution, beta-binomial distribution and products thereof.

In the context of the present invention, multinomial distributions as mentioned above enable the computationally efficient and effective training of said computational models on a large or even nearly unlimited number of independent genetic and/or epigenetic features in parallel. Also, the utilization of said multinomial distributions allow for the computationally efficient and effective optimization of computational models in the form of a linear classifier with independent feature sampling on a large or even theoretically unlimited number of independent genetic and/or epigenetic features in parallel.

The term “optimization” in the context of the present invention relates to, but is not limited to, improving the sensitivity and/or specificity of said computational model for specific sample classes or diagnoses. Optimization can be performed by defining a cost function and retraining the initial computational model to minimize said cost function. As an example, in the case of a Naïve Bayes classifier, the misclassification error between two sample classes can be used as cost function and the Naïve Bayes model can be optimized by methods known to those knowledgeable in the art by minimizing the misclassification error. As another example, in the case of a logistic regression model, the cross-entropy loss between two sample classes can be used as cost function and the logistic regression model can be optimized by methods known to those knowledgeable in the art by minimizing the cross entropy loss.

In a particularly preferred embodiment of the invention the computational model in the form of a linear classifier with independent feature sampling is a Naïve Bayes classifier model.

In statistics, the term “Naïve Bayes classifier” refers to a family of probabilistic classifiers based on applying Bayes' theorem. The term “Naïve” refers to the fact, that Naïve Bayes classifiers are constructed with assumption of completed independence between each information transformed from one state to another by said Naïve Bayes computational model.

In accordance with the present invention Bernoulli distributions can be used to link a single CpG call from a Nanopore measurement of a DNA fragment to a CpG methylation rate observed during training from a group of samples from a sample class of pre-classified samples. Other examples for different types of observations and event rates are as follows:

In accordance with the present invention, binomial or Beta-binomial distributions can be used to link CpG calls from an entire genomic region from a Nanopore measurement of a DNA fragment to a CpG methylation rate observed during training from a group of samples from a sample class with pre-classified samples.

In accordance with the present invention, categorical distributions (alternatively termed: generalized Bernoulli distributions) can be used to link single genetic and/or epigenetic state calls from a Nanopore measurement of a DNA fragment to a state rate observed during training from a group of samples from a sample class of pre-classified samples. Such categorical variables include but are not limited to CpG methylation and CpG hydroxymethylation at a specific genomic position, SNP base calls (A, T, G, C) at a specific genomic position.

In accordance with the present invention, Gaussian distributions can also be used to link single genetic and/or epigenetic state calls from a Nanopore measurement of a DNA fragment to a state rate encoded in real numbers observed during training from a group of samples from a sample class of pre-classified samples. Such Gaussian variables include but are not limited to those genetic and/or epigenetic states for which measurements are output with real numbers at a specific genomic position. Such real number outputs may be encountered but are not limited for measurement technologies reporting their measurements in the form of read counts or histogram profiles. Examples for such measurement technologies include but are not limited to CHIP-seq, ATAC-seq and Highthroughput Chromosome Conformation Capture (HiC) methods known in the art.

Training of the computational model in accordance with step b) of the method of the invention may further comprise a step of evaluation (FIG. 5).

As shown in the examples herein below, the performance, i.e. accuracy and stability, of the computational model was evaluated using the Illumina Infinium array samples. To simulate a distribution of methylation events underlying the methylation rates per sample, the methylation rate for each CpG to methylated (1) or unmethylated (0) was binarized and the process was repeated 100 times per CpG resulting in 100 replicates per sample. To this end, the Bernoulli distribution was used, with parameters n=1 and p=methylation rate, resulting in a single success (methylation event=1) or failure (methylation event=0) experiment with the probability of the methylation rate.

As shown in the examples herein below, it has been evaluated if the 100 simulations of methylation events (0/1) resemble the methylation rates ([0-1]) of the respective array, by averaging across all methylation events and compared this mean to the methylation rates, showing a high similarity of the simulated and measured methylation rates.

In accordance with a preferred embodiment of the present invention, the computational model employs Bernoulli sampling and/or sparse Poisson sampling and optionally utilizes pre-determined, class-specific, genomic position-specific weights for the subsequent assignment of a sample to a sample class in step b).

The term “Bernoulli sampling” as used herein refers to a sampling process in which all members of the population (i.e. random subsets within the set of genomic positions) have the same probability of selection and the inclusion variables are jointly independent. Poisson sampling removes the restriction of equiprobability, allowing the inclusion probabilities for each member to be distinct. The term “sparse Poisson sampling as used herein” refers to a sampling process for a random subset of genomic positions wherein the restriction of equiprobability holds true and several but up to only a minority of up to 20% of all genetic and/or epigenetic information on specific genomic positions are sampled more than once. Such class-specific, genomic position-specific weights are calculated by using a statistical model to minimize the misclassification error which is estimated based on pre-classified samples evaluated with the unweighted computational model.

In accordance with the preferred embodiment described above, such sample class-specific, genomic position-specific weights for a set of genomic positions may be obtained by the determination of such weights based on parameters, such as but not limited to the per feature expectations of likelihoods of each sample class for each genomic position and the pair-wise expected differences between two or more sample classes and their variances for each genomic position.

Alternatively, in accordance with the preferred embodiment described above, the computational model may optionally utilize a pre-determined, class-specific, genomic position-specific weight for a group of sample classes for the subsequent assignment of a sample class to a sample in step b), wherein the sample class-specific, genomic position-specific weights are obtained by determination of such weights based on parameters such as but not limited to the per feature expectations of likelihood probabilities of each group of sample classes and the pair-wise expected differences between two or more groups of sample classes for each genomic position and their variances for each genomic position.

The term “per feature expectations of likelihood” as used herein refers to the expected value of the conditional probability to observe a genomic position-specific epigenetic and/or genetic information in the context of a Naïve Bayes Classifier.

The term “pair-wise expected differences” as used herein refers to a case in which between two or more sample classes or two or more groups of sample classes a genomic position-specific genetic and/or epigenetic information shows a large difference between the two or more sample classes or groups of classes. The associated genomic position-specific weight can be modified as such a large difference can encode more relevant information for distinguishing two or more sample classes or two or more groups of sample classes from each other.

The term “pair-wise expected differences” alternatively refers to a case in which between two or more sample classes or two or more groups of sample classes a genomic position-specific genetic and/or epigenetic information shows a small difference between the two or more sample classes or groups of classes. The associated genomic position-specific weight can be modified as such a small difference can encode less relevant information for distinguishing two or more sample classes or two or more groups of sample classes from each other.

In accordance with the preferred embodiment described above, the computational model optionally employs feature weighting. The term “feature weighting” as used herein relates to a procedure used to approximate the optimal degree of influence of individual features using a training set. Feature weighting may be performed by any suitable means and methods known in the art.

The term “class-specific, position-specific feature weights” as used herein relates to a list of values each of which is assigned to a) a specific sample class or a specified group of sample classes, b) a specific genomic position and c) a specific genetic and/or epigenetic feature.

As an illustrative example, such a class-specific, position-specific feature weight may have a value for CpG at a specific position of 4 for the specific sample class X. At the same time, the same CpG is associated with a class-specific, position-specific feature weight of 0.5 for sample class Y. Assuming, the CpG is determined to be methylated by nanopore sequencing, this epigenetic information will contribute 1×4=4 to the Bernoulli sampling mechanism for sample class X, but only 1×0.5=0.5 to the Bernoulli sampling mechanism for sample class Y. As a result, the epigenetic, position-specific information can contribute more or less to the scoring function of the classification mechanism depending on a) sample class or a specified group of sample classes, b) genomic position and c) specific genetic and/or epigenetic features. This can increase the diagnostic accuracy, for example, if a genetic feature is characteristic for one sample class but not for others. As a specific example, deletions of the chromosomal arms 1p/19q are considered hallmarks for IDH1/2 mutated oligodendrogliomas, but not for IDH1/2 mutated astrocytomas. Consequently, a class-specific, position-specific feature weight for information on this structural variant can increase the impact of deletions of 1p/19q chromosomal arms for the diagnosis of oligodendroglioma and thus increase the diagnostic accuracy of the Bernoulli sampling mechanism for both these two sample classes.

The use of class-specific, position-specific weights can be used to increase the predictive performance of the computational model.

In the context of brain tumor classification based on DNA methylation as shown in the examples herein below, the computational model used in the method of the invention can extract the best results even with not informative methylation calls from specific CpG positions by employing weighting steps based on the information content of a methylation call of a specific CpG position for a distinct sample class. For example, a specific CpG position might only yield unreliable CpG methylation calls because of technical reasons in the base calling step in one of the methods for determination of CpG methylation calls, this CpG position might be down-weighted by the method.

The term “CpG methylation call” as used herein relates to the determination of the presence or absence of a methyl-group attached the 5 position on the pyrimidine ring of a cytosine base. The determination of the presence or absence of methylation at a specific CpG with nanopore sequencing is based on algorithmic processes involving deep neural networks trained on nanopore current data. Such deep neural network mechanisms are employed in the basecalling software provided by Oxford Nanaopore Technologies such as Guppy5, Bonito and Megalodon (Wick et al., 2019). Consequently, the presence or absence of methylation at a specific CpG is reported by said computational mechanisms as a binary information (methylated or not methylated) with an associated probability such as e.g. a log-likelihood for this decision. Figuratively speaking, the deep neural network “calls” the presence or absence of a methyl-group attached the 5 position on the pyrimidine ring of a cytosine base and also reports how reliable this CpG methylation call may be with an associated probability estimation.

It is understood in the context of the present invention that the term “deep neural network” refers to a class of machine learning methods which have been also referred to in the literature as “artificial neural networks”. Such deep artificial neural networks are composed of artificial neurons, which are also referred to as “nodes”. Thus, such networks are composed of computational models simulating as “nodes” characteristics of communicating neural cells and are used for solving artificial intelligence and machine learning problems.

It is noted that while a specific CpG position might be not or less informative for identification of one sample class and thus be down-weighted for this sample class, the very same methylation call could be used as a highly informative data point for another sample class. The sample class specific weights are stored in sample class specific weight matrices and several methods are proposed for optimizing these matrices for the most accurate determination of the sample classes.

To accommodate the requirement that each epigenetic and/or genetic information (i.e. feature) is evaluated independently of all other epigenetic and/or genetic features utilized for the classification purpose in step b) of the method of the present invention, the weigh-matrix for each feature has to obey the same requirement.

Hence, in contrast to competing methods such as random forest classifiers, class-specific position-specific feature weights are computed for compatibility of the random order and random subset sampling procedure of e.g. nanopore sequencing and online processing of said genetic and/or epigenetic information.

In accordance with the preferred embodiment described above, the class-specific position-specific feature weights are computed adaptively and specifically for those sample classes or groups of sample classes with a relevant probability of misclassification by the computational model.

As an illustrative example, class-specific, position-specific feature weights are to be employed for a sample class for which frequently misclassifications with one or more other sample classes occur. As first step, the unweighted computational model may be utilized on a training dataset and the sample classes for which the most misclassifications occur are determined empirically. As next step, the number of sample classes for which class-specific, position-specific feature weights is determined based on those for which the most frequent misclassifications occur, e.g. the top three misclassified sample classes. Subsequently, all weights for the training dataset of genetic and/or epigenetic information available for a set of genomic positions (e.g. CpG positions) are iteratively modified for each of the selected sample classes and then iteratively, the classification result based on the modified class-specific position-specific feature weights is computed.

In accordance with the preferred embodiment described above, a Z-score for the weighted difference between the correct class and the misclassified class can be used as a Gaussian distributed approximation for the pairwise misclassification probability between two sample classes. This Z-score can be computed for each sample and for the genetic and/or epigenetic information (e.g. CpG methylation rate) of each genomic position in each sample class. For computing the optimal class-specific position-specific feature weights by means of optimization of sample class specific Z-scores and position-specific (e.g. CpG position specific) Z-scores, methods known in the art, such as the Broyden-Flecher-Goldfarb-Shanno algorithm (L-BFGS-L optimization), can be utilized.

In accordance with a particularly preferred embodiment, the class-specific position-specific feature weights are non-negative and not greater than 5. Thus, optimization algorithms such as the L-BFGS-L optimization solve a constraint optimization problem for providing the optimal class-specific position-specific feature weights.

It is understood that any other weight optimization procedure known in the art can be used to optimize the computational model's performance and that the above outlined methods are examples on how such a weight optimization could be performed.

In accordance with yet another preferred embodiment of the present invention, the genetic and/or epigenetic information of a genomic position in step b) is obtained and processed only once by said computational model and Bernoulli sampling is employed or alternatively several observations of the genetic and/or epigenetic information of a genomic position are obtained and processed by said computational model and Poisson sampling is employed.

The term “processed only once” in accordance with the preferred embodiment described above refers to the situation where a computational model transforms the genetic and/or epigenetic information on one specific genomic position from one state to another only once and does not repeat the transformation or update the transformation when more genetic and/or epigenetic information on the one or any other specific genomic positions becomes available.

In accordance with the preferred embodiment described above, based on the random subset of obtained genetic and/or epigenetic information of a genomic position, each observed information is processed by the computational model. The processing of the observed information can be seen as a scoring mechanism. The term “scoring” as used herein is the transformation of genetic and/or epigenetic information obtained from a genomic position into a likelihood of a sample belonging to each of the sample classes contained in said computational model.

The scoring is based on computing adaptively weighted log-likelihoods for each sample class contained in said computational model for the observed genetic and/or epigenetic information. Next, all weighted sample class-specific log-likelihoods are summarized and a score between 0 and 1 is computed by multiplication with a prior probability of the classes.

The term “prior probability” as used herein refers to the probability for the presence or absence of a specific sample class based on other e.g. patient specific characteristics determined by other means. For example, a hypothetical brain tumor class may have only been empirically observed at a very specific age group in childhood and no such brain tumors may have been recorded after the age of 10. In accordance with the present invention, this information can be used to determine the prior probability of a 60 year-old patient to suffer from the hypothetical brain tumor to be not existent i.e. 0.

In accordance with the present invention, this prior probability can be equal for all sample classes (also referred to as flat prior) or can be determined based on prior information e. g. from imaging data, age distribution of certain sample classes (e.g. tumor classes) and other prior features that are known about the subject and/or disease (e.g. the tumor class).

In the context of this invention, the term “adaptively weighted log-likelihoods” refers to a not equally separable sample classes based on the underlying biology. For example, IDH1/2 mutated brain tumors such as IDH1/2 mutated astrocytomas and IDH1/2 mutated oligodendrogliomas display, with respect to their CpG methylation states, many similarities. Conversely, IDH1/2 wild type gliomas such as IDH1/2 wild type glioblastomas are very distinct with respect to their overall CpG methylation patterns. Hence, to more accurately discern IDH1/2 mutated glioma classes from each other, class-specific position-specific feature weights can be utilized. Adaptive weights in this context are understood to be only integrated into the computation of the feature specific log-likelihoods by the computational model for the sample classes with mutations in the IDH1/2 gene. Adaptive weights can be employed for groups of similar sample classes with respect to their overall unweighted classification performance or can be triggered by the detection of characteristic genetic and/or epigenetic features during the process of sequencing, as e.g. during nanopore sequencing.

In a further preferred embodiment of the present invention, the number of genomic positions in said random subset of genomic positions of step a) increases continuously, thereby providing the genetic and/or epigenetic information in the form of a data stream and the computational model in step b) processes the genetic and/or epigenetic information in the form of said data stream, thereby updating the assignment at the same rate or close to the same rate at which more genetic and/or epigenetic information become available through said data stream and processes all genetic and/or epigenetic information up to the timepoint of the update.

As the computational model used in the method of the invention can score each of the sequentially obtained genetic and/or epigenetic information of a genomic position by computing adaptively for each sample class weighted log-likelihoods for the observed genetic and/or epigenetic information given a sample class from the number of pre-defined sample classes and all weighted sample class-specific log-likelihoods are summarized, this process can continuously update a score for the probability that a sample belongs to a specific sample class as more and more genetic and/or epigenetic information on genomic positions of the sample are obtained by the nanopore sequencing process.

Accordingly, the method of the present invention allows a real-time analysis of sequencing data from an ongoing nanopore sequencing process.

In a preferred embodiment of the invention, the computational model uses a function that assigns to data sample x=(x₁, . . . , xₙ) a class label ŷ for class k as follows:

$$\hat{y} = arg {max}_{k ∈ \{ 1 , ... K \}} P ( {C}_{k} ) \cdot {\prod }_{i = 1}^{n} {p}_{{k}_{i}}^{{x}_{i}} \cdot {( 1 - {p}_{{k}_{i}} )}^{( 1 - {x}_{i} )} ,$$

xᵢ=measurement at i-th position of sample x within n positions
pₖ_{ᵢ}=probability of the measurement at i-th position being from class k (centroid of class k at position i)
P(Cₖ)=prior probability of class k (e.g. relative frequency of class k in the training data set).

The method of the invention is at least partly performed digitally and therefore in some embodiments the genetic and/or epigenetic information is provided in computer-readable form, wherein step b) of the method of the present invention is performed in-silico, preferably on a digital computer.

In a yet further embodiment in accordance with the present invention, the method of the invention is a computer-implemented method.

The invention is herein described, by way of example only, with reference to the accompanying drawings for purposes of illustrative discussion of the preferred embodiments of the present invention.

Unless otherwise defined, all technical and scientific terms used herein have the same meaning as commonly understood by one of ordinary skill in the art to which this invention belongs. In case of conflict, the patent specification including definitions, will prevail.

Regarding the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all appended claims.

### The Figures Show:

In the context of the figures and examples the (Bernoulli Naïve Bayes) computational model of the present invention is also at times referred to with the term “MethyLYZR” for brevity reasons.

FIG. 1: (A) Standard visualization of a Naïve Bayes Network with the associated mathematical formula; (B): Visualization of a Naïve Bayes Network with corresponding terms from the present invention; (C): Visualisation of a larger Naïve Bayes Network with a larger number of nodes with input from a nanopore sequencing process into the class probability estimation of the Naïve Bayes algorithm. Note that black and white nodes represent a binary input exemplifying for example CpGs at distinct genomic positions, which have been determined to be methylated or unmethylated; (D): Visualisation of two defined subsets from the larger Naïve Bayes Network in FIG. 1C. Note that each network contains only a subset of nodes from FIG. 1C with input from the same nanopore sequencing process into the class probability estimation of the Naïve Bayes algorithm is present. Note also, that both subsets do not overlap with their input nodes; (E): Visualisation of a third, random subset from the larger Naïve Bayes Network in FIG. 1C. Note that a random subset with input from the same nanopore sequencing process into the class probability estimation of the Naïve Bayes algorithm is exemplifying a nanopore sequencing process as a stochastic source; (F): Visualisation of a fourth, random subset from the larger Naïve Bayes Network in FIG. 1C. Note that a random subset with input from the same nanopore sequencing process into the class probability estimation of the Naïve Bayes algorithm is exemplifying a nanopore sequencing process as a stochastic source; (G) Optimized library preparation protocol takes less than 1 hour. Timeline for the application of the method of the invention in the diagnosis and/or classification of a disease in a subject based on the genetic and/r epigenetic information of a sample obtained from a subject, including the steps of sample preparation, library preparation and nanopore sequencing.

FIG. 2: Processing of reads as they come of the sequencer. As the raw reads come of the Nanopore sequencing device, they are saved as fast5. Batches of reads are then processed using the Megalodon software, which consists of the basecalling, alignment and methylation rate calling (using a neural network). The alignment is then saved as a BAM file and can be used for copy number variation (CNV) detection. The methylation rates are used in our Bernoulli Naïve Bayes algorithm and used for prediction of the tumor diagnosis.

FIG. 3: Throughput of Nanopore sequencing within 5 hours.
(left) Using the MinION to sequence a RAD library, results of on average 15 mega bases in 5 minutes sequencing time and about 160 mega bases within 30 minutes. The black line denotes the mean and the shaded area the 25 and 75% quantiles.
(center) This translates to a linear relationship between sequencing time and number of CpGs in the first hours and on average 500 CpGs within the first 2 minutes and 2-5k CpG within the 30 minutes. The black line denotes the mean and the shaded area the 25 and 75% quantiles.
(right) Summary of multiple flow cells shows that 30 minutes of sequencing time with a MinION and even less with a PromethION are sufficient to sequence 5000 CpGs that are also present on the Illumina Infinium MethylationEPIC BeadChip. Boxplots: the centerline is median; boxes, first and third quartiles; whiskers, 1.5× inter-quartile range; data beyond the end of the whiskers are displayed as points.

FIG. 4: Concept of model training and diagnosis prediction.
(top) Illumina Infinium HumanMethylation450 BeadChip data are used to calculate kernels/centroids representing the different tumor classes. Here, the methylation rates of these classes are used for model training.
(bottom) Nanopore sequencing data from a tumor biopsy are usually poor in coverage, therefore rather representing methylation event than methylation event. These events are then used in the Naive Bayes model, to predict in real-time the tumor diagnosis.

FIG. 5: Model training and application of the computational model of the invention for streaming interpretation of nanopore sequencing signals for the classification of brain tumor samples based on DNA methylation patterns.

FIG. 6: Generation of test-data for performance evaluation. The methylation rates of all Illumina Infinium HumanMethylation450 BeadChip array samples were in silico downsamples to resemble low coverage Nanopore sequencing methylation events. Using a Bernoulli distribution with the methylation rate as probability a 0 or 1 obtained for each CpG. To increase the number of test data sets and also cover the variety of methylated/unmethylated molecules that could have been underlying the probe, this process was repeated 100 time per brain tumor array.

FIG. 7: Prediction accuracy using in silico down-sampled array data. Using 2000 randomly selected CpGs for each of the test data sets shows a very high precision for all tumor classes. All classes show an accuracy of higher than 95% when considering the top 2 predicted classes.

FIG. 8: Confusion matrix of in silico down-sampled array data. Confusion matrix showing the rate of misclassification of a tumor class. The great majority of tumor classes are predicted correctly with a very high accuracy of greater than 95% for each class.

FIG. 9: Example of performance of a weak Nanopore sequencing run (IDH wild-type glioblastoma). Matrix with color coded prediction probability for each tumor class for tumor class prediction after 5, 10, 15, 20, 25, 30, 45, 60, 150, 720, 1440 and 2880 minutes of sequencing. In this run, the library performed not very well and only 600 CpGs were sequenced after 30 minutes. During the first 30 minutes, the prediction probabilities were rather low and distributed across multiple tumor classes. After 30 minutes of sequencing the diagnosis became stable and the correct IDH wild-type Glioblastoma was predicted.

FIG. 10: Example of performance of an average Nanopore sequencing run (IDH wild-type astrocytoma). Matrix with color coded prediction probability for each tumor class for tumor class prediction after 5, 10, 15, 20, 25, 30, 45, 60, 150, 720, 1440 and 2880 minutes of sequencing. In this run, the library performed decent and 5000 CpGs were sequenced after 30 minutes. During the first 5 minutes, the prediction not yet correct. However, already after 10 minutes of sequencing the diagnosis became stable and the correct IDH wild-type Astrocytoma was predicted.

FIG. 11: Determination of class separability. Genome-wide methylation rates from Illumina 450K Methylation arrays for three groups of brain tumor samples have been plotted on two dimensions (um[, 1] and um[,2]) with a dimension reduction technique (UMAP, left graph). A priori determined class labels have been color-coded and mapped on the respective samples. The graph demonstrates that most samples can be separated into three a priori defined sample groups on this two-dimensional UMAP plot as shown in the right graph. In this geometric example, intuitively, the lines suggest potential, algorithmically defined sample class boundaries. From the right graph, it can be easily appreciated that a linear separating line can separate all O IDH samples from all A IDH and A IDH HG samples, thus indicating two well separable sample classes based on CpG methylation data in this example. In contrast, no simple separating line can be found separating all A IDH from all A IDH HG samples, thus suggesting two difficult to separate sample classes based on CpG methylation data in this example. In the last panel, the use of two centroids per class is visualized. Two or more centroids per sample class can improve the separability of difficult to separate sample classes with a high misclassification rate.

FIG. 12: Correlation between different devices, i.e. 450k array vs Nanopore measured methylation rates shows that a general bias occurs, for which a specific normalization and correction might be applied to correct it.

FIG. 13: UMAP plot demonstrating the two dominant subclusters relating to male (left) and female (patients) right in the methylation dataset. Red nodes denote control subjects, black nodes denote patients with rheumatoid arthritis.

FIG. 14: UMAP plot demonstrating the two dominant subclusters relating to male (left) and female (patients) right in the methylation dataset. The same UMAP as in FIG. 13 is shown. In this figure red nodes denote male controls and RA patients, black nodes denote female controls and RA patients.

FIG. 15: Performance of the computational model for the distinction between subjects with and without rheumatoid arthritis according to the present invention over the inclusion rate and in regard to the test- and training data subsets.

FIG. 16: Snapshots from a continuous video recording of the intraoperatively sequencing of tumor sample IEG 108 with the associated time stamps and steps in the sequencing process displayed in the photographs. The first snapshot in the upper left depicts the ongoing ultra-rapid DNA extraction at time stamp 19 minutes 45 seconds. The second snapshot in the upper right depicts the ongoing DNA quality control at time stamp 22 minutes 24 seconds. The third snapshot in the lower left depicts the ongoing nanopore library preparation and flow cell loading at time stamp 31 minutes 17 seconds. The fourth snapshot in the lower right depicts the online analysis of the nanopore sequencing data with a computational model according to the present invention at time stamp 45 minutes 17 seconds.

The examples illustrate the invention:

### Example 1: DNA Extraction and Preparation of Sequencing Library

### a) DNA Extraction of Fresh Brain Biopsies

DNA extraction was performed using the QIAamp Fast DNA Tissue Kit (QIAGEN, Hilden, Germany) following the manufacturer's protocol with minor modification. Briefly 15 mg of brain tissue was weighed and transferred into a tissue disruption tube. Tissue lysis was done by adding 265 μl digestion buffer mix followed by sample homogenization at 45 Hz for 2 min using the TissueLyser LT (Qiagen). Protein and RNase digestion was subsequently carried out at 56° C. for 7 min and 1000 rpm in a Thermomixer. Digested sample was supplemented with 265 μl buffer MVL and homogenized by pipetting. Precipitated DNA mixture was loaded onto a QIAamp mini spin column and centrifuged at 20,000×g for 1 min followed by two wash steps with 500 μl buffer AW1 and AW2 at 20,000×g for 30 sec. Residual ethanol was removed by centrifugation at 20,000×g for 2 min. Elution of DNA was done using 50 μl of preheated (56° C.) nuclease-free H₂O for 1 min following a centrifugation step at 20,000×g for 1 min. DNA quantification was carried out using the Nanodrop One (Thermo Fisher Scientific).

### b) Preparation of ONT Sequencing Libraries

Preparation of low-throughput rapid libraries the SQK-RAD004 Rapid Sequencing Kit was used following the manufacturer's recommendation with minor modifications. Briefly two times 600-700 ng of QIAamp Fast DNA Tissue Kit extracted DNA was transferred into a 0.2 mL PCR tube and adjusted to a total volume of 7.5 μl with nuclease-free H₂O. Each sample was supplemented with 2.5 μl of fragmentation mix (FRA) and placed into a preheated (30° C.) Thermocycler (Doppio, VWR). Fragmentation was immediately performed at 30° C. for 1 min following a heat inactivation step at 80° C. for 1 min. Attachment of the rapid sequencing adapter (RAP) was carried out for 10 min at room temperature. Meanwhile two MinION flow cells (Oxford Nanopore Technologies, FLO-MIN106D R9.4) were primed following the manufacturer's protocol using room temperature equilibrated priming mix. Final libraries were supplemented with 34 μl sequencing buffer (SQB), 25.5 μl loading beads and 4.5 μl nuclease-free water and samples were immediately loaded onto the SpotON port.

### Specific Protocol Optimization:

In order to allow intraoperative molecular diagnosis from brain biopsies within 1 hour several protocol modifications have been introduced. Special attention was paid to the selection of DNA extraction and sequencing library preparation kits to enable quick processing times and minimal handling steps with sufficient sample purity. The respective protocol adaptations are discussed below:

### i) Tissue Weigh & DNA Extraction Kit:

The QIAamp Fast DNA Tissue Kit allows quick DNA extraction from various tissue samples up to 25 mg of starting material. It was found that brain biopsies collected from cancerous tumor tissue can vary substantially with regard to DNA amount and purity based on tumor localization and cellular composition. Thus, the tissue weight needed for sufficient DNA yield with acceptable purity for nanopore libraries was empirically determined and was found to be 15 mg. However, also lower input amounts were examined. It was found that 5 mg of tumor tissue material in this case is enough to ensure successful preparation of sequencing libraries. This allows molecular diagnostics even from low-input material obtained during stereotactic brain surgery.

### ii) Sample Digestion and DNA Purity:

Large amounts of biopsy sample (15-25 mg) require longer incubation time for protein and RNA digestion. In turn less input of brain tumor sample can benefit from shorter digestion times. In order to develop an intraoperative workflow within 1 hour the sample digestion time was reduced from 10 min down to 7 min at 56° C. for tissue weights of 15 mg and below. This helps to reduce total DNA extraction time from 18 min to 15 min (11%). It was found that reduced digestion time has only minor effects on DNA purity measured by Nanodrop (260/280 and 260/230 ratios) and give comparable sequencing results for the first hour of nanopore sequencing.

### III) DNA Elution:

Silica containing spin columns are widely used in today's routine DNA or RNA extraction workflows. Efficient DNA elution is mainly driven by ionic strength, pH, temperature and DNA fragment size bound to the silica membrane. In order to reduce potential contaminants of the kit containing elution buffer that can't be replaced during the preparation of the sequencing library (e.g. EDTA) nuclease-free H₂O pH 7-8 was used. In addition, equilibration of H₂O was conducted at 56° C., which helps to increase DNA yield especially for longer DNA fragments resulting in high DNA concentration needed for the rapid library preparation kit.

### iv) DNA Amount for Sequencing Library:

Protocols for the preparation of DNA long-read sequencing libraries differ substantially with respect to DNA input amount, DNA fragment length, sequencing performance and yield. Those protocols have mainly been developed using well-defined lambda control DNA that is characterized by a distinct DNA fragment length. However, such control sample is insufficient to recapitulate more complex genomic DNA samples. Saturation of the fragmentation reaction is key for obtaining high amounts of molecules labeled with the transposase adapter (i.e. the tagging adapter). Therefore, the specific input amount of DNA sample obtained from the QIAamp Fast DNA Tissue Kit was evaluated and it was found that the sequencing library benefits from increased DNA input ranging from 600-700 ng in combination with increased rapid adapter attachment incubation. These changes were able to increase pore occupancy (ratio of “in strand” to the sum of “in strand” plus “single pores” after one hour) significantly (up to 20%) compared to standard rapid library preparations resulting in higher sequencing throughput within the first hour of sequencing.

### v) Rad Attachment:

Sequencing of DNA fragments can only take place if those molecules are coupled to a motor protein which is bound to the rapid sequencing adapter (RAD). Sufficient attachment of the rapid sequencing adapter (RAD) is therefore vital for obtaining good sequencing performance. It was found that increased incubation time (10 min) for RAD attachment increases the quantity of molecules coupled to the respective sequencing adapter leading to an increase in pore occupancy and better sequencing yield.

### vi) Flow Cell Priming:

Nanopore flow cells are treated with a priming mix prior loading of the final sequencing library. The priming mix contains flush tether (FLT) molecules that can bind in close proximity to the nanopores. Additionally, they are mediating the binding of sequencing molecules to the protein pores. However, the original rapid library preparation protocol recommends storing the priming mix on ice prior to use which results in cooling of the sensor array. Consequently, the flow cell needs to be heated in order to reach the desired temperature to initiate the MUX scan (sensor multiplexing). This process is relatively time consuming and can take up to 5 min. To enable a faster start of sequencing the priming mix was equilibrated to room temperature reducing the time from sample loading to sequencing from 5 min to 1 min most time effective sequencing.

### Example 2: Cohort and Sample Generation

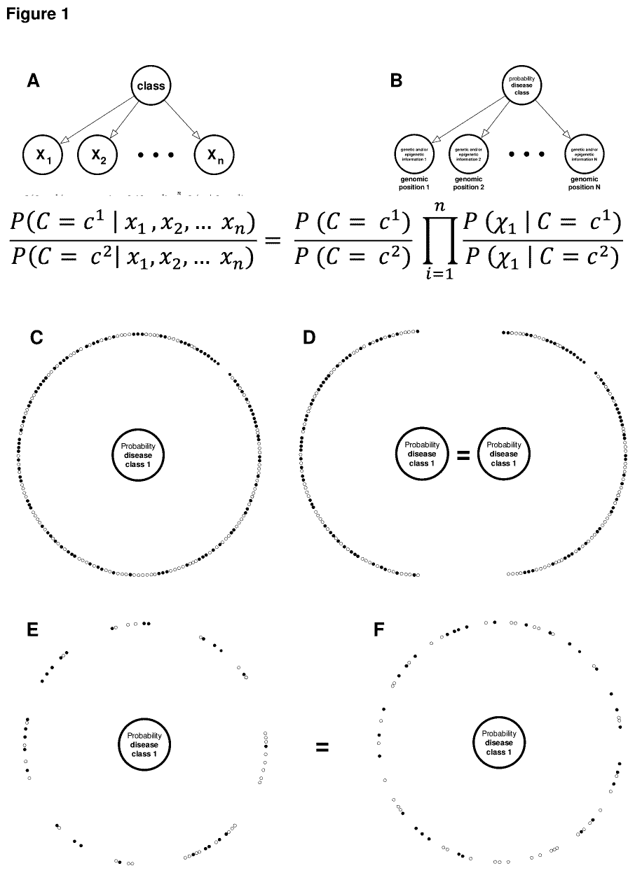

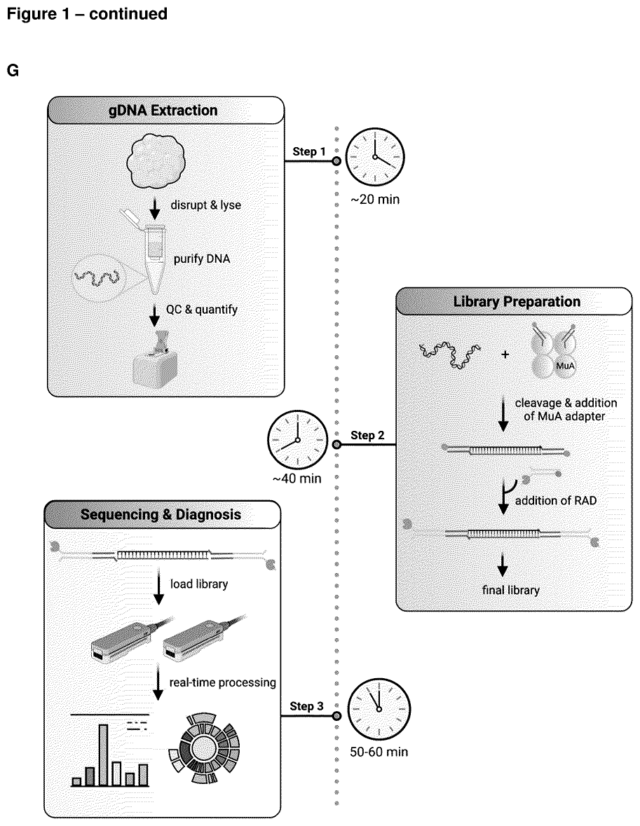

During brain tumor surgery and treatment, time in the operating room and also until diagnosis is a critical feature. To enable real-time based molecular diagnosis, two technical limitations need to be overcome: One is the throughput of the underlying methods needs to be sufficiently high, to allow for a confident diagnostic call, within a very short window of time. The second is decisive in how much time this is, which is the time needed to prepare the biological material. To optimize the time required for these two time-wise linked steps, 101 flow cells with brain tumor samples were sequenced using Nanopore long-read sequencing from 12 different brain tumor subclasses (Table 1). The time requirement of the two main library preparation kits combined with the usage of the two main ONT sequencers, namely the MinION and the PromethION, that notably differ in throughput was benchmarked. Using Oxford Nanopores rapid library preparation kit (SQK-RAD) drastically reduced the time from receiving the tissue probe until the beginning of sequencing to effectively 40 minutes by taking advantage of a fast two-step protocol (FIG. 1g). First, a MuA transposase is added to the sample resulting in a rapid fragmentation of DNA with the simultaneous attachment of a transposase adapter. Secondly, a sequencing adapter (RAD) containing a motor protein, is attached leading to a functional sequencing library. Subsequent sequencing of RAD libraries shows the expected higher throughput in nucleotides sequenced when using the PromethION (FIG. 3). When focusing on CpGs sequenced this difference decreases and allows for sequencing of 200-500k and more than 750k CpGs (that are also located on the Infinium MethylationEPIC BeadChip array) within 5 hours using the MinION and the PromethION, respectively (FIG. 3). To estimate the time required until an initial molecular diagnosis could be confidently obtained, the time elapsed was measured until 2000 and 5000 CpGs were sequenced. Surprisingly, it was found that both nanopore platforms are capable of sequencing up to 5000 CpG within less than 20 minutes for most of the samples (FIG. 3). When combining the extremely short library preparation time with the time used for sequencing of a minimal number of CpGs needed for classification, a time period of less than 1 hour from receiving the tumor sample until having sequenced the required minimum number of CpGs for brain tumor class prediction was reached. Due to the more extended library preparation time of the ligation sequencing kit LSK (evaluated on both MinION and PromethION platforms, n=46), these samples are only used for classification benchmarking, though are not considered real-time capable despite showing sufficient throughput (FIG. 3).

TABLE 1
Number of flow cells sequenced per device
and library preparation kit per tumor class

| | | MinION | |
|---|---|---|---|
| Tumor class | MinION | (RAD) | PromethION |
| Ependymoma | | | 2 |
| IDH wildtype Glioblastoma | | 12 | 21 |
| IDH-mutant Astrocytoma | | 11 | 10 |
| IDH-mutant Astrocytoma/pilocytic | | | 1 |
| Astrocytoma | | | |
| IDH-mutant Glioblastoma (no | | 1 | 1 |
| prediction class) | | | |
| Low grade glioma | | 1 | 1 |
| Lymphoma | | 1 | 1 |
| Oligodendroglioma | | 12 | 13 |
| Other Glioma | 2 | 1 | 3 |
| Pilocytic astrocytoma | | | 1 |
| Plexus tumor | | 1 | 1 |
| IDH wildtype glioma with H3 | | 1 | |
| molecular features | | | |
| Diagnosis could not be stablished | 1 | 2 | 1 |

### Example 3: Classifier Development

Recently published cancer classification methods from DNA methylation data rely on information from a fixed number of positions, for example, as obtained from Illumina Infinium HumanMethylation450 or MethylationEPIC BeadChip arrays. However, this requirement is usually not fulfilled when applied to real-time sequencing within a very short period. To overcome this methodological challenge, the method of the invention was developed (using a flexible Naïve Bayes classifier) to predict the underlying cancer type from sparse, shallow coverage Nanopore sequencing data (FIG. 5). Due to the nature of a Naïve Bayes classifier, missing values are not relevant or problematic and thus based on any combination of present information, a sample classification is possible. It is important to note that in this specific use case, Nanopore sequencing rather results in the binary event information of methylated or unmethylated for a covered CpG than an average estimate across the entire cell population. In brief, every CpG is evaluated independently by calculating the likelihood that it results from any of the trained classes, given that the methylation rate at this position reflects precisely the probability of this CpG being methylated in the corresponding class. Finally, the likelihoods for all observed CpGs are combined to calculate the overall likelihood (posterior probability) for the sequenced sample to stem from the corresponding tumor class.

A publicly available Illumina Infinium array data was used to train the classifier distinguishing several brain tumor classes (n=43, Capper et al. 2018, Orozco et al. 2018). Methylation rates from n=2848 patients were used for training, with the beta values representing the mean methylation information for each CpG based on a large number of tumor cells within each sample (FIG. 4, top). When applying the trained classifier to a Nanopore sequencing sample, the posterior probability is calculated for that specific sample to originate from each training class, given the methylation information of a subset of CpGs present in the training set (FIG. 4, bottom). The output of our algorithm is the posterior probabilities for a sequenced probe to stem from any of the training tumor classes.

To train the Bayes classifier on tumor types with a distinguishable methylome but simultaneously only recover clinically and specifically surgically relevant diagnosis, we summarized several tumor classes within a diagnostic class. Technically, centroids (kernels) were trained per tumor class (FIG. 5) and subsequently directly translated the predicted classes into the broader diagnostic classes based on which the evaluation was done.

### Example 4: Classifier Validation

For validation of classification accuracy, a comprehensive validation strategy was conducted by repeatedly (×100) binarizing each of the beta-values (∈[0, 1] e.g. Bernoulli sampling with success probability equals beta rate) of each Illumina Infinium HumanMethylation 450k BeadArray sample to resemble the methylation events (∈{0, 1}) as obtained from Nanopore sequencing within 30 minutes of sequencing (FIG. 6). These 100×2,801 simulated samples were randomly sub-sampled to a sparse representation (n=200, 500, 2000 and 5000) of the training CpGs and subsequently used as input for classification.

Comparison of actual and predicted diagnosis resulted in an estimated error rate of less than 5% on average over all tumor classes, indicating a high discriminating power (n=2000 CpGs, FIG. 8). The majority of classes were predicted correctly in the simulations. The small number of classes in which misclassifications occurred were within groups of histologically and biologically closely related tumor classes. Closer inspection of the prediction accuracy reveals that for 42 out of 43 classes, the accuracy is already above 90% when considering the most likely predicted class and above 95% for all classes, when taking the top 2 classes into account (FIG. 7).

### Example 5: Prediction of Diagnosis Based on Nanopore Data

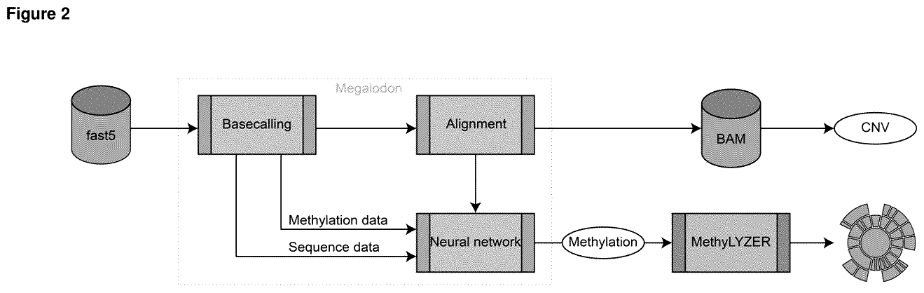

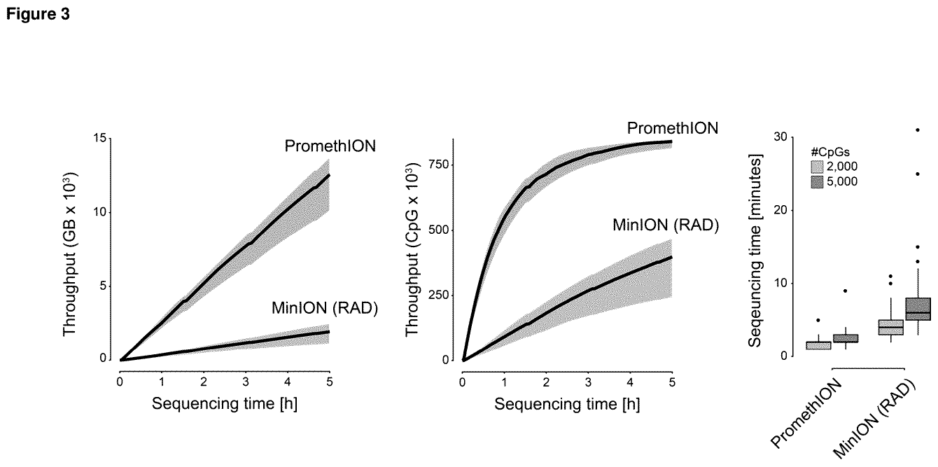

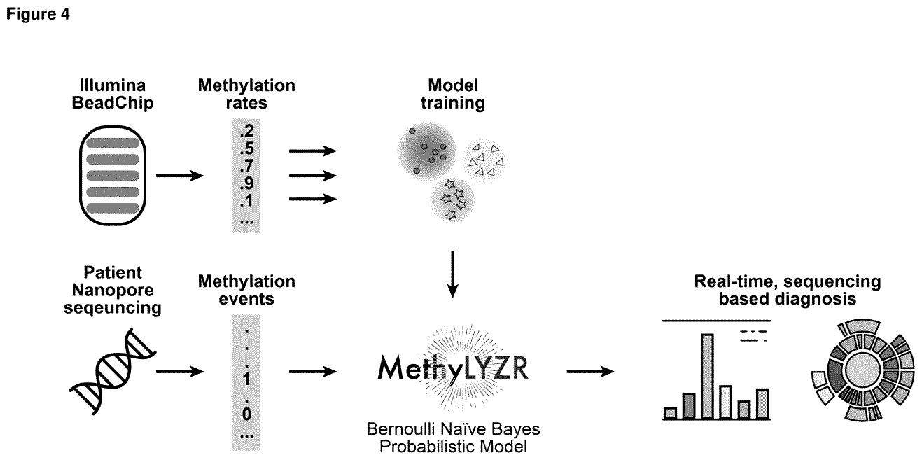

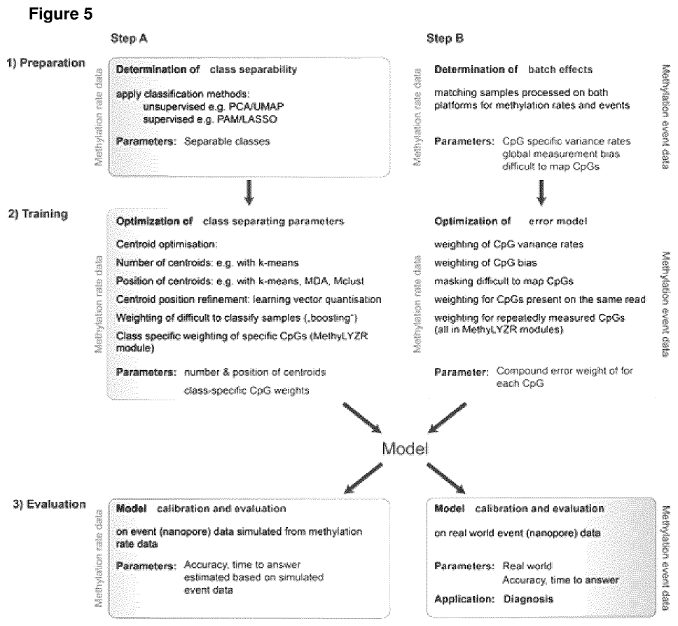

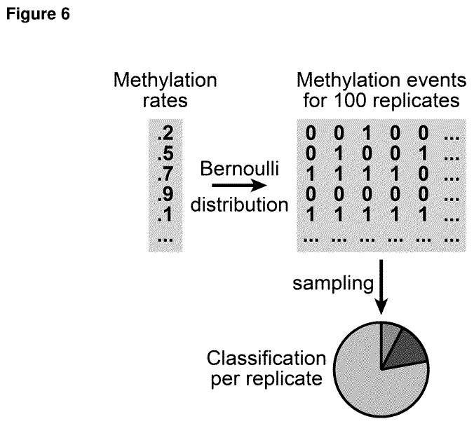

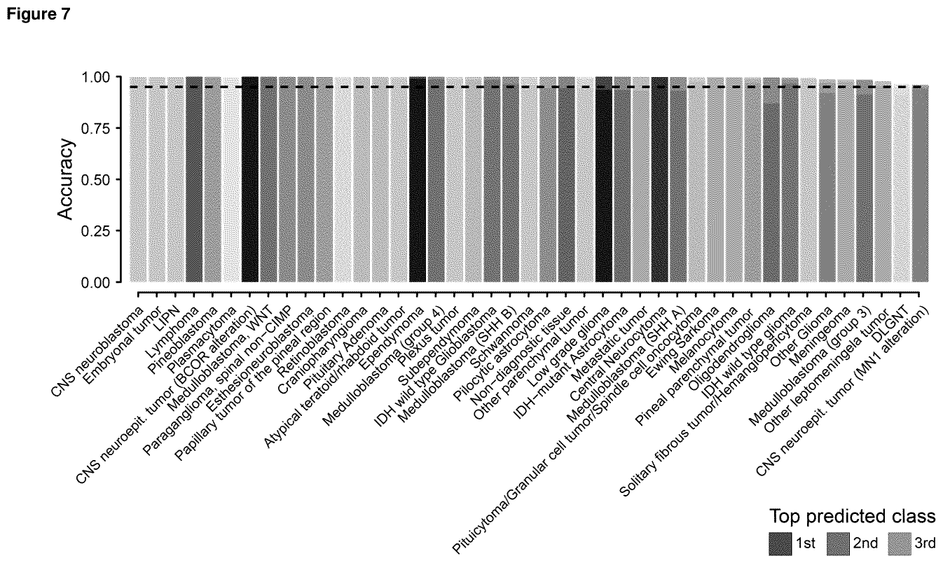

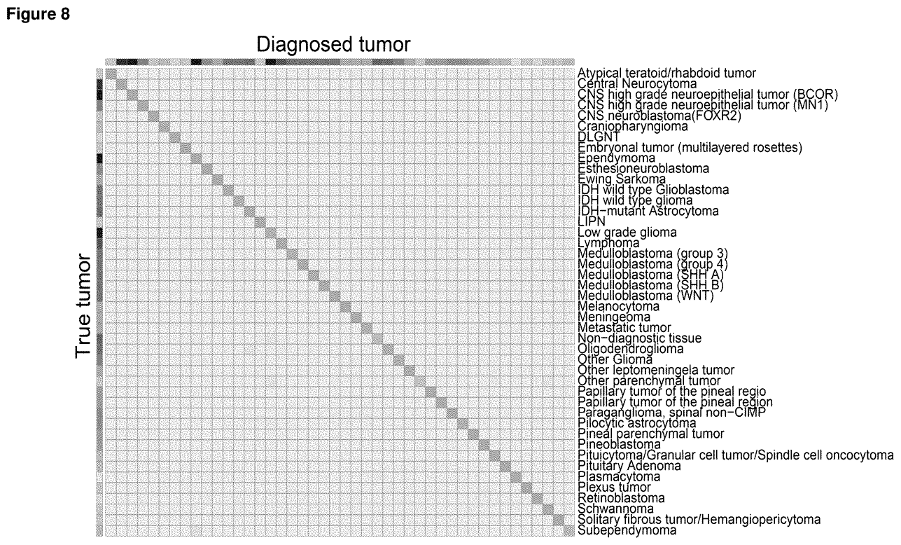

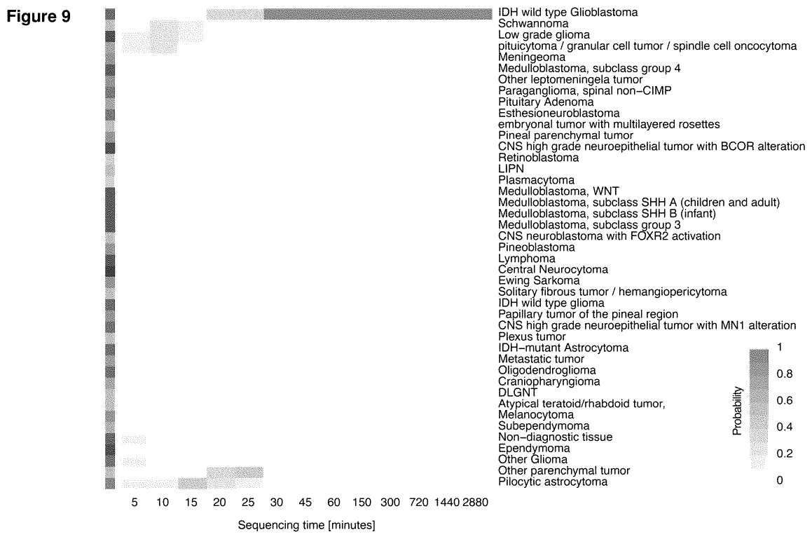

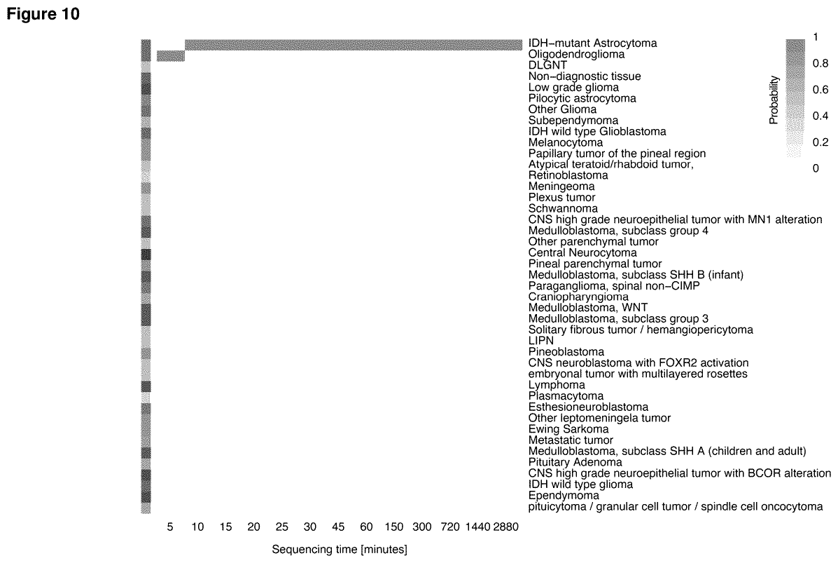

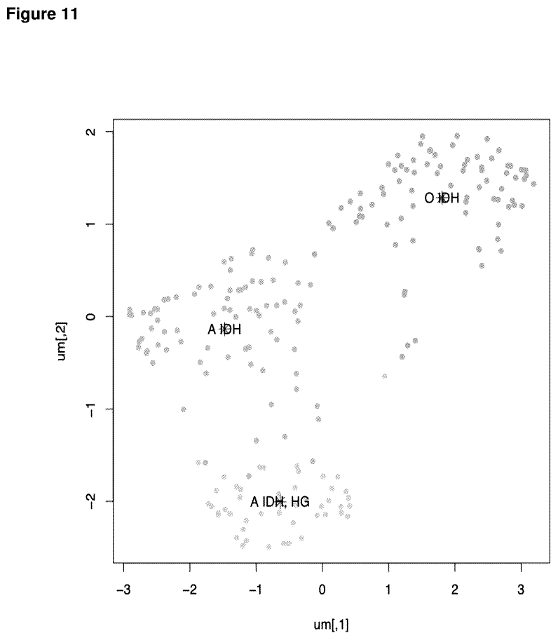

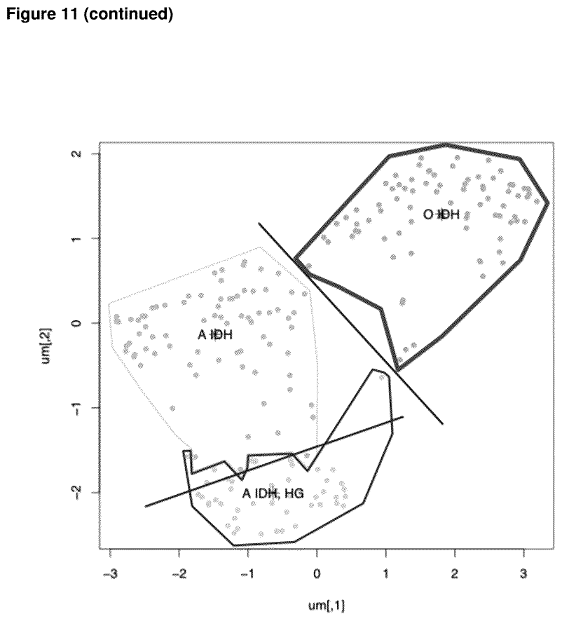

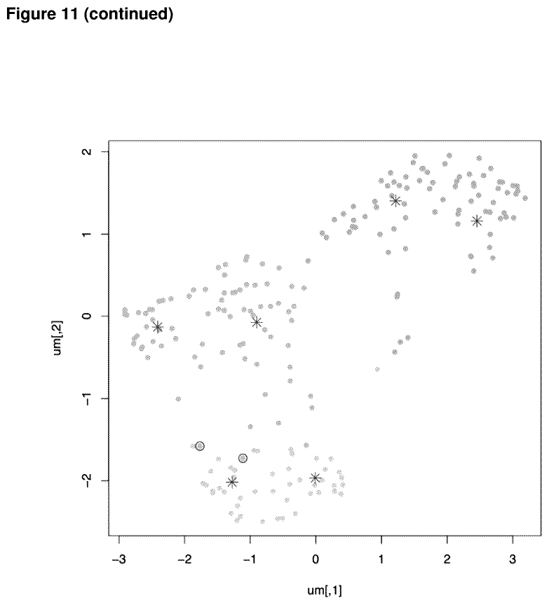

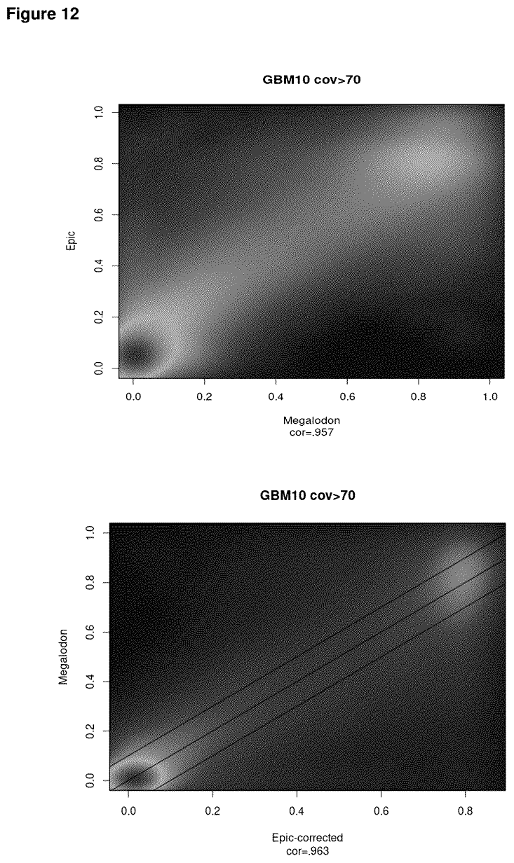

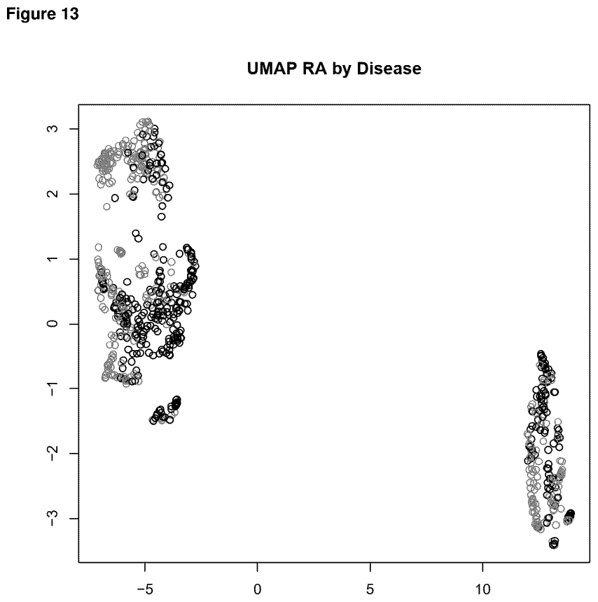

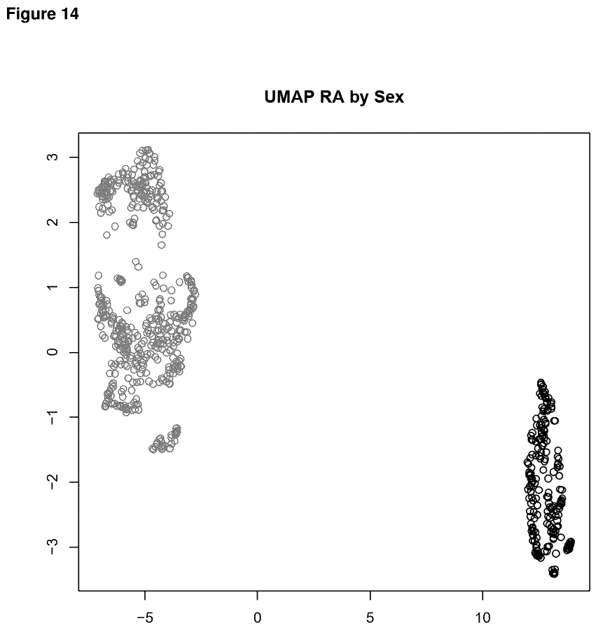

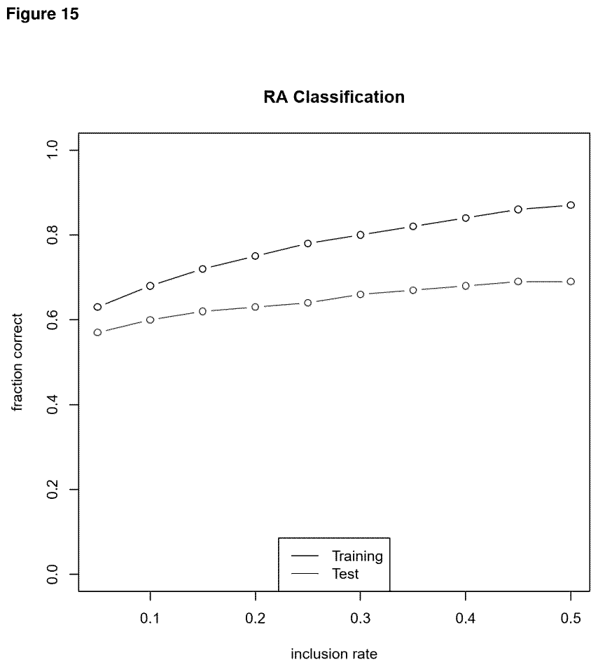

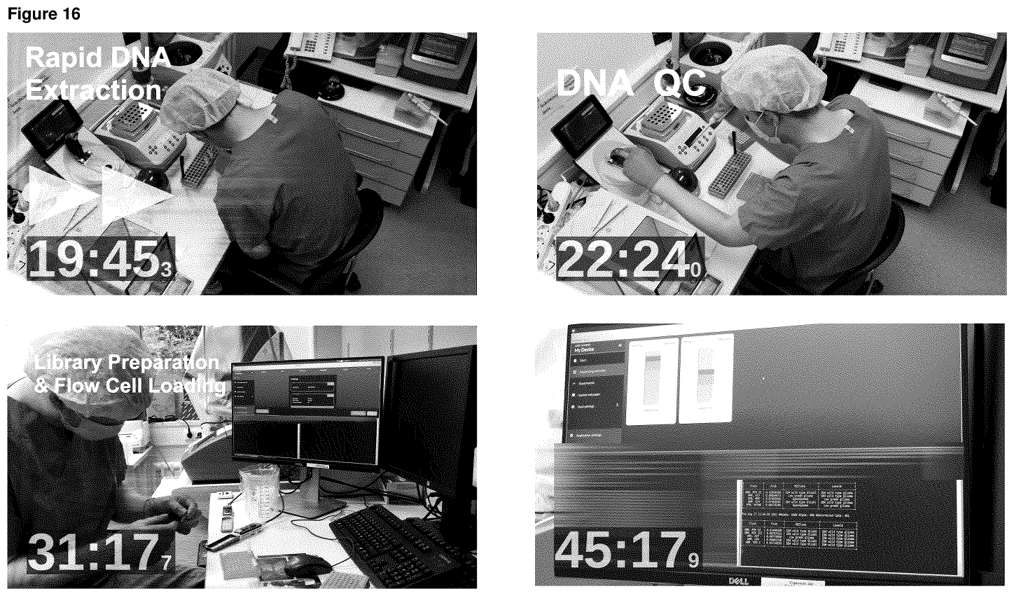

To enable real-time prediction of the clinical diagnosis, developed a streamlined processing pipeline of Nanopore raw data was developed (FIG. 2). In brief, once the nanopore sequencing starts, megalodons analysis software is initialized and incoming sequencing data are subsequently processed in small batches of 1,000 reads. The resulting data are further processed by taking advantage of a neural network that outputs a methylation probability for each CpG in any given sequencing read. In addition, these steps allow for building a CNV profile from the aligned data, which is particularly important for predicting the 1p19q status.

Next, the computational model reduces the methylation output for CpGs included in the training dataset to a maximum of 2 CpGs per read (random selection) and collapses the methylation information of CpGs covered by more than one read. This condensed information is then used as input for the Naïve Bayes classifier to predict the underlying brain tumor class. In parallel, further batches are processed automatically and analyzed in real-time, resulting in increasing accuracy of tumor classification over time.

When evaluating the precision of our predictions as a function of time (focusing on the RAD kit with MinION sequencing), it was found that already after 5 minutes of sequencing, 80% of the samples were predicted correctly (on average 940 [133-2929] CpGs sequenced). The most substantial increase in predictive power across all samples was present within the first 20 minutes (on average 5000 [576−10572] CpGs sequenced), such that there was only one sample sequenced for which an increased waiting time would have improved the diagnosis. For a subset of only 17.5% of all samples, the diagnosis differed from the diagnosis of the pathologists; However, the general comparison of predicted and diagnosed tumor class supports the general high accuracy. Focusing on two examples—one being the rare exception of a low-performance library or flow cell and the other being a representative sample of an average, good library and flow cell, it is highlighted, that even in case of weak performance, 600 CpGs were sequenced within 30 minutes, resulting in the correct diagnosis (FIG. 9) In line with this, an average flow cell with more than 5000 CpGs sequenced within 30 minutes, already predicts the final tumor class with a high probability earlier than after 30 minutes (FIG. 10).

Finally, to account for the randomness in CpG sequencing order and ensure that the results after 20 minutes were reproducible, for each sample a random selection of the same number of reads as obtained in the actual sequencing experiment (N=100 simulations per sample) was simulated. Approximately the same number of CpGs were obtained for each simulation, showing that no strong read-length effect was present throughout our sequencing runs, suggesting towards a stable sequencing outcome independent of the subset of reads analyzed.

### Example 6: Diagnosis of Brain Tumors Based on cfDNA from Cerebrospinal Fluid

While sequencing of DNA fragments with a read length of several kilobases has proven to be effective in the context of workflows building upon the present invention (as demonstrated in Example 5: Prediction based on Nanopore data), the application of computational models according to the present invention on cell-free DNA (cfDNA) was performed and the results were evaluated.

cfDNA is challenging to sequence, as substantially shorter DNA fragment lengths (100-200 bp) are regularly obtained from liquid biopsies. It was found that cfDNA obtained from brain tumor patients' cerebrospinal fluid (CSF) is particularly suitable for a same-day diagnostic approach. A lumbar puncture and sampling of 5-10 ml of CSF from a brain tumor patient in combination with nanopore sequencing and computational models according to the present invention enables the diagnosis of a brain tumor class from the said liquid biopsy. Said diagnostic procedure can enable the implementation of specific neurosurgical procedures such as but not limited to the extent of resection and optimal anatomical approach and may enable the initiation of targeted chemotherapeutic therapies already intraoperatively. Also, said diagnosis and/or classification may enable the diagnosis of inoperable brain tumors as well as relapse monitoring after neurosurgical intervention with a lumbar puncture as less invasive procedures than burr hole biopsy or open craniotomy.

Analysis of cfDNA from CSF is challenging with competing methods, as CSF usually contains only minimal amounts of cfDNA. Also, the extracted DNA may be contaminated with DNS originating from non-tumor cells such as inflammatory cells. The present invention allows the diagnosis of relevant brain tumor classes based on methylation and copy number variation profiling but also will be extended in the future to incorporate additional genetic and/or epigenetic information, which can be obtained from alignment of cfDNA nanopore reads to a reference genome such as but not limited to position specific cfDNA fragment size, copy number variation and fragment end motifs.

Due to the relatively low abundance of cfDNA from CSF (typically <50 ng/ml), sample collection was either done by using 5 mL DNA low bind tubes or customized container/magnetic beads with a modified surface that allows adsorption of DNA under specific binding conditions (polymer mediated enrichment). Briefly, 5-10 mL of fresh CSF was subjected to cfDNA extraction by using the QIAamp Circulating Nucleic Acid kit (Qiagen), NucleoSnap cfDNA kit (Macherey-Nagel), or PME free-circulating DNA Extraction kit (IST innuscreen) following the manufacturers' recommendation. Extracted DNA was quality controlled and quantified on an Agilent BioAnalyzer device using the High Sensitivity DNA kit. Next, a total of 10-30 ng of cfDNA was subjected to sequencing library preparation by taking advantage of the ligation sequencing kit SQK-LSK109 (ONT) with minor protocol modifications. Briefly, 1.5× Ampure XP beads were used for DNA binding and subsequent wash steps and incubation time for the ligation of sequencing adapters was extended to 20 min, and 5-10 ng of the resulting sequencing library (35-70 fmol) was loaded onto an R9.4.1 MinION flow cell.

While the molecular sequencing procedure is ongoing, the resulting data is continuously analyzed online as it becomes available from the nanopore sequencer. For basecalling, Guppy 6.1.5 was used, which utilizes the Remora methylation calling algorithm. After base-calling, reads were mapped to a human reference genome (GRCh38.p13) using minimap2. All CpGs with an associated determination of their epigenetic state by Remora were subsequently filtered to the CpG-sites present on an Illumina Infinium HumanMethylation450 or MethylationEPIC BeadChip array. As the quality of sequenced reads may be heterogenous, a second quality filter based on mapping accuracy was employed: Only CpGs that were present on a base-called read with at least 95% mapping identity to the human reference genome were introduced into the computational model of the present invention. The mapping information additionally served for CNV classification according to the present invention, and the results are plotted.

### Example 7: Classifier Optimization and Evaluation-Based Copy Number Variation and Methylation in Cancer

Djiracktor et al. 2021 have nanopore sequenced brain tumors from 105 patients with shallow coverage. From the same tumors, standard of care neuropathological diagnoses according to the WHO 2016 classification system of central nervous tumors was obtained. The data was analysed by the authors with ad hoc trained Random Forest classification trees based on the sequenced CpGs, which were also present in the dataset from Capper et al. 2018. The methodology for the ad hoc training of Random Forest Classifiers as described in Kuschel et al. 2021 was used. As a result, a diagnosis concordant between the neuropathological diagnosis and the Random Forest classification was achieved in 93 of the 105 cases (89% concordance, for further details see the study by Djitacktor et a. 2021). The authors state in their study that with the chosen methodology results could be available from biopsy to computational result between 91 and 161 minutes with a median time of 97 minutes.

Raw nanopore sequencing data from this study was obtained from the authors from 101 of the published cases. The data was base-called, aligned and CpG methylation determined in Megalodon 2.3.2 (Software developed and provided by Oxford Nanopore Technologies). The Basecaller employed by Megalodon was Guppy 4.4.2. The artificial neural network model utilized by Megalodon and Guppy for basecalling was from Rerio “res_dna_r941_min_modbases_5mC_CpG_v001.cfg”. Alignment in Megalodon was performed MiniMap2. The reference genome utilized for alignment was GRCh38.p13.

A computational model in the form of a linear classifier with independent feature sampling trained on the dataset from Capper et al. 2018 and trained on Chromosomal p- and q-arms of all autosomes from the Capper et al. 2018 dataset was used to determine sample classes and diagnoses from the epigenetic information in the form of CpG methylation calls from the raw nanopore data obtained from Djitracktor and colleagues. Only CpGs were used for the computational model, which were present on nanopore reads with a mapping identity higher than 95% as determined by MiniMap2. Sample class probabilities were reported by the computational model for the five highest scoring sample class probabilities.

From all 101 obtained samples, 82 diagnosis and classification results for the highest scoring sample class were consistent with the standard of care neuropathological diagnosis reported in the study by Djitracktor and colleagues (Overall consistency: 81,19%).

For the further evaluation of these results, the inventors found that the dataset from Djitracktor et al. 2021 was heterogenous regarding the number of reads per sample (20,250 to 472,000 base-called) reads and the base calling quality was overall relatively low as assessed by quality metrics provided by the ONT software Guppy/Megalodon and were score with quality scores QC7 to QC11. In regular sequencing experiments, most reads are reaching QC15 quality scores with higher numbers indication higher quality. Notably, the tumor cellularity as stated by the authors was significantly lower than those values reported for the samples included in the Study of Capper et al. 2018.

It was found that the consistency of a diagnosis obtained from the computational model in the form of a linear classifier with independent feature sampling strongly correlated with the disease class percentage regularized to 300 simulated CpGs from the training dataset. It was empirically determined that a regularized percentage of above 60% for the highest disease class probability results in 59 samples classified consistently with the neuropathological diagnosis and only 4 samples were inconsistent with the neuropathological diagnosis (93.22% consistency). Conversely, regarding the 42 samples with a regularized class probability under 60% were in 27 cases consistent with the neuropathological diagnosis and 12 cases were inconsistent with the neuropathological diagnosis (64.29% consistency).

The following Tables summarizes the results of the method as described in the present invention: Table 1 lists all 101 cases, their integrated neuropathological diagnosis according to the WHO 2016 classification and the sequencing results as well as the diagnostic results obtained through the application of the present invention using genetic and epigenetic information from the data obtained from the study by Djitracktor et al. 2021. Genomic position specific genetic information utilized in the example are read counts on the arms of each Chromosomes p-arm and/or q-arm). Position specific epigenetic information utilized in the present example are CpG methylation information from CpGs present on the 450k Illumina Infinium Array. Table 2 demonstrates that the overall consistency of diagnosis with the conventional integrated neuropathological diagnosis obtained by the present invention increases by combining genomic position specific genetic and epigenetic information.

TABLE 1

| | | | | | no. | | | MZ Diagnosis |
|---|---|---|---|---|---|---|---|---|
| | | Tumor | | | CpGs | MZ | MZ CNV | Concordance with |
| | | cellularity | no. | no. of | used in | regularized | combined | WHO integrated |
| ID | WHO integrated diagnosis (WHO 2016) | ACE | reads | CpGs | classifier | percentage | Class | Diagnosis |
| NDMA_1 | Glioblastoma (WHO grade IV), IDH-wildtype | 4 | 111102 | 40206 | 7785 | 16.3% | GBM, MES | Concordant |
| NDMA_2 | Anaplastic oligodendroglioma (WHO grade III); IDH-mutant and 1p/19q-codeleted | 72 | 165271 | 78823 | 16677 | 59.5% | O IDH | Concordant |
| NDMA_3 | Anaplastic astrocytoma (WHO grade III), IDH-mutant | 10 | 20177 | 10477 | 1218 | 54.3% | O IDI | Discordant |
| NDMA_4 | Papillary craniopharyngioma | 100 | 192445 | 109975 | 19400 | 21.8% | CPH, PAP | Concordant |
| NDMA_5 | Rest/recurrence ganglioglioma (WHO grade I) | 25 | 256269 | 163860 | 24538 | 20.4% | LGG, PA/GG ST | Concordant |
| NDMA_6 | Glioblastoma (WHO grade IV), IDH-wildtype | 58 | 87727 | 58268 | 19054 | 45.3% | PXA | Discordant |
| NDMA_7 | Oligodendroglioma (WHO grade II); IDH-mutant and 1p/19q-codeleted | 4 | 81107 | 41577 | 5227 | 62.7% | IDF | Concordant |
| NDMA_8 | Glioblastoma (WHO grade IV), IDH-wildtype | 88 | 113004 | 34654 | 12050 | 84.5% | GBM, RTK I | Concordant |
| NDMA_9 | Paraganglioma (WHO grade I) | 15 | 67268 | 36708 | 7032 | 45.1% | SCHW, MEL | Discordant |
| NDMA_10 | Diffuse large B-cell lymphoma | 56 | 75950 | 32984 | 11292 | 100.0% | LYMPHO | Concordant |
| NDMA_11 | Oligodendroglioma (WHO grade II); IDH-mutant and 1p/19q-codeleted | 90 | 126613 | 22453 | 5864 | 86.8% | O IDH | Concordant |
| NDMA_12 | Anaplastic astrocytoma (WHO grade III), IDH-mutant | 32 | 304149 | 8540 | 2054 | 56.3% | A IDH | Concordant |
| NDMA_13 | Diffuse astrocytoma (WHO grade II), IDH-mutant | 5 | 171820 | 18144 | 2646 | 21.7% | LGG, DNT | Discordant |
| NDMA_14 | Anaplastic astrocytoma (WHO grade III), IDH-mutant | 38 | 153188 | 17758 | 3008 | 45.5% | A IDH | Concordant |
| NDMA_15 | Anaplastic astrocytoma (WHO grade III), IDH-mutant | 65 | 108163 | 25884 | 5296 | 67.9% | A IDH | Concordant |
| NDMA_16 | Oligodendroglioma (WHO grade II); IDH-mutant and 1p/19q-codeleted | 35 | 217347 | 25564 | 4475 | 19.3% | LGG, DNT | Discordant |
| NDMA_17 | Anaplastic astrocytoma (WHO grade III), IDH-mutant | 30 | 199858 | 13329 | 2834 | 44.6% | A IDH, HG | Concordant |
| NDMA_18 | Anaplastic medulloblastoma (WHO grade IV), SHH-activated, TP53-mutant | 48 | 27513 | 15917 | 894 | 97.4% | MB, SHH CHL AD | Concordant |
| NDMA_19 | Classic medulloblastoma (WHO grade IV), non-WNT/non SHH | 68 | 77358 | 51390 | 7079 | 100.0% | MB, G4 | Concordant |
| NDMA_20 | Medulloblastoma (WHO grade IV), SHH-activated, TP53-wildtype | 85 | 46805 | 23167 | 3719 | 98.7% | MB, SHH INF | Concordant |
| NDMA_21 | Classic medulloblastoma (WHO grade IV), non-WNT/non SHH, subclass 3 | 42 | 78034 | 24895 | 4132 | 100.0% | MB, G3 | Concordant |
| NDMA_22 | Classic medulloblastoma (WHO grade IV), WNT-activated | 85 | 113220 | 48610 | 7688 | 100.0% | MB, WNT | Concordant |
| NDMA_23 | Medulloblastoma (WHO grade IV), non-WNT/non SHH, subclass 3 | 3 | 93295 | 24136 | 3934 | 98.1% | MB, G3 | Concordant |
| NDMA_24 | Classic medulloblastoma (WHO grade IV), non-WNT/non SHH | 100 | 198443 | 43390 | 6686 | 94.2% | MB, G3 | Concordant |
| NDMA_25 | Classic medulloblastoma (WHO grade IV), non-WNT/non SHH, subclass 4 | 88 | 120597 | 3750 | 5619 | 100.0% | MB, G4 | Concordant |
| NDMA_26 | Malignant sarcomatous tumor with BCOR duplication | 100 | 90301 | 30039 | 367 | 100.0% | HGNET, BCOR | Concordant |
| NDMA_27 | High grade glioma, H3F3A-wildtype | 88 | 164843 | 31293 | 472 | 100.0% | GBM, G34 | Concordant |
| NDMA_28 | High grade glioma | 85 | 118487 | 19941 | 2978 | 45.9% | ATRT, SHH | Discordant |
| NDMA_29 | High grade glioma | 35 | 35284 | 7613 | 547 | 99.9% | MB, G3 | Discordant |
| NDMA_30 | Desmoplastic medulloblastoma (WHO grade IV), SHH-activated, TP53-wildtype | 94 | 64366 | 1747 | 3142 | 95.7% | MB, SHH INF | Concordant |
| NDMA_31 | Classic medulloblastoma (WHO grade IV), NOS | 36 | 87806 | 19133 | 2703 | 100.0% | MB, WNT | Concordant |
| NDMA_32 | Classic medulloblastoma (WHO grade IV), non-WNT/non SHH subclass group 3/4, subtype | 63 | 41931 | 5468 | 458 | 99.9% | MB, G3 | Concordant |
| NDMA_33 | Classic medulloblastoma (WHO grade IV), non-WNT/non SHH, subclass 4 | 91 | 136552 | 59735 | 3778 | 100.0% | MB, G4 | Concordant |
| NDMA_34 | Classic medulloblastoma (WHO grade IV), non-WNT/non SHH | 58 | 26222 | 13798 | 775 | 98.1% | MB, G4 | Concordant |
| NDMA_35 | Classic medulloblastoma (WHO grade IV), non-WNT/non SHH | 100 | 46882 | 24761 | 1325 | 97.8% | MB, G4 | Concordant |
| NDMA_36 | Classic medulloblastoma (WHO grade IV), non-WNT/non SHH, subclass3 | 42 | 50130 | 27063 | 1735 | 100.0% | MB, G3 | Concordant |
| NDMA_37 | Classic medulloblastoma (WHO grade IV), WNT-activated | 6 | 17926 | 7370 | 1202 | 100.0% | MB, WNT | Concordant |
| NDMA_38 | Metastatic medulloblastoma, non-WNT/non SHH, subclass 3 | 60 | 32044 | 7773 | 1248 | 99.3% | MB, G3 | Concordant |
| NDMA_39 | Anaplastic oligodendroglioma (WHO grade III); IDH-mutant and 1p/19q-codeleted | 71 | 257755 | 151118 | 30309 | 78.0% | IDH | Concordant |
| NDMA_40 | Glioblastoma (WHO grade IV), IDH-wildtype | 60 | 309245 | 186850 | 42909 | 80.7% | GBM, RTK I | Concordant |
| NDMA_41 | Glioblastoma (WHO grade IV), IDH-wildtype | 38 | 230600 | 16078 | 4043 | 19.4% | SCHW, MEL | Discordant |
| NDMA_43 | Oligodendroglioma (WHO grade II); IDH-mutant and 1p/19q-codeleted | 5 | 121189 | 31487 | 6260 | 79.3% | IDH | Concordant |
| NDMA_44 | Schwannoma | 67 | 129643 | 26334 | 5516 | 68.3% | SCHW | Concordant |
| NDMA_45 | Anaplastic oligodendroglioma (WHO grade III); IDH-mutant and 1p/19q-codeleted | 66 | 119295 | 22374 | 4532 | 73.2% | O IDH | Concordant |
| NDMA_46 | Glioblastoma (WHO grade IV), IDH-wildtype | 5 | 140993 | 91573 | 20552 | 93.5% | GBM, RTK I | Concordant |
| NDMA_47 | Ependymoma (WHO grade II) | 6 | 127449 | 14433 | 3548 | 97.9% | EPN, PF B | Concordant |
| NDMA_48 | Glioblastoma, IDH-wildtype, molecularly defined | 51 | 119924 | 1470 | 2967 | 22.3% | GBM, RTK | Concordant |
| NDMA_49 | Rest/recurrence GBM (WHO grade IV) | 66 | 208397 | 80187 | 33318 | 75.0% | GBM, RTK | Concordant |
| NDMA_50 | Glioblastoma (WHO grade IV), IDH-wildtype | 35 | 218272 | 117060 | 51527 | 17.0% | GBM, MES | Concordant |
| NDMA_52 | Diffuse midline glioma (WHO grade IV), H3 K27M-mutant | 51 | 247127 | 47008 | 16927 | 68.6% | GBM, MID | Discordant |
| NDMA_53 | LGG, best compatible with PA (WHO grade I) | 10 | 168465 | 67301 | 10961 | 14.6% | LGG, PA/GG ST | Concordant |
| NDMA_55 | Pilocytic astrocytoma (WHO grade I) | 30 | 129619 | 49391 | 11561 | 40.1% | LGG, PA PF | Concordant |
| NDMA_56 | Pilomyxoid astrocytoma | 49 | 136853 | 23468 | 5582 | 20.8% | LGG, PA MID | Concordant |
| NDMA_57 | Diffuse astrocytoma, IDH-wildtype (pediatric type) | 27 | 146733 | 82910 | 13717 | 21.4% | LGG, PA/GG ST | Discordant |
| NDMA_58 | Pilocytic astrocytoma (WHO grade I) | 67 | 94738 | 29437 | 5891 | 55.1% | LGG, PA PF | Concordant |
| NDMA_60 | Glioblastoma (WHO grade IV), IDH-wildtype | 36 | 463829 | 63141 | 319 | 64.2% | GBM, RTK II | Concordant |
| NDMA_62 | Pilomyxoid astrocytoma | 35 | 110997 | 27587 | 6348 | 98.4% | MB, G3 | Discordant |
| NDMA_63 | Glioblastoma (WHO grade IV), IDH-wildtype | 58 | 67039 | 19073 | 5490 | 89.9% | GBM, G34 | Concordant |
| NDMA_64 | Glioblastoma (WHO grade IV), IDH-wildtype | 44 | 100876 | 39048 | 7580 | 16.8% | GBM, MES | Concordant |
| NDMA_65 | Pilocytic astrocytoma (WHO grade I) | 66 | 151415 | 39876 | 7798 | 40.6% | LGG, PA PP | Concordant |
| NDMA_66 | Glioblastoma (WHO grade IV), IDH-mutant | 82 | 235107 | 159242 | 18549 | 80.5% | A IDH | Concordant |
| NDMA_67 | Diffuse midline glioma (WHO grade IV), H3 K27M-mutant | 5 | 116756 | 30214 | 13008 | 83.8% | DMG, K27 | Concordant |
| NDMA_68 | Diffuse astrocytoma, molecular GBM (WHO grade IV) | 23 | 145191 | 110856 | 28389 | 20.8% | GBM, MES | Concordant |
| NDMA_69 | Classic medulloblastoma (WHO grade IV), non-WNT/non SHH | 35 | 110997 | 27587 | 6348 | 98.4% | MB, G3 | Concordant |
| NDMA_70 | Ganglioglioma (WHO grade I) | 30 | 150860 | 58528 | 24657 | 31.3% | GG, PA/GG ST | Concordant |
| NDMA_71 | Oligodendroglioma (WHO grade II); IDH-mutant and 1p/19q-codeleted | 68 | 74853 | 22312 | 5515 | 74.3% | O IDH | Concordant |
| NDMA_72 | Low-grade neuroepithelial tumor, possibly pilocytic astrocytoma (WHO grade I) | 43 | 407392 | 317435 | 114554 | 30.8% | LGG, DNT | Concordant |
| NDMA_73 | Rest/recurrence anaplastic ependymoma (WHO grade III), C11ORF95-MAML2 fusion | 6 | 152889 | 3657 | 3132 | 34.0% | EPN, RELA | Concordant |
| NDMA_74 | Pilocytic astrocytoma (WHO grade I) | 5 | 181151 | 34702 | 7901 | 42.5% | LGG, PA PF | Concordant |
| NDMA_75 | Diffuse astrocytoma (WHO grade II), IDH-mutant | 43 | 74943 | 27470 | 6859 | 76.3% | A IDH | Concordant |
| NDMA_76 | Astrocytoma, IDH-wildtype | 65 | 245921 | 59439 | 18129 | 59.6% | DLGNT | Discordant |
| NDMA_78 | Classic medulloblastoma (WHO grade IV), SHH activated and TP53 wildtype | 36 | 57538 | 34713 | 15040 | 53.8% | MB, SHH CHL AD | Concordant |
| NDMA_79 | Glioblastoma (WHO grade IV), IDH-mutant | 100 | 150481 | 58052 | 9137 | 80.1% | A IDH, HG | Concordant |
| NDMA_80 | Glioblastoma (WHO grade IV), IDH-wildtype | 15 | 235630 | 63361 | 12672 | 23.2% | LGG, SEGA | Discordant |
| NDMA_81 | Oligodendroglioma (WHO grade II); IDH-mutant and 1p/19q-codeleted | 38 | 47609 | 22753 | 645 | 55.6% | IDF | Concordant |
| NDMA_82 | Anaplastic oligodendroglioma (WHO grade III); IDH-mutant and 1p/19q-codeleted | 66 | 417600 | 37263 | 7895 | 50.4% | O IDH | Concordant |
| NDMA_83 | Glioblastoma (WHO grade IV), IDH-wildtype | 13 | 197501 | 97278 | 11999 | 16.0% | LGG, PA PF | Discordant |
| NDMA_84 | Rest/recurrence GBM (WHO grade IV), IDH-wildtype with granular cell component | 6 | 97646 | 15567 | 1837 | 19.6% | CONTR, HEMI | Discordant |
| NDMA_85 | Meningioma (WHO grade I) | 35 | 119395 | 12267 | 1902 | 49.3% | MNG | Concordant |
| NDMA_86 | Oligodendroglioma (WHO grade II); IDH-mutant and 1p/19q-codeleted | 50 | 67555 | 62845 | 16456 | 29.0% | O IDH | Concordant |
| NDMA_87 | Glioblastoma (WHO grade IV), IDH-wildtype | 35 | 227044 | 33311 | 8148 | 46.0% | GBM, MES | Concordant |
| NDMA_88 | Rest/recurrence anaplastic meningioma (WHO grade III) | 15 | 48839 | 4426 | 1310 | 94.6% | SFT HMPC | Discordant |
| NDMA_89 | Medulloblastoma (WHO grade IV), non-WNT/non SHH | 44 | 79299 | 65314 | 17630 | 100.0% | MB, G4 | Concordant |
| NDMA_90 | Classic Medulloblastoma (WHO grade IV), WNT-activated | 65 | 125471 | 14650 | 5112 | 100.0% | MB, WNT | Concordant |
| NDMA_91 | Oligodendroglioma (WHO grade II); IDH-mutant and 1p/19q-codeleted | 80 | 46799 | 53767 | 20479 | 78.3% | O IDH | Concordant |
| NDMA_92 | Rest/recurrence epitheliod GBM, BRAFV600E mutant | 35 | 309520 | 287888 | 74740 | 25.5% | LGG, SEGA | Discordant |
| NDMA_93 | Atypical choroid plexus papilloma (WHO grade II) | 79 | 106115 | 148473 | 25540 | 65.2% | PLEX, PED B | Concordant |
| NDMA_94 | Classic medulloblastoma (WHO grade IV), non-WNT, non-SHH | 39 | 190373 | 169529 | 33950 | 100.0% | MB, G4 | Concordant |
| NDMA_95 | Diffuse midline glioma (WHO grade IV), H3 K27M-mutant | 61 | 134174 | 141937 | 47672 | 92.9% | DMG, K27 | Concordant |
| NDMA_96 | Rest/recurrence pilocytic astrocytoma(WHO grade I) | 55 | 285954 | 96025 | 13956 | 33.3% | LGG, RGNT | Discordant |
| NDMA_97 | Anaplastic astrocytoma (WHO grade III), IDH-mutant | 46 | 98930 | 97350 | 19941 | 81.5% | A IDH | Concordant |
| NDMA_98 | Atypical teratoid/rhabdoid tumor (WHO grad IV) | 43 | 157757 | 83053 | 16597 | 82.0% | ATRT, SHH | Concordant |
| NDMA_99 | Atypical teratoid/rhabdoid tumor (WHO grad IV) | 100 | 32644 | 33091 | 3119 | 80.6% | ATRT, SHH | Concordant |
| NDMA_100 | Classic medulloblastoma (WHO grade IV), WNT-activated | 96 | 58854 | 19417 | 6916 | 100.0% | MB, WNT | Concordant |
| NDMA_101 | Glioblastoma (WHO grade IV), IDH-wildtype | 54 | 72374 | 57531 | 23881 | 87.2% | GBM, RTK | Concordant |
| NDMA_102 | Oligodendroglioma (WHO grade II); IDH-mutant and 1p/19q-codeleted | 50 | 285779 | 177234 | 23633 | 81.1% | O IDH | Concordant |
| NDMA_103 | Glioblastoma (WHO grade IV), IDH-mutant | 51 | 63467 | 65908 | 11503 | 64.7% | A IDH | Concordant |
| NDMA_104 | Glioblastoma (WHO grade IV), IDH-wildtype | 48 | 115637 | 87371 | 24146 | 15.5% | GBM, MES | Concordant |
| NDMA_105 | Astroblastoma | 64 | 89755 | 55212 | 17753 | 59.6% | A IDH | Discordant |
| NDMA_106 | Anaplastic oligodendroglioma (WHO grade III); IDH-mutant and 1p/19q-codeleted | 73 | 27199 | 14150 | 809 | 90.1% | O IDH | Concordant |
| NDMA_107 | Schwannoma | 48 | 55616 | 22140 | 2258 | 95.4% | SCHW | Concordant |

NMDA: Data from Djitracktor et al. 2021.;
MZ: Method as described in the invention;
no = number

TABLE 2

| | MZ: CNV Combined with | | | MZ: Only | | |
|---|---|---|---|---|---|---|
| | Methylation | | | Methylation | | |
| | All | >=60% | <60% | All | >=60% | <60% |
| Total | 101 | 59 | 42 | 101 | 59 | 42 |
| Concordant | 82 | 55 | 27 | 80 | 55 | 25 |
| Discordant | 19 | 4 | 15 | 21 | 4 | 17 |
| Concordancy | 81.19% | 93.22% | 64.29% | 79.21% | 93.22% | 59.52% |

legend:
MZ—Method as described in the invention,
CNV—copy number variation

### Example 8: Application of the Present Invention to Selective Sequencing and Selective Resequencing Via Targeted Nucleases

Oxford Nanopore Technologies (ONT) has developed two approaches to enrich target sequences in DNA:

One method termed “selective sequencing” uses the ability of a nanopore system to eject undesired DNA molecules from the nanopore through a reversal of the voltage across the nanopore and to continue sequencing desired DNA molecules by not interrupting the sequencing process (Loose et al., 2016). This method has also been termed “read until” and several software implementations exist on the ONT MinION platform (Edwards et al., 2019, Payne et al., 2021; Stevanovski et al., 2022)

The decision in this approach, if a DNA sequence is “desirable” for a specific application or not, can be made based on specific DNA sequences, as has been demonstrated before in previous work by others. In one example, a catalog of “desirable” regions in the human genome has been defined a priori by a user. A DNA sample from a human individual is sequenced, and the reads are base called and aligned to the human genome. If the sequence belongs to a “desirable” genomic region, the sequencing of said sequence is continued. Suppose the sequence belongs to a not “desirable” region of the human genome. In that case, the voltage over the nanopore is reverted, and the DNA molecule with the “undesirable” DNA molecule is ejected. After reverting the voltage over the nanopore again, another DNA molecule can be sequenced by the nanopore. Again, it can be decided if a DNA molecule is “desirable” or not “desirable.” As a result, said “desirable” genomic regions can be enriched selectively over those considered not “desirable.” This process is repeated iteratively until a sequencing result has been obtained.

This well-established concept and existing nanopore workflow can be improved through the application of the present invention: In a suspected disease, DNA is obtained from a patient. Again, based on the clinically suspected diagnosis, a “desirable” set of genomic regions to be sequenced preferably is defined, and the nanopore sequencing process is initiated. As described, DNA molecules are sequenced and based on those short fragments obtained by sequencing tens to a few hundreds of base pairs from each molecule and alignment to the human genome, it is determined, if a DNA sequence belongs to the “desirable” or not “desirable” genomic regions. Even though the sequencing process becomes after the decision is made, if a sequence is “desirable” or not “desirable”, selective to a predefined subset of genomic positions, the initial step of indiscriminately sequencing all DNA molecules until enough sequence information is available to decide, if a DNA molecule is fully sequenced or ejected, conforms to the Bernoulli and/or Poisson sampling conditions as outlined in the present invention. The sequence and epigenetic information contained in the short fragments are recorded by the system and can be utilized to derive SNP and mutation calls, CNV profiles, as well as CpG methylation calls. (“Classification of tumours of the central nervous system using nanopore sequencing. Oxford Nanopore Technologies (as retrieved on Sep. 6, 2022 at https://nanoporetech.com/sites/default/files/s3/posters/lc2022/CNS % 20v1.1%20digital.pdf).

Applying this genetic and epigenetic information to the present invention to improve the diagnostic value of selective sequencing is straightforward: The epigenetic and/or genetic information contained in the short and long reads from a selective sequencing nanopore workflow can be used to determine sample classes and thus, medically relevant and clinically actionable diagnoses.

Notably, in the context of the present invention, such sample class, and diagnostic information can trigger the sequencing system to adjust the definition of the “desirable” and “undesirable” genomic regions to said sample classification problem. Adjusting the “desirable” and “undesirable” genomic regions can be used to further and more rapidly exclude differential diagnoses and refocus on relevant secondary genomic regions that may hold diagnostically relevant genomic information specific for a diagnostic subclass.

For example, a selective sequencing run is started on a brain tumor sample from a person. The MinION sequencing apparatus is set to selectively resequence all genomic regions known to contain diagnostic relevant information relating to brain tumors such as the MGMT-promotor and IDH1/2-, ATRX-genes. At the same time, CpG analysis of all genetic and epigenetic information, including the short sequencing reads from ejected DNA molecules with a computational model described herein, indicates the presence of a glioblastoma RTK-I subtype.

As the next step, the computational model can automatically trigger the re-definition of the “desirable” regions to include those with, for example, pharmacogenomic relevance for the treatment of an IDH1/2 negative glioblastoma and provide individualized to the patient genetic and epigenetic evidence on receptor tyrosine kinase genes (for instance EGFR-, IGFR-, PDGFR- and VEGFR-genes) to allow for targeted pharmacologic treatments tailored towards this specific patient. Similarly, a set of genomic regions containing genes with known immunogenic mutations in glioblastoma can be triggered to be selectively sequenced as directed by the information provided by the computational model as described in the present invention. Suppose this process finds an immunogenic mutation. In that case, this information can then, in turn, be used to develop an individualized, targeted immunization therapy with an mRNA vaccine targeting said patient's tumor.

In another example, DNA from a peripheral blood sample patient with the suspected diagnosis of septicemia is sequenced in an intensive care unit at the point of care because of the critical situation of said patient. The DNA from the peripheral blood contains both bacterial and human DNA. A selective sequencing approach can be used to enrich bacterial DNA over human DNA (Marquet et al., 2022) and to search for antibiotic resistance genes in the bacterial DNA to guide the administration of pharmacologic antibiotic therapy (Whittle et al., 2022). While the pores continuously eject human DNA fragments, epigenetic and genetic information from these human DNA fragments is continually sequenced, recorded, and collected based on the sampling processes as outlined in the present invention. According to the present invention, a computational model can continuously analyze the human epigenetic and genetic data obtained from DNA fragments rejected as “not desirable” and compute prognostic disease classes for said patient based on her epigenetic and/or epigenetic information. Such disease classes can encompass but are not limited to diagnostic information of the activation of specific immune cells, the activation of the blood clotting system, and prognostic score for the likely course of the disease. This diagnostic information derived from the computational model can automatically trigger the addition of distinct genomic regions in the human genome to the “desirable” sequences to be selectively sequenced to determine pharmacogenomic information from said patient relevant for treatments of, e.g., disseminated intravascular coagulation, which is a complication from septicemia in critically ill patients. Similarly, distinct epigenetic diagnostic class probabilities could indicate the presence of another viral infection and subsequently enable the administration of antiviral drugs by the treating physicians.

A second method for targeted resequencing has been developed and is built on the molecular identification of distinct DNA sequences through marking said DNA sequences with a programmable nuclease (Giesselmann et al., 2019; Gilpatrick et al., 2020) and selectively attaching nanopore sequencing adaptors to those DNA fragments which have been marked by a targeted nuclease.

Specifically, a plurality of DNA fragments is brought into contact with an RNA-guided nuclease such as a CRISPR/Cas9-nuclease. The RNA contained in the ribonucleotide complex has been programmed in such a manner that genomic regions are being attached to an Cas9-ribonucleotide complex which is inactivated in its nuclease activity or alternatively are cut by an active Cas9 ribonucleotide process. In the former case, DNA fragments bound to Cas9 molecules can be enriched from the plurality of DNA molecules. In the latter case, a phospho-group is exposed by the nuclease function and nanopore sequencing adaptors can be selectively attached to the exposed phosphor-group of the marked DNA fragments, while the other DNA unmarked DNA molecules have been dephosphorylated.

Therefore, relatively more DNA fragments are being sequenced in a nanopore sequencing process, which have been either attached to an inactive Cas9-ribonucleotide or have been cut by an Cas9-ribonucleotide and selectively ligated to a nanopore sequencing adaptor.

If such targeted DNA fragments containing prespecified genomic regions are selectively targeted in a nanopore sequencing process, still up to 100000 fold more sequencing reads are sequenced by a nanopore flow cell. Although the nuclease guided targeting mechanism may be specific and enrich the targeted DNA fragments up to 1000-fold, millions of unspecific reads are sequenced in parallel (Giesselmann et al., 2019). See for more information Giesselmann et al. 2019, specifically FIG. 2d. This is due to the fact that in the case of the most efficient method for selective resequencing with Cas9-nucleases, many DNA fragments from other regions contain phospho-groups at their ends, resulting in unspecific binding of nanopore sequencing adaptors to these fragments covering all other genomic regions, which have not been targeted by Cas9-ribonucleotides.

In a diagnostic application, such epigenetic and/or genetic information derived from both the targeted DNA fragments as well as the other, not targeted genome-wide DNA fragments can be utilized by a computational model as described here within: CpG methylation, CNV information can be determined, and sample class probabilities resulting in a medical diagnosis can be computed from genetic and/or epigenetic information contained in both targeted and not targeted sequences.

For example, a targeted sequencing panel with Cas9-ribonucleotides targeting several hundred distinct genomic regions with relevant mutations and/or epigenetic diagnostic or therapeutic markers is applied to a brain tumor sample. Said targeted genomic regions may contain known single base mutations in brain cancers, such as the TERT promotor, the IDH1/2 genes, or the entirety of the ATRX gene (Gilpatrick et al., 2020). Such a Cas9-ribonucleotide panel may also target fusion genes known for malignancies (Stangl et al., 2020).

Based on the genetic and/or epigenetic information contained in both the targeted and not targeted genomic regions, the computational model as described in the present invention can determine the diagnosis of glioblastoma with an NTRK-fusion gene intraoperatively but can also determine in the same intraoperative nanopore sequencing process through either Cas9-targeted DNA fragment enrichment or selective resequencing as described before the presence of an NTRK-fusion gene in said glioblastoma. Such fusion genes are only present in about 0.3-3% of all adult glioblastomas. Several NTRK-fusion gene inhibitors have recently become available. Current evidence points toward a clinically meaningful response of NTRK-fusion gene associated glioblastomas to NTRK-fusion gene inhibitors, which may cure the patient from the otherwise deadly disorder. Therefore, instead of extensive brain tumor resection, a primarily chemotherapeutic approach to treating the said patient can be initiated.

Based on the intraoperative results from a computational model described herein, neurological damage in the said patient can be avoided.

### Example 9: Prototype Development for Binary Disease Classification and Diagnosis-Application to Rheumatoid Arthritis

Previously, it has been demonstrated that CpG methylation patterns can be used to distinguish patients with rheumatoid arthritis (RA) from unaffected individuals (Liu et al., 2013). While the authors Liu, Feinberg et al. utilized the dataset for developing a mechanistic understanding of epigenetic modification of the disease process, the same dataset can be used to train and validate the computational model of the present invention for predicting the presence or absence of RA in a patient.

In general, training a Naïve Bayes Classifier given the class centroids and testing its classification accuracy on sparse samples derived from high coverage data is relatively easy to implement since there are not many parameters to choose. This situation is encountered in the case of the brain tumor cancer dataset published by Capper et al. 2018.

If the training data is larger or if the geometric structure of the data is not dominated by the disease other linear classification methods as outlined in the present invention can lead to satisfactory classification accuracy and sensitivity.

For linear classification multiple options of suitable algorithms are available. Glmnet is a popular solution for generalized linear models including binomial and multinomial logistic regression. Its alpha parameter allows to interpolate between a “lasso” and a “ridge” regularization penalty. At each alpha parameter a regularization path is produced and the optimal position in this path is chosen by cross-validation.

The optimal use of Glmnet is derived on the high coverage data and that it is not clear that a good classifier on high-coverage data works well sampled positions, i.e. the Lasso penalty forces the majority of the weights to 0, so that the best performing classifier might only use 10-20 features even if there are hundred thousands available. Using such a low number of features obviously increases the sampling variability between different low coverage samples from the same patient.

It is therefore desirable to be able to evaluate the performance of the classifiers for multiple values of the regularization parameter on low coverage samples from existing patient samples in the training data via cross-validation. Since each patient sample needs to be sampled multiple times this can be expensive to implement.

To facilitate the development of linear classifiers with independent feature sampling, a method based on normal approximation has been developed, which allows to calculate the expectations and standard deviations for each sample under a Poisson or Bernoulli sampling regime if the number of non-zero parameters is not too small. If the weight matrix of the linear model contains only a few 100 non-zero values than sampling can be very efficiently performed.

Envision a very simple linear classifier where Y=t(W)X+c, where W is a weight matrix of dimension 2×n X is a Data matrix containing n features across m patients, c is a 2×1 vector containing the constant intercept values. Y is the 2×m vector containing the two Class scores for each patient in its columns. Classification is done by deciding for each Patient depending on the sign of y_1−y_2. The weight matrix is fixed and could be derived by optimizing an objective function, or by transforming a naive Bayes model in linear form.

A normal approximation is feasible because the inclusion probabilities are independently Bernoulli or Poisson sampled, as the individual observation of features (a single observation of CpG methylation at a distinct genomic position) follows a Bernoulli distribution, and the weights are fixed; (see also the appended example code).

While the same approximation does not exist for more than two classes, Binary classification can be used to build a multi-class classifier (Hsu and Lin, 2002) in a one-versus-one or one-versus all fashion. Multiclass Naïve Bayes and some multi class linear classifiers have a “class-order-invariance” property which effectively allows them to be disassembled and reassembled into pairwise classifiers (Sulzmann et al., 2007).

### Application Too Rheumatoid Arthritis

The publicly available data submitted from (Liu et al., 2013) to NCBI Geo (GSE42861) was used. The RA dataset does not show strong clustering by disease status, the strongest association visible in the UMAP plots is gender (FIGS. 13 and 14).

First the data was subset to 100.000 out of 485512 positions and the patient population was restricted to patients who have never smoked leaving 92 RA cases and 101 normal controls.

The set was further split randomly in a training a test data set, leaving 135 samples for training and 58 for evaluation (70/30 Training Test split). Using the “binomial” option, 3 different values of the parameter alpha: 1, 0.5 and 0 were evaluated. On the training set the Misclassification error was estimated at 1SE as 0.1778, 0.2296 and 0.1926. Rank order: alpha=1 best, alpha=0 second and alpha 0.5 last. (Model summaries appended)

As 100.000 is still accessible to sampling, 100 sparse sampled versions were generated by generating 10 different random binary CpG methylation event vectors per patient sample and then performing 10 random subsamples of CpG positions using a Bernoulli sampling with an inclusion rate of 50%.

The average rate of correctly classified samples across 100 simulations on the training data is: 0.0.532, 0.56, 0.868 (alpha=0, 0.5. 1) and 0.55, 0.53, 0.7

The normal approximations at a rate of 0.5 were calculated and these are at alpha 1 (0.53 Training/0.52 Test), alpha 0.5 (0.56 Training/0.54 Test) and at alpha=0 (0.875 Training/0.69 Test), which demonstrates excellent concordance between simulated and calculated values based on the normal approximation.

The availability of a fast approximation allows to quickly estimate the misclassification rate for different model parameters. The result of this estimation is shown in FIG. 15.

Only a subset of less than 25% of the available data was used and in depth parameter optimization was not performed. Nonetheless, this demonstrated that the approach is not limited to the Naïve Bayes setting as significant correct classification rates were achieved using a generalized linear modeling method (regularized logistic regression). The selected model is a linear classifier with independent feature sampling.

### Glmnet Model Summary

resRAGLMcvTTrain1
Call: cv.glmnet(x=t(BetaRANever100k[, selTrain][,]), y=annotation$disease[selNever][selTrain], type.measure=“class”, family=“binomial”, alpha=1, intercept=TRUE)
Measure: Misclassification Error

| | Lambda | Index | Measure | SE | Nonzero |
|---|---|---|---|---|---|
| min | 0.00734 | 83 | 0.1482 | 0.03361 | 76 |
| 1se | 0.05424 | 40 | 0.1778 | 0.03311 | 46 |

resRAGLMcvTTrain.5
Call: cv.glmnet (x=t (BetaRANever100k[, selTrain][,]), y=annotation$disease[selNever][selTrain], type.measure=“class”, family=“binomial”, alpha=0.5, intercept=TRUE)
Measure: Misclassification Error

| | Lambda | Index | Measure | SE | Nonzero |
|---|---|---|---|---|---|
| min | 0.1574 | 32 | 0.2000 | 0.03391 | 72 |
| 1se | 0.5035 | 7 | 0.2296 | 0.03789 | 23 |

resRAGLMcvTTrain0
Call: cv.glmnet (x=t (BetaRANever100k[, selTrain][,]), y=annotation$disease[selNever][selTrain], type.measure=“class”, family=“binomial”, alpha=0, intercept=TRUE)
Measure: Misclassification Error

| | Lambda | Index | Measure | SE | Nonzero |
|---|---|---|---|---|---|
| min | 6.09 | 87 | 0.1630 | 0.03631 | 1e+05 |
| 1se | 119.60 | 23 | 0.1926 | 0.04272 | 1e+05 |

#-------------------------------
#
#Code
# Calculates row-wise expectations and variances of the feature observations at all all positions
# before sampling
# p_s is a vector of probabilities derived from a high-coverage patient sample. i.e. a CpG methylation
beta-value
# W1,W2 are the column vectors of the weight matrix
ExpVarDiffWMatrix <- function(p_s,W1,W2X{
E<−p_s*(W1−W2)
V<−p_s*(1−p_s)*(W1−W2)\hat{ }2
list(expectation=E,Variance=V)
}
#
#Using a formular for the multiplication of variances
#[Goodman1960]
# Var(X)= E(X)\hat{ }2 Var(Y)+E(Y)?2Var(X)+Var(X)Var(Y)
#X= sampling Inclusionvariable (bernouli/poisson) with rate := p_include)
# E(X)\hat{ }2 =rate\hat{ }2 ;Var(X)=(1-rate)(rate)
# E(Y)=E; Var(Y)=V
#Takes the Expectation and Variance from previous step and calculates the combine Expectation,
Variance and Standard deviations
ExpVarDiffSampling<−function(E,V,rate =.1){
if(lenght(rate==1) else EX<−rep(rate,length(E))
else EX<−rate
VX<−(1−EX)*EX
VarS<−sum(EX?2*V+E?2*VX+V*VX)
ES<−sum(E*rate)
c(ES,VarS,sqrt(VarS))
}

### Example 10: Intraoperative Diagnosis of Brain Tumors

Routine diagnostics of brain tumors typically require days or even weeks to complete. Thus, the potential of the present invention was evaluated, and an optimized molecular workflow for the rapid classification of brain tumors within the time frame of a standard neurological surgery next to the operating theatre was implemented. The samples described below represent a collection of samples obtained from the department of neurosurgery at the University Hospital Schleswig-Holstein (UKSH), and experiments were termed “clinical demonstrators.”

All samples were subjected to the same molecular workflow described in Example 1, and sequencing was performed on a MinION device by using a single flow cell (R9.4.1) for each experiment. The optimized DNA extraction protocol (tissue weigh, extraction, assessment of DNA quality and quantity) allowed the isolation of DNA with sufficient quality determined by Nanodrop (A260/280 ratio >1.8 and A260/230>2.0) and sufficient quantity (>50 ng/μl) within 22-23 min followed by a rapid sequencing library preparation within 16-17 min. The final sequencing step, including the time for temperature calibration, MUX scan, and sequencing of the flow cell, varied among all the samples based on tumor entity.

For analysis and methylation calling Megalodon 2.3.2 was used. Megalodon combines Guppy 4.4.2 for basecalling and minimap2 for mapping the base called sequences to a human reference genome (GRCh38.p13). Methylation data was read from the database file in real time and analyzed directly with the computational model. As a DNA fragment is sequenced in a nanopore, the resulting ionic current measurements are transformed and base called with deep neural networks. After aligning these sequences to a reference genome, more complex deep neural networks are utilized for methylation calling all CpGs present in a DNA sequence from the raw ionic current data in the Megalodon software. When new information on CpG methylation from a DNA molecule sequenced only milliseconds ago is available, said epigenetic information is input into the computational model.

Each new classification result was displayed with the number of reads analysed and the number of CpGs taken into account for the classification. Three brain tumors (IEG112, 114, and 123) out of four samples could be classified within 6-7 min of sequencing time with high certainty (>72%), resulting in an overall protocol time of 44-46 min from DNA extraction to tumor class prediction (Table 3). Accurate prediction of the underlying tumor class was possible with a relatively low number of CpGs ranging from 1869-3108 (Table 3). Computational model results for sample IEG108 were also concordant with clinical diagnosis after 5 min of sequencing (FIG. 16). Still, they lacked enough CpG information for a reliable, standardized classification, indicated by a low certainty score (<41%). However, prolonging sequencing time to 10 min allowed correct classification with a certainty of >70% within a total protocol time of 50 min. Most importantly, the predicted diagnosis for all four samples matched the clinical diagnosis throughout the entire time of the classification procedure (Table 4).

FIG. 16 displays snapshots from a continuous video recording of the sequencing of IEG 108 with the associated time stamps and steps in the sequencing process displayed in the photographs.

TABLE 3

| | | Minutes | no, | mapped | no, of | no, CpGs | | standardized class |
|---|---|---|---|---|---|---|---|---|
| ID | Clinical Diagnosis | sequenced | reads | reads | CpGs | used in classifier | Class | probability |
| IEG108 | Glioblastoma | 5 | 5583 | 5120 | 2167 | 2164 | GBM, RTK II | 40.79% |
| IEG112 | Meningioma | 5 | 4731 | 4346 | 1876 | 1869 | MNG | 99.45% |
| IEG114 | Oligodendroglioma | 5 | 6865 | 6273 | 3341 | 3108 | O IDH | 72.43% |
| IEG123 | Medulloblastoma | 5 | 4066 | 3849 | 1630 | 1618 | MB, G4 | 99.79% |
| IEG108 | Glioblastoma | 10 | 14745 | 13608 | 6618 | 6608 | GBM, RTK II | 70.06% |
| IEG112 | Meningioma | 10 | 12326 | 11429 | 6343 | 6274 | MNG | 99.50% |
| IEG114 | Oligodendroglioma | 10 | 15564 | 14239 | 8488 | 8140 | O IDH | 68.37% |
| IEG123 | Medulloblastoma | 10 | 10803 | 10241 | 5063 | 5047 | MB, G4 | 99.57% |
| IEG108 | Glioblastoma | 15 | 24237 | 22464 | 10968 | 10947 | GBM, RTK II | 79.10% |
| IEG112 | Meningioma | 15 | 19752 | 18417 | 11620 | 11534 | MNG | 99.88% |
| IEG114 | Oligodendroglioma | 15 | 24254 | 22175 | 13463 | 13096 | O IDH | 70.43% |
| IEG123 | Medulloblastoma | 15 | 18076 | 17134 | 9321 | 9293 | MB, G4 | 99.88% |

TABLE 4

| | | | | Time for | Time for | | |
|---|---|---|---|---|---|---|---|
| | | | | DNA | library | Sequencing | Total time to |
| Sample | Flow cell | Clinical | MethLYZR | extraction | preparation | time | classification |
| ID | ID | diagnosis | prediction/Class | [min] | [min] | [min] | [min] |
| IEG108 | FAQ67156 | Glioblastoma | Glioblastoma, GBM, RTK II | 23 | 17 | 10 | 50 |
| IEG112 | FAQ67187 | Meningioma | Meningioma, MNG | 22 | 17 | 7 | 46 |
| IEG114 | FAQ67345 | Oligodendroglioma | Oligodendroglioma, O IDH | 22 | 17 | 6 | 45 |
| IEG123 | FAR83286 | Medulloblastoma | Medulloblastoma, MB, G4 | 22 | 16 | 6 | 44 |

### FURTHER REFERENCES

1. Aref-Eshghi E, Kerkhof J, et al. Evaluation of DNA Methylation Episignatures for Diagnosis and Phenotype Correlations in 42 Mendelian Neurodevelopmental Disorders. Am J Hum Genet. 2020 Mar. 5; 106 (3): 356-370.
2. Monk D, Morales J et al. Recommendations for a nomenclature system for reporting methylation aberrations in imprinted domains. Epigenetics. 2018; 13 (2): 117-121.
3. Zemmour H, Planer D et al. Non-invasive detection of human cardiomyocyte death using methylation patterns of circulating DNA. Nat Commun. 2018 Apr. 24; 9 (1): 1443.
4. Cuadrat R R C, Kratzer A et al. Cardiovascular disease biomarkers derived from circulating cell-free DNA methylation. Medrxiv. 2021 Nov. 17; 21265870; https://doi.org/10.1101/2021.11.05.21265870
5. Liu Y, Arvee M J et al. Epigenome-wide association data implicate DNA methylation as an intermediary of genetic risk in rheumatoid arthritis. Nat Biotechnol. 2013 Jan. 20; 31 (2): 142-147.
6. Hollon T C, Pandian B et al. Near real-time intraoperative brain tumor diagnosis using stimulated Raman histology and deep neural networks. Nat Med. 2020 January; 26 (1): 52-58
7. Djirackor L, Halldorsson S et al. Intraoperative DNA methylation classification of brain tumors impacts neurosurgical strategy. Neuro-Oncology advances. 2021 Oct. 10; 3 (1): vdab149.
8. Capper D, Jones D T W et al. DNA methylation-based classification of central nervous system tumours. Nature. 2018 Mar. 22; 555 (7697): 469-474.
9. Suárez-Orozco C, Motti-Stefanidi F et al. An integrative risk and resilience model for understanding the adaptation of immigrant-origin children and youth. Am Psychol. 2018 September; 73 (6): 781-796.
10. Koelsche C, Schrimpf D et al. Sarcoma classification by DNA methylation profiling. Nat Commun. 2021 Jan. 21; 12 (1): 498.
11. Simpson J T, Workman R E et al. Detecting DNA cytosine methylation using nanopore sequencing. Nat Methods. 2017 April; 14 (4): 407-410. doi: 10.1038/nmeth.4184.
12. Katsman e, Orlanski S et al. Detecting cell-of-origin and cancer-specific methylation features of cell-free DNA from Nanopore sequencing. Genome Biol. 2022 Jul. 15; 23 (1): 158.
13. Li H, Feng X et al. The design and construction of reference pangenome graphs with minigraph. Genome Biol. 2020 Oct. 16; 21 (1): 265.
14. Kuschel L P, Hench J et al. Robust methylation-based classification of brain tumors using nanopore sequencing. medRxiv. 2021 Jun. 3; 21252627; doi: https://doi.org/10.1101/2021.03.03.21252627
15. Green M and Sambrook J. Molecular Cloning: A Laboratory Manual. 4th Edition, Vol. II, Cold Spring Harbor Laboratory Press, New York. 2012.
16. Lister R, Pelizzola M et al. Human DNA methylomes at base resolution show widespread epigenomic differences. Nature. 2009 Nov. 19; 462 (7271): 315-22.
17. Louis D N, Perry A et al. The 2021 WHO Classification of Tumors of the Central Nervous System: a summary. Neuro Oncol. 2021 Aug. 2; 23 (8): 1231-1251.
18. Ricklefs F L, Drexler R et al. DNA methylation subclass receptor tyrosine kinase II (RTK II) is predictive for seizure development in glioblastoma patients. Neuro-Oncology. 2022 May 3; noac108.
19. Drexler R, Schüller U et al. DNA methylation subclasses predict the benefit from gross total tumor resection in IDH-wildtype glioblastoma patients. Neuro-Oncology. 2022 Jul. 22; noac177.
20. Magi A, Bolognini D, Bartalucci N, Mingrino A, Semeraro R, Giovannini L, et al. Nano-GLADIATOR: real-time detection of copy number alterations from nanopore sequencing data. Bioinformatics 2019; 35:4213-21. https://doi.org/10.1093/bioinformatics/btz241.
21. Xie C and Tammi M. CNV-seq, a new method to detect copy number variation using highthroughput sequencing. BMC Bioinformatics. 2009 Mar. 6; 10 (80): 1471-2015.
22. Bie F, Wang Z, et al. Noninvasive cancer detection by extracting and integrating multimodal data from whole-methylome sequencing of plasma cell-free DNA. bioRxiv. 2022 Jul. 4; 498641; doi: https://doi.org/10.1101/2022.07.04.498641
23. Wick R R, Judd L M et al. Performance of neural network basecalling tools for Oxford Nanopore sequencing. Genome Biol. 2019 Jun. 24; 20 (1): 129.
24. Orozco et al. Epigenetic profiling for the molecular classification of metastatic brain tumors. Nature Comms. 2018 9:4627.
25. Edwards H S, Krishnakumar R et al. Real-Time Selective Sequencing with RUBRIC: Read Until with Basecall and Reference-Informed Criteria. Sci Rep-Uk, 2019 Aug. 7; 9:11475. https://doi.org/10.1038/s41598-019-47857-3
26. Gisselmann O, Brandl B et al. Analysis of short tandem repeat expansions and their methylation state with nanopore sequencing. Nature Biotechnology. 2019 Nov. 18; 37:1478-1481. https://doi.org/10.1038/s41587-019-0293-x.
27. Gilpatrick T, Lee I et al. Targeted nanopore sequencing with Cas9-guided adapter ligation. Nature Biotechnology. 2020 Feb. 10; 38:433-438. https://doi.org/10.1038/s41587-020-0407-5.
28. Loose M, Malla et al. Real-time sequencing using nanopore technology. Nat Methods. 2016 Jul. 25; 13:751-754. https://doi.org/10.1038/nmeth.3930.
29. Marquet M, Zollkau J et al. Evaluation of microbiome enrichment and host DNA depletion in human vaginal samples using Oxford Nanopore's adaptive sequencing. Sci Rep-Uk. 2022 Mar. 7; 12:4000. https://doi.org/10.1038/s41598-022-08003-8.
30. Payne A, Holmes N et al. Readfish enables targeted nanopore sequencing of gigabase-sized genomes. Nat Biotechnol. 2021 Nov. 30; 39:442-450. https://doi.org/10.1038/s41587-020-00746-x
31. Stangl C, Blank S et al. Partner independent fusion gene detection by multiplexed CRISPR-Cas9 enrichment and long read nanopore sequencing. Nat Commun. 2020 Jun. 5; 11:2861. https://doi.org/10.1038/s41467-020-16641-7
32. Stevanovski I, Chintalaphani S R et al. Comprehensive genetic diagnosis of tandem repeat expansion disorders with programmable targeted nanopore sequencing. Science Advances. 2022 Mar. 4; 8 (9). https://doi.org/10.1126/sciadv.abm5386
33. Whittle E, Yonkus J A et al. Optimizing Nanopore Sequencing for Rapid Detection of Microbial Species and Antimicrobial Resistance in Patients at Risk of Surgical Site Infections. Msphere. 2022 Feb. 16; 7: e00964-21. https://doi.org/10.1128/msphere.00964-21.
34. Friedman J, Hastie T et al. Regularization Paths for Generalized Linear Models via Coordinate Descent. 2010 Feb. 2; 33 (1): 1-22. 10.18637/jss.v033.i01, https://www.jstatsoft.org/v33/i01/.
35. Hsu C W and Lin C J. A comparison of methods for multi-class support vector machines. IEEE Transaction on Neural Networks. 2002; 415-425.
36. Sulzmann J n, Fürnkranz J et al. On pairwise Naive Bayer Classifiers. Machine Learning: ECML 2007 (eds. Kok J N et al.); 4701:371-381 (Springer Berlin Heidelberg, 2007).
37. Goodman, L A. On the exact variance of products. Journal of the American Statistical Association. 1960 December; 55 (292): 708-713. doi: 10.2307/2281592. JSTOR 2281592.
38. Capper D, Jones D T W et al. DNA methylation-based classification of central nervous system tumours. Nature. 2018 Mar. 14; 555:469-474. https://doi.org/10.1038/nature26000.
39. James G, Witten D, Hastie T and Tibshirani R. An introduction to statistical learning: with applications in R. Springer US. 2011: second edition.
40. Konigsberg I R, Barnes B et al. Host methylation predicts SARS-COV-2 infection and clinical outcome. Commun Medicine. 2021; 1 (1): 42.

1. A method for the diagnosis and/or classification of a disease in a subject based on the genetic and/or epigenetic information of a sample obtained from the subject, the method comprising the steps of:
a) providing data from said sample, wherein said data comprises genetic and/or epigenetic information of a random subset of genomic positions;
b) assigning said sample to a sample class based on genetic and/or epigenetic information of said random subset of genomic positions by employing a computational model, which discriminates a plurality of sample classes based on genetic and/or epigenetic information of a set of genomic positions comprising said random subset, wherein the computational model has been trained with pre-determined genetic and/or epigenetic information obtained from a plurality of pre-classified samples of known diseases and wherein said computational model processes the genetic and/or epigenetic information of a genomic position of said random subset independently of the genetic and/or epigenetic information of another genomic position of said random subset, wherein said computational model is preferably in the form of a linear classifier with independent feature sampling.

2. The method of claim 1, wherein the computational model in the form of a linear classifier with independent feature sampling is: a) a Naïve Bayes classifier model, or b) a logistic regression model or c) a linear support vector machine model.

3. The method of claim 1 or 2, wherein the random subset of genomic positions consists of at least 100, preferably at least 200, more preferably at least 500, more preferably at least 1000 and most preferably at least 2000 genomic positions.

4. The method of any one of claims 1 to 3, wherein the genetic and/or epigenetic information of the random subset of genomic positions of said sample is binary or alternatively non-binary.

5. The method of any one of claims 1 to 4, wherein the genetic and/or epigenetic information of the set of genomic positions is binary or alternatively non-binary.

6. The method of any one of claims 1 to 5, wherein the computational model is trained by optimization of the classification accuracy using a statistical model which relates the probability of observing binary or non-binary genetic and/or epigenetic information in a random subset of genomic positions to a set of pre-determined genetic and/or epigenetic information in the form of event rates obtained from a plurality of pre-classified samples of known diseases.

7. The method of any one of claims 1 to 6, wherein the computational model employs Bernoulli sampling and/or sparse Poisson sampling and optionally utilizes pre-determined, sample-class specific, genomic position-specific weights for the subsequent assignment of a sample to a sample class in step b).

8. The method of any one of claims 1 to 7, wherein in step b) the genetic and/or epigenetic information of a genomic position is obtained and processed only once by said computational model and Bernoulli sampling is employed or alternatively several observations of the genetic and/or epigenetic information of a genomic position are obtained and processed by said computational model and Poisson sampling is employed.

9. The method of any one of claims 1 to 8, wherein the number of genomic positions in said random subset of genomic positions of step a) increases continuously, thereby providing the genetic and/or epigenetic information in the form of a data stream and wherein the computational model in step b) processes the genetic and/or epigenetic information in the form of a data stream and updates the result of step b) at the same rate or close to the same rate at which more genetic and/or epigenetic information become available through said data stream and processes all genetic and/or epigenetic information available up to the timepoint of the update of step b).

10. The method of any one of claims 1 to 9, wherein the genetic and/or epigenetic information comprises information about:
a) DNA methylation,
b) single nucleotide polymorphisms,
c) histone modifications,
d) structural variations such as deletions, insertions, inversions, tandem repeat variations, substitutions, disruptions, or
e) copy number variations, chromosomal losses or super numerous chromosomes;
with respect to a reference genome.

11. The method of claim 10, wherein the genetic and/or epigenetic information comprises information on the methylation status of CpG dinucleotides.

12. The method of any one of claims 1 to 11, wherein the data from said sample in step a) is obtained by:
i) isolating genomic DNA from said sample,
ii) preparing a DNA library by fragmentation of said isolated genomic DNA,
iii) sequencing of the DNA fragments obtained in step ii), thereby determining the sequence of nucleotides of said DNA fragments,
iv) comparing the sequence of nucleotides for each individual DNA fragment with the sequence of nucleotides of a reference genome, and
v) determining the genetic and/or epigenetic information of a genomic position from each DNA fragment in comparison to said reference genome, thereby providing data comprising genetic and/or epigenetic information of a random subset of genomic positions.

13. The method of claim 12, wherein the DNA library in step ii) is obtained by fragmentation of said genomic DNA of step i) by a transposase, the addition of tagging adapters the DNA fragments cleaved by said transposase and the subsequent attachment of sequencing adapters to said added tagging adapters, with said sequencing adapters carrying a motor enzyme with a helicase functionality suitable for the initiation of nanopore sequencing of said transposase tagged genomic DNA.

14. The method of claim 12 or 13, wherein nanopore sequencing is used in step iii).

15. The method of any one of claims 1 to 14, wherein the subject is a human subject.

16. The method of claim 15, wherein the sample has been obtained intraoperatively.
